(19) United States
(12) Patent Application Publication
(10) Pub. No.: US 2022/0319638 A1
(43) Pub. Date: Oct. 6, 2022

(54) PREDICTING RESPONSE TO TREATMENTS IN PATIENTS WITH CLEAR CELL RENAL CELL CARCINOMA

(71) Applicants: BostonGene Corporation, Waltham, MA (US); Washington University, St. Louis, MO (US)

(72) Inventors: James Hsieh, St. Louis, MO (US); Alexander Bagaev, Moscow (RU); Natalia Miheecheva, Moscow (RU); Ekaterina Postovalova, Moscow (RU); Danil Stupichev, Yekaterinburg (RU); Kristina Perevoshchikova, Moscow (RU)

(21) Appl. No.: 17/691,041

(22) Filed: Mar. 9, 2022

Related U.S. Application Data
(60) Provisional application No. 63/158,825, filed on Mar. 9, 2021.

(51) Int. Cl. G16B 25/10; G16B 20/20; G16B 40/20; G16H 50/30

(52) U.S. Cl. CPC G16B 25/10; G16B 20/20; G16B 40/20; G16H 50/30

(57) ABSTRACT

Aspects of the disclosure relate to methods, systems, computer-readable storage media, and graphical user interfaces (GUIs) that are useful for characterizing subjects having certain cancers, for example renal cell carcinomas such as clear cell renal carcinoma (ccRCC). The disclosure is based, in part, on methods for determining the renal cancer (RC) tumor microenvironment (TME) type (RC TME type) of a renal cancer subject and the subject's prognosis and/or likelihood of responding to certain therapies (e.g., immunotherapy or tyrosine kinase inhibitors) based upon the renal cancer type determination.

### CROSS REFERENCE TO RELATED APPLICATIONS

This application claims the benefit under 35 U.S.C. § 119(e) of U.S. Provisional Application No. 63/158,825, filed Mar. 9, 2021, titled “PREDICTING RESPONSE TO TREATMENTS IN PATIENTS WITH CLEAR CELL RENAL CELL CARCINOMA”, the entire contents of which are incorporated by reference herein.

### BACKGROUND

Correctly characterizing the type or types of cancer a patient or subject has and, potentially, selecting one or more effective therapies for the patient can be crucial for the survival and overall wellbeing of that patient. Advances in characterizing cancers, predicting prognoses, identifying effective therapies, and otherwise aiding in personalized care of patients with cancer are needed.

### SUMMARY

Aspects of the disclosure relate to techniques for characterizing subjects having certain renal (kidney) cancers, such as clear cell renal carcinoma (ccRCC). The disclosure is based, in part, on methods for identifying the tumor microenvironment (TME) of a subject having renal cancer (e.g., ccRCC) by using gene expression data obtained from the subject to produce a renal cancer (RC) tumor microenvironment (TME) signature (referred to as an RC TME signature) that, when processed by methods disclosed herein, allows for assignment of an RC TME type to the subject. In some embodiments, the RC TME type of a subject is indicative of one or more characteristics of the subject (or the subject's cancer), for example the likelihood a subject will have a good prognosis or respond to a therapeutic agent such as an immunotherapy (also referred to as an IO agent) or a tyrosine kinase inhibitor (TKI). Aspects of the disclosure also relate to machine learning techniques for determining whether (e.g., the likelihood that) a subject will have a good prognosis or respond to an IO agent or TKI.

Accordingly in some aspects, the disclosure provides a method for determining a renal cancer (RC) tumor microenvironment (TME) type for a subject having, suspected of having, or at risk of having renal cancer, the method comprising using at least one computer hardware processor to perform obtaining RNA expression data for the subject, the RNA expression data indicating RNA expression levels for at least some genes in each group of at least some of a plurality of gene groups listed in Table 1; generating an RC TME signature for the subject using the RNA expression data, the RC TME signature comprising gene group scores for respective gene groups in the at least some of the plurality of gene groups, the generating comprising:

determining the gene group scores using the RNA expression levels; and identifying, using the RC TME signature and from among a plurality of RC TME types, an RC TME type for the subject.

In some aspects, the disclosure provides a method for determining a renal cancer (RC) myogenesis signature for a subject having, suspected of having, or at risk of having renal cancer, the method comprising using at least one computer hardware processor to perform obtaining RNA expression data for the subject, the RNA expression data indicating RNA expression levels for at least some of the genes in the gene group listed in Table 2; and generating a myogenesis signature for the subject using the RNA expression data, the myogenesis signature consisting of a gene group score for the gene group listed in Table 2, the gene group score determined using the RNA expression levels.

In some aspects, the disclosure provides a method for predicting the likelihood of a subject responding to an immuno-oncology (IO) agent, the subject having, suspected of having, or at risk of having renal cancer, the method comprising using at least one computer hardware processor to perform generating, using RNA expression data that has been obtained from a subject, a set of input features, the set of input features comprising at least two of the following features an RC TME type for the subject; RNA expression levels for one or more of the following genes: PD1, PD-L1, and PD-L2; an ECM associated signature for the subject; an Angiogenesis signature for the subject; a Proliferation rate signature for the subject; and a similarity score indicative of a similarity of an RC TME signature for the subject to RC TME signatures associated with RC TME type B and/or RC TME Type C samples; providing the set of input features as input to a machine learning model to obtain a corresponding output indicating a responder score, the responder score indicative of a likelihood that the subject responds to the immuno-oncology (IO) agent; identifying the subject as likely to have an increased likelihood of responding to the IO agent when the responder score is greater than a specified threshold.

In some aspects, the disclosure provides a method for predicting the likelihood of a subject responding to tyrosine kinase inhibitor (TKI), the subject having, suspected of having, or at risk of having renal cancer, the method comprising using at least one computer hardware processor to perform generating, using RNA expression data that has been obtained from a subject, a set of input features, the set of input features comprising at least two of the following features: a Macrophage signature for the subject; an Angiogenesis signature for the subject; a Proliferation rate signature for the subject; and a similarity score indicative of a similarity of an RC TME signature for the subject to RC TME signatures associated with RC TME type B samples; providing the set of input features as input to a machine learning model to

obtain a corresponding output indicating a responder score, the responder score indicative of a likelihood that the subject responds to the TKI; identifying the subject as likely to have an increased likelihood of responding to the TKI when the responder score is greater than a specified threshold.

In some aspects, the disclosure provides a method for identifying one or more therapeutic agents for administration to a subject having renal cancer, the method comprising: generating an International Metastatic RCC Database Consortium (IMDC) Risk Score for the subject; when the subject is identified as having a Poor IMDC Risk Score, identifying a combination of immuno-oncology (IO) agent and TKI as the one or more therapeutic agents for administration to the subject; when the subject is identified as having a Favorable or Intermediate IMDC Risk Score,

Generating an IO responder score according to a method as described herein; a TKI responder score according to a method as described herein; and identifying the one or more therapeutic agents for the subject using the IO responder score and the TKI responder score.

In some aspects, the disclosure provides a system, comprising at least one computer hardware processor; and at least one non-transitory computer readable medium storing processor-executable instructions that, when executed by the at least one computer hardware processor, causes the at least one computer hardware processor to perform a method for determining a renal cancer (RC) tumor microenvironment (TME) type for a subject, as described herein.

In some aspects, the disclosure provides at least one non-transitory computer readable medium storing processor-executable instructions that, when executed by at least one computer hardware processor, causes the at least one computer hardware processor to perform a method for determining a renal cancer (RC) tumor microenvironment (TME) type for a subject, as described herein.

In some aspects, the disclosure provides a system, comprising at least one computer hardware processor; and at least one non-transitory computer readable medium storing processor-executable instructions that, when executed by the at least one computer hardware processor, causes the at least one computer hardware processor to perform a method for determining a renal cancer (RC) myogenesis signature for a subject, as described herein.

In some aspects, the disclosure provides at least one non-transitory computer readable medium storing processor-executable instructions that, when executed by at least one computer hardware processor, causes the at least one computer hardware processor to perform a method for determining a renal cancer (RC) myogenesis signature for a subject, as described herein.

In some aspects, the disclosure provides a system, comprising at least one computer hardware processor; and at least one non-transitory computer readable medium storing processor-executable instructions that, when executed by the at least one computer hardware processor, causes the at least one computer hardware processor to perform a method for predicting the likelihood of a subject responding to an immuno-oncology (IO) agent, as described herein.

In some aspects, the disclosure provides at least one non-transitory computer readable medium storing processor-executable instructions that, when executed by at least one computer hardware processor, causes the at least one computer hardware processor to perform a method for predicting the likelihood of a subject responding to an immuno-oncology (IO) agent, as described herein.

In some aspects, the disclosure provides a system, comprising at least one computer hardware processor; and at least one non-transitory computer readable medium storing processor-executable instructions that, when executed by the at least one computer hardware processor, causes the at least one computer hardware processor to perform a method for predicting the likelihood of a subject responding to tyrosine kinase inhibitor (TKI), as described herein.

In some aspects, the disclosure provides at least one non-transitory computer readable medium storing processor-executable instructions that, when executed by at least one computer hardware processor, causes the at least one computer hardware processor to perform a method for predicting the likelihood of a subject responding to tyrosine kinase inhibitor (TKI), as described herein.

In some aspects, the disclosure provides a system, comprising at least one computer hardware processor; and at least one non-transitory computer readable medium storing processor-executable instructions that, when executed by the at least one computer hardware processor, causes the at least one computer hardware processor to perform a method for identifying one or more therapeutic agents for administration to a subject having renal cancer as described herein.

In some aspects, the disclosure provides at least one non-transitory computer readable medium storing processor-executable instructions that, when executed by at least one computer hardware processor, causes the at least one computer hardware processor to perform a method for identifying one or more therapeutic agents for administration to a subject having renal cancer as described herein.

In some embodiments, obtaining the RNA expression data for the subject comprises obtaining sequencing data previously obtained by sequencing a biological sample obtained from the subject.

In some embodiments, the sequencing data comprises at least 1 million reads, at least 5 million reads, at least 10 million reads, at least 20 million reads, at least 50 million reads, or at least 100 million reads. In some embodiments, the sequencing data comprises whole exome sequencing (WES) data, bulk RNA sequencing (RNA-seq) data, single cell RNA sequencing (scRNA-seq) data, or next generation sequencing (NGS) data. In some embodiments, the sequencing data comprises microarray data.

In some embodiments, the method further comprises normalizing the RNA expression data to transcripts per million (TPM) units prior to generating the RC TME signature.

In some embodiments, obtaining the RNA expression data for the subject comprises sequencing a biological sample obtained from the subject. In some embodiments, biological sample comprises kidney tissue of the subject. In some embodiments, the biological sample comprises tumor tissue of the subject.

In some embodiments, the RNA expression levels comprise RNA expression levels for at least three genes from each of at least two of the following gene groups: Effector cells group: PRF1, GZMB, TBX21, CD8B, ZAP70, IFNG, GZMK, EOMES, FASLG, CD8A, GZMA, GNLY; NK cells group: GZMB, NKG7, CD160, GZMH, CD244, EOMES, KLRK1, NCR1, GNLY, KLRF1, FGFBP2, SH2D1B, KIR2DL4, IFNG, NCR3, KLRC2, CD226; T cells group: TRAC, TRBC2, TBX21, CD3E, CD3D, ITK, TRBC1, CD3G, CD28, TRAT1, CD5; B cells group: CR2, MS4A1, CD79A, FCRL5, STAP1, TNFRSF17, TNFRSF13B, CD19, BLK, CD79B, TNFRSF13C, CD22, PAX5; Antitumor cytokines group: IFNA2, CCL3, TNF, TNFSF10, IL21, IFNB1; Checkpoint inhibition group: CTLA4, HAVCR2, CD274, LAG3, BTLA, VSIR, PDCD1LG2, TIGIT, PDCD1; Treg group: TNFRSF18, IKZF2, IL10, IKZF4, CTLA4, FOXP3, CCR8; Neutrophil signature group: FCGR3B, CD177, CTSG, PGLYRP1, FFAR2, CXCR2, PRTN3, ELANE, MPO, CXCR1; (i) Granulocyte traffic group: CXCL8, CCR3, CXCR2, CXCL2, CCL11, KITLG, CXCL1, CXCL5, CXCR1; MDSC group: ARG1, IL4I1, IL10, CYBB, IL6, PTGS2, IDO1; Macrophages group: MRC1, CD163, MSR1, SIGLEC1, IL4I1, CD68, IL10, CSF1R; Cancer-associated fibroblasts (CAF) group: PDGFRB, COL6A3, FBLN1, CXCL12, COL6A2, COL6A1, LUM, CD248, COL5A1, MMP2, COL1A1, MFAP5, PDGFRA, LRP1, FGF2, MMP3, FAP, COL1A2, ACTA2; Matrix group: COL11A1, LAMB3, FN1, COL1A1, COL4A1, ELN, LGALS9, LGALS7, LAMC2, TNC, LAMA3, COL3A1, COL5A1, VTN, COL1A2; Angiogenesis group: PGF, CXCL8, FLT1, ANGPT1, ANGPT2, VEGFC, VEGFB, CXCR2, VEGFA, VWF, CDH5, CXCL5, PDGFC, KDR, TEK; Endothelium group: NOS3, MMRN1, FLT1, CLEC14A, MMRN2, VCAM1, ENG, VWF, CDH5, KDR; Proliferation rate group: AURKA, MCM2, CCNB1, MYBL2, MCM6, CDK2, E2F1, CCNE1, ESCO2, CCND1, AURKB, BUB1, MKI67, PLK1, CETN3; EMT signature group: SNAI2, TWIST1, ZEB2, SNAI1, ZEB1, TWIST2, CDH2; Citric Acid Cycle group: ACLY, FAH, PC, MDH1B, SLC16A7, IREB2, PCK1, MDH1, SLC33A1, ALDH1B1, IDH3B, DLST, PDHB, MDH2, ACO1, IDH1, SLC5A6, HICDH, SLC16A8, GOT1, ME3, ME1, CS, OGDH, SDHA, ALDH5A1, CLYBL, SDHD, IDH3A, SLC25A1, ACSS2, SDHC, ACSS1, SUCLA2, SLC13A5, PDHX, SDHB, ALDH4A1, PCK2, DLD, ACO2, PDHA1, SLC13A2, FAHD1, IDH2, GOT2, ME2, ADSL, SUCLG2, SLC13A3, SUCLG1, SLC25A10, FH, IDH3G, SLC16A1, SLC25A11, PDHA2, DLAT; Glycolysis and Gluconeogenesis group: SLC2A9, PFKL, GCK, PFKFB4, SLC16A7, PCK1, PGAM2, GAPDH, BPGM, G6PC2, FBP2, LDHD, SLC2A3, GPI, ENO1, SLC25A11, PFKFB3, PFKM, LDHAL6B, SLC2A2, G6PC3, SLC2A6, GAPDHS, SLC2A11, PCK2, PFKP, PGK1, ALDOC, SLC2A10, ACYP2, SLC2A4, PKLR, HKDC1, PGK2, SLC2A8, PGAM1, SLC5A1, SLC5A12, SLC16A1, ALDOB, HK3, HK1, SLC5A9, GPD2, PFKFB1, SLC2A7, SLC5A11, SLC5A3, ACYP1, SLC16A8, PFKFB2, ALDOA, SLC5A2, HK2, ENO3, SLC2A12, FBP1, LDHA, LDHB, LDHC, G6PC, SLC2A14, SLC5A8, TPI1, SLC16A3, PKM2, ENO2, PGM1, UEVLD, LDHAL6A, SLC2A1, PGM2; and Fatty Acid Metabolism group: MLYCD, ALDH3A2, SLC27A5, SLC27A3, LIPC, SLC27A2, ACSL4, ACSL1, PCCB, SLC25A20, AADAC, SLC22A4, SLC22A5, ECH1, PCCA, SLC27A1, SLC27A4, CROT, ACSL5, ACSL3, CYP4F12.

In some embodiments, the RNA expression levels comprise RNA expression levels for at least three genes from each of at least two of the following gene groups: MHC I group: HLA-C, B2M, HLA-B, HLA-A, TAP1, TAP2, NLRC5, TAPBP; MHC II group: HLA-DQA1, HLA-DMA, HLA-DRB1, HLA-DMB, CIITA, HLA-DPA1, HLA-DPB1, HLA-DRA, HLA-DQB1; Coactivation molecules group: CD80, TNFRSF4, CD27, CD83, TNFSF9, CD40LG, CD70, ICOS, CD86, CD40, TNFSF4, ICOSLG, TNFRSF9, CD28; Effector cells group: PRF1, GZMB, TBX21, CD8B, ZAP70, IFNG, GZMK, EOMES, FASLG, CD8A, GZMA, GNLY; T cell traffic group: CXCL9, CCL3, CXCR3, CXCL10, CXCL11, CCL5, CCL4, CX3CL1, CX3CR1; NK cells group: GZMB, NKG7, CD160, GZMH, CD244, EOMES, KLRK1, NCR1, GNLY, KLRF1, FGFBP2, SH2D1B, KIR2DL4, IFNG, NCR3, KLRC2, CD226; T cells group: TRAC, TRBC2, TBX21, CD3E, CD3D, ITK, TRBC1, CD3G, CD28, TRAT1, CD5; B cells group: CR2, MS4A1, CD79A, FCRL5, STAP1, TNFRSF17, TNFRSF13B, CD19, BLK, CD79B, TNFRSF13C, CD22, PAX5; M1 signatures group: IL1B, IL12B, NOS2, SOCS3, IRF5, IL23A, TNF, IL12A, CMKLR1; Th1 signature group: IL12RB2, IL2, TBX21, IFNG, STAT4, IL21, CD40LG; Antitumor cytokines group: IFNA2, CCL3, TNF, TNFSF10, IL21, IFNB1; Checkpoint inhibition group: CTLA4, HAVCR2, CD274, LAG3, BTLA, VSIR, PDCD1LG2, TIGIT, PDCD1; Treg group: TNFRSF18, IKZF2, IL10, IKZF4, CTLA4, FOXP3, CCR8; T reg traffic group: CCL28, CCR10, CCR4, CCR8, CCL17, CCL22, CCL1; Neutrophil signature group: FCGR3B, CD177, CTSG, PGLYRP1, FFAR2, CXCR2, PRTN3, ELANE, MPO, CXCR1; Granulocyte traffic group: CXCL8, CCR3, CXCR2, CXCL2, CCL11, KITLG, CXCL1, CXCL5, CXCR1; MDSC group: ARG1, IL4I1, IL10, CYBB, IL6, PTGS2, IDO1; MDSC traffic group: CCL15, IL6R, CSF2RA, CSF2, CXCL8, CXCL12, IL6, CSF3, CCL26, CXCR4, CXCR2, CSF3R, CSF1, CXCL5, CSF1R; Macrophages group: MRC1, CD163, MSR1, SIGLEC1, IL4I1, CD68, IL10, CSF1R; Macrophage DC traffic group: CCL7, CCL2, XCR1, XCL1, CSF1, CCR2, CCL8, CSF1R; Th2 signature group: IL13, CCR4, IL10, IL5, IL4; Protumor cytokines group: MIF, TGFB1, IL10, TGFB3, IL6, TGFB2, IL22; CAF group: PDGFRB, COL6A3, FBLN1, CXCL12, COL6A2, COL6A1, LUM, CD248, COL5A1, MMP2, COL1A1, MFAP5, PDGFRA, LRP1, FGF2, MMP3, FAP, COL1A2, ACTA2; Matrix group: COL11A1, LAMB3, FN1, COL1A1, COL4A1, ELN, LGALS9, LGALS7, LAMC2, TNC, LAMA3, COL3A1, COL5A1, VTN, COL1A2; Matrix remodeling group: MMP1, PLOD2, MMP2, MMP12, ADAMTS5, ADAMTS4, LOX, MMP9, MMP11, MMP3, MMP1, CA9; Angiogenesis group: PGF, CXCL8, FLT1, ANGPT1, ANGPT2, VEGFC, VEGFB, CXCR2, VEGFA, VWF, CDH5, CXCL5, PDGFC, KDR, TEK; Endothelium group: NOS3, MMRN1, FLT1, CLEC14A, MMRN2, VCAM1, ENG, VWF, CDH5, KDR; Proliferation rate group: AURKA, MCM2, CCNB1, MYBL2, MCM6, CDK2, E2F1, CCNE1, ESCO2, CCND1, AURKB, BUB1, MKI67, PLK1, CETN3; EMT signature group: SNAI2, TWIST1, ZEB2, SNAI1, ZEB1, TWIST2, CDH2; Cyclic Nucleotides Metabolism group: ADCY4, PDE11A, PDE6A, PDE9A, PDE6C, ADCY7, PDE4A, PDE8A, PDE1B, PDE1A, GUCY2C, GUCY1A3, ADCY9, ADCY2, PDE6B, ADCY8, PDE8B, GUCY2F, PDE4C, PDE3A, GUCY1A2, PDE6G, PDE1C, GUCY2D, ADCY10, GUCY1B3, GUCY1B2, PDE7B, PDE5A, PDE6D, NPR2, ADCY5, NPR1, ADCY6, PDE7A, PDE2A, PDE4B, PDE10A, PDE6H, PDE4D, ADCY1, PDE3B, ADCY3; Glycolysis and Gluconeogenesis group: SLC2A9, PFKL, GCK, PFKFB4, SLC16A7, PCK1, PGAM2, GAPDH, BPGM, G6PC2, FBP2, LDHD, SLC2A3, GPI, ENO1, SLC25A11, PFKFB3, PFKM, LDHAL6B, SLC2A2, G6PC3, SLC2A6, GAPDHS, SLC2A11, PCK2, PFKP, PGK1, ALDOC, SLC2A10, ACYP2, SLC2A4, PKLR, HKDC1, PGK2, SLC2A8, PGAM1, SLC5A1, SLC5A12, SLC16A1, ALDOB, HK3, HK1, SLC5A9, GPD2, PFKFB1, SLC2A7, SLC5A11, SLC5A3, ACYP1, SLC16A8, PFKFB2, ALDOA, SLC5A2, HK2, ENO3, SLC2A12, FBP1, LDHA, LDHB, LDHC, G6PC, SLC2A14, SLC5A8, TPI1, SLC16A3, PKM2, ENO2, PGM1, UEVLD, LDHAL6A, SLC2A1, PGM2; Citric Acid Cycle group: ACLY, FAH, PC, MDH1B, SLC16A7, IREB2, PCK1, MDH1, SLC33A1, ALDH1B1, IDH3B, DLST, PDHB, MDH2, ACO1, IDH1, SLC5A6, HICDH, SLC16A8, GOT1, ME3, ME1, CS, OGDH, SDHA, ALDH5A1, CLYBL, SDHD, IDH3A, SLC25A1, ACSS2, SDHC, ACSS1, SUCLA2, SLC13A5, PDHX, SDHB, ALDH4A1, PCK2, DLD, ACO2, PDHA1, SLC13A2, FAHD1, IDH2, GOT2, ME2, ADSL, SUCLG2, SLC13A3, SUCLG1, SLC25A10, FH, IDH3G, SLC16A1, SLC25A11, PDHA2, DLAT; and, Fatty Acid Metabolism group: MLYCD, ALDH3A2, SLC27A5, SLC27A3, LIPC, SLC27A2, ACSL4, ACSL1, PCCB, SLC25A20, AADAC, SLC22A4, SLC22A5, ECH1, PCCA, SLC27A1, SLC27A4, CROT, ACSL5, ACSL3, CYP4F12.

In some embodiments, the RNA expression levels further comprise RNA expression levels for at least three genes from each of at least two of the following gene groups: ECM associated group: ADAM8, ADAMTS4, C1QL3, CST7, CTSW, CXCL8, FASLG, LTB, MUC1, OSM, P4HA2, SCUBE1, SEMA4B, SEMA7A, SERPINE1, TCHH, TGFA, TGM2, TNFSF11, TNFSF9, WNT10B; TLS kidney group: ZNF683, POU2AF1, LAX1, CD79A, CXCL9, XCL2, JCHAIN, SLAMF7, CD38, SLAMF1, TNFRSF17, IRF4, HSH2D, PLA2G2D, MZB1; NRF2 signature group: TRIM16L, UGDH, KIAA1549, PANX2, FECH, LRP8, AKR1C2, FTH1, AKR1C3, CBR1, PFN2, CBX2, TXN, CYP4F11, CYP4F3, AKR1C1, AKR1B15, G6PD, PRDX1, TALDO1, EPT1, SRXN1, JAKMIP3, FTHL3, UCHL1, TXNRD1, Clorf131, CASKIN1, PGD, GPX2, OSGIN1, KIAA0319, CABYR, AIFM2, TRIM16, AKR1B10, GCLC, ABCC2, ETFB, IDH1, MAFG, NECAB2, ME1, PTGR1, PIR, GSR, RIT1, GCLM, ALDH3A1, NQO1, PKD1L2, NRG4, ABHD4, HRG, SLC7A11; and, tRCC signature group: FST, TRIM63, SLC10A2, ANTXRL, ERW-2, SNX22, INHBE, SV2B, FAM124A, EPHA5, LUZP2, CPEB1, HOXB13, ALLC, KCNF1, NDRG4, GREB1, ASTN1, JSRP1, UBE2U, KCNQ4, MYO7B, BRINP2, C1QL2, CCDC136, SLC51B, CATSPERG, PMEL, BIRC7, PLK5, ADARB2, CFAP61, TUBB4A, PLIN4, ABCB5, SYT3, HCN4, CTSK, SPACA1, TRIM67, NMRK2, LGI3, ARHGEF4, NTSR2, KEL, SNCB, PLD5, ADGRB1, CYP17A1, IGFBPL1, TRIM71, SLC45A2, TP73, IP6K3, HABP2, RGS20, IGFN1, CDH17.

In some embodiments, the RNA expression levels comprise RNA expression levels for each of the genes from each of the following gene groups: Effector cells group: PRF1, GZMB, TBX21, CD8B, ZAP70, IFNG, GZMK, EOMES, FASLG, CD8A, GZMA, GNLY; NK cells group: GZMB, NKG7, CD160, GZMH, CD244, EOMES, KLRK1, NCR1, GNLY, KLRF1, FGFBP2, SH2D1B, KIR2DL4, IFNG, NCR3, KLRC2, CD226; T cells group: TRAC, TRBC2, TBX21, CD3E, CD3D, ITK, TRBC1, CD3G, CD28, TRAT1, CD5; B cells group: CR2, MS4A1, CD79A, FCRL5, STAP1, TNFRSF17, TNFRSF13B, CD19, BLK, CD79B, TNFRSF13C, CD22, PAX5; Antitumor cytokines group: IFNA2, CCL3, TNF, TNFSF10, IL21, IFNB1; Checkpoint inhibition group: CTLA4, HAVCR2, CD274, LAG3, BTLA, VSIR, PDCD1LG2, TIGIT, PDCD1; Treg group: TNFRSF18, IKZF2, IL10, IKZF4, CTLA4, FOXP3, CCR8; Neutrophil signature group: FCGR3B, CD177, CTSG, PGLYRP1, FFAR2, CXCR2, PRTN3, ELANE, MPO, CXCR1; Granulocyte traffic group: CXCL8, CCR3, CXCR2, CXCL2, CCL11, KITLG, CXCL1, CXCL5, CXCR1; MDSC group: ARG1, IL4I1, IL10, CYBB, IL6, PTGS2, IDO1; Macrophages group: MRC1, CD163, MSR1, SIGLEC1, IL4I1, CD68, IL10, CSF1R; Cancer-associated fibroblasts (CAF) group: PDGFRB, COL6A3, FBLN1, CXCL12, COL6A2, COL6A1, LUM, CD248, COL5A1, MMP2, COL1A1, MFAP5, PDGFRA, LRP1, FGF2, MMP3, FAP, COL1A2, ACTA2; Matrix group: COL11A1, LAMB3, FN1, COL1A1, COL4A1, ELN, LGALS9, LGALS7, LAMC2, TNC, LAMA3, COL3A1, COL5A1, VTN, COL1A2; Angiogenesis group: PGF, CXCL8, FLT1, ANGPT1, ANGPT2, VEGFC, VEGFB, CXCR2, VEGFA, VWF, CDH5, CXCL5, PDGFC, KDR, TEK; Endothelium group: NOS3, MMRN1, FLT1, CLEC14A, MMRN2, VCAM1, ENG, VWF, CDH5, KDR; Proliferation rate group: AURKA, MCM2, CCNB1, MYBL2, MCM6, CDK2, E2F1, CCNE1, ESCO2, CCND1, AURKB, BUB1, MKI67, PLK1, CETN3; EMT signature group: SNAI2, TWIST1, ZEB2, SNAI1, ZEB1, TWIST2, CDH2; Citric Acid Cycle group: ACLY, FAH, PC, MDH1B, SLC16A7, IREB2, PCK1, MDH1, SLC33A1, ALDH1B1, IDH3B, DLST, PDHB, MDH2, ACO1, IDH1, SLC5A6, HICDH, SLC16A8, GOT1, ME3, ME1, CS, OGDH, SDHA, ALDH5A1, CLYBL, SDHD, IDH3A, SLC25A1, ACSS2, SDHC, ACSS1, SUCLA2, SLC13A5, PDHX, SDHB, ALDH4A1, PCK2, DLD, ACO2, PDHA1, SLC13A2, FAHD1, IDH2, GOT2, ME2, ADSL, SUCLG2, SLC13A3, SUCLG1, SLC25A10, FH, IDH3G, SLC16A1, SLC25A11, PDHA2, DLAT; Glycolysis and Gluconeogenesis group: SLC2A9, PFKL, GCK, PFKFB4, SLC16A7, PCK1, PGAM2, GAPDH, BPGM, G6PC2, FBP2, LDHD, SLC2A3, GPI, ENO1, SLC25A11, PFKFB3, PFKM, LDHAL6B, SLC2A2, G6PC3, SLC2A6, GAPDHS, SLC2A11, PCK2, PFKP, PGK1, ALDOC, SLC2A10, ACYP2, SLC2A4, PKLR, HKDC1, PGK2, SLC2A8, PGAM1, SLC5A1, SLC5A12, SLC16A1, ALDOB, HK3, HK1, SLC5A9, GPD2, PFKFB1, SLC2A7, SLC5A11, SLC5A3, ACYP1, SLC16A8, PFKFB2, ALDOA, SLC5A2, HK2, ENO3, SLC2A12, FBP1, LDHA, LDHB, LDHC, G6PC, SLC2A14, SLC5A8, TPI1, SLC16A3, PKM2, ENO2, PGM1, UEVLD, LDHAL6A, SLC2A1, PGM2; and Fatty Acid Metabolism group: MLYCD, ALDH3A2, SLC27A5, SLC27A3, LIPC, SLC27A2, ACSL4, ACSL1, PCCB, SLC25A20, AADAC, SLC22A4, SLC22A5, ECH1, PCCA, SLC27A1, SLC27A4, CROT, ACSL5, ACSL3, CYP4F12.

In some embodiments, the RNA expression levels for genes in the plurality of gene groups comprise RNA expression levels for each of the genes from each of the following gene groups: MHC I group: HLA-C, B2M, HLA-B, HLA-A, TAP1, TAP2, NLRC5, TAPBP; MHC II group: HLA-DQA1, HLA-DMA, HLA-DRB1, HLA-DMB, CIITA, HLA-DPA1, HLA-DPB1, HLA-DRA, HLA-DQB1; Coactivation molecules group: CD80, TNFRSF4, CD27, CD83, TNFSF9, CD40LG, CD70, ICOS, CD86, CD40, TNFSF4, ICOSLG, TNFRSF9, CD28; Effector cells group: PRF1, GZMB, TBX21, CD8B, ZAP70, IFNG, GZMK, EOMES, FASLG, CD8A, GZMA, GNLY; T cell traffic group: CXCL9, CCL3, CXCR3, CXCL10, CXCL11, CCL5, CCL4, CX3CL1, CX3CR1; NK cells group: GZMB, NKG7, CD160, GZMH, CD244, EOMES, KLRK1, NCR1, GNLY, KLRF1, FGFBP2, SH2D1B, KIR2DL4, IFNG, NCR3, KLRC2, CD226; T cells group: TRAC, TRBC2, TBX21, CD3E, CD3D, ITK, TRBC1, CD3G, CD28, TRAT1, CD5; B cells group: CR2, MS4A1, CD79A, FCRL5, STAP1, TNFRSF17, TNFRSF13B, CD19, BLK, CD79B, TNFRSF13C, CD22, PAX5; M1 signatures group: IL1B, IL12B, NOS2, SOCS3, IRF5, IL23A, TNF, IL12A, CMKLR1; Th1 signature group: IL12RB2, IL2, TBX21, IFNG, STAT4, IL21, CD40LG; Antitumor cytokines group: IFNA2, CCL3, TNF, TNFSF10, IL21, IFNB1; Checkpoint inhibition group: CTLA4, HAVCR2, CD274, LAG3, BTLA, VSIR, PDCD1LG2, TIGIT, PDCD1; Treg group: TNFRSF18, IKZF2, IL10, IKZF4, CTLA4, FOXP3, CCR8; T reg traffic group: CCL28, CCR10, CCR4, CCR8, CCL17, CCL22, CCL1; Neutrophil signature group: FCGR3B, CD177, CTSG, PGLYRP1, FFAR2, CXCR2, PRTN3, ELANE, MPO, CXCR1; Granulocyte traffic group: CXCL8, CCR3, CXCR2, CXCL2, CCL11, KITLG, CXCL1, CXCL5, CXCR1; MDSC group: ARG1, IL4I1, IL10, CYBB, IL6, PTGS2, IDO1; MDSC traffic group: CCL15, IL6R, CSF2RA, CSF2, CXCL8, CXCL12, IL6, CSF3, CCL26, CXCR4, CXCR2, CSF3R, CSF1, CXCL5, CSF1R; Macrophages group: MRC1, CD163, MSR1, SIGLEC1, IL4I1, CD68, IL10, CSF1R; Macrophage DC traffic group: CCL7, CCL2, XCR1, XCL1, CSF1, CCR2, CCL8, CSF1R; Th2 signature group: IL13, CCR4, IL10, IL5, IL4; Protumor cytokines group: MIF, TGFB1, IL10, TGFB3, IL6, TGFB2, IL22; CAF group: PDGFRB, COL6A3, FBLN1, CXCL12, COL6A2, COL6A1, LUM, CD248, COL5A1, MMP2, COL1A1, MFAP5, PDGFRA, LRP1, FGF2, MMP3, FAP, COL1A2, ACTA2; Matrix group: COL11A1, LAMB3, FN1, COL1A1, COL4A1, ELN, LGALS9, LGALS7, LAMC2, TNC, LAMA3, COL3A1, COL5A1, VTN, COL1A2; Matrix remodeling group: MMP1, PLOD2, MMP2, MMP12, ADAMTS5, ADAMTS4, LOX, MMP9, MMP11, MMP3, MMP1, CA9; Angiogenesis group: PGF, CXCL8, FLT1, ANGPT1, ANGPT2, VEGFC, VEGFB, CXCR2, VEGFA, VWF, CDH5, CXCL5, PDGFC, KDR, TEK; Endothelium group: NOS3, MMRN1, FLT1, CLEC14A, MMRN2, VCAM1, ENG, VWF, CDH5, KDR; Proliferation rate group: AURKA, MCM2, CCNB1, MYBL2, MCM6, CDK2, E2F1, CCNE1, ESCO2, CCND1, AURKB, BUB1, MKI67, PLK1, CETN3; EMT signature group: SNAI2, TWIST1, ZEB2, SNAI1, ZEB1, TWIST2, CDH2; Cyclic Nucleotides Metabolism group: ADCY4, PDE11A, PDE6A, PDE9A, PDE6C, ADCY7, PDE4A, PDE8A, PDE1B, PDE1A, GUCY2C, GUCY1A3, ADCY9, ADCY2, PDE6B, ADCY8, PDE8B, GUCY2F, PDE4C, PDE3A, GUCY1A2, PDE6G, PDE1C, GUCY2D, ADCY10, GUCY1B3, GUCY1B2, PDE7B, PDE5A, PDE6D, NPR2, ADCY5, NPR1, ADCY6, PDE7A, PDE2A, PDE4B, PDE10A, PDE6H, PDE4D, ADCY1, PDE3B, ADCY3; Glycolysis and Gluconeogenesis group: SLC2A9, PFKL, GCK, PFKFB4, SLC16A7, PCK1, PGAM2, GAPDH, BPGM, G6PC2, FBP2, LDHD, SLC2A3, GPI, ENO1, SLC25A11, PFKFB3, PFKM, LDHAL6B, SLC2A2, G6PC3, SLC2A6, GAPDHS, SLC2A11, PCK2, PFKP, PGK1, ALDOC, SLC2A10, ACYP2, SLC2A4, PKLR, HKDC1, PGK2, SLC2A8, PGAM1, SLC5A1, SLC5A12, SLC16A1, ALDOB, HK3, HK1, SLC5A9, GPD2, PFKFB1, SLC2A7, SLC5A11, SLC5A3, ACYP1, SLC16A8, PFKFB2, ALDOA, SLC5A2, HK2, ENO3, SLC2A12, FBP1, LDHA, LDHB, LDHC, G6PC, SLC2A14, SLC5A8, TPI1, SLC16A3, PKM2, ENO2, PGM1, UEVLD, LDHAL6A, SLC2A1, PGM2; Citric Acid Cycle group: ACLY, FAH, PC, MDH1B, SLC16A7, IREB2, PCK1, MDH1, SLC33A1, ALDH1B1, IDH3B, DLST, PDHB, MDH2, ACO1, IDH1, SLC5A6, HICDH, SLC16A8, GOT1, ME3, ME1, CS, OGDH, SDHA, ALDH5A1, CLYBL, SDHD, IDH3A, SLC25A1, ACSS2, SDHC, ACSS1, SUCLA2, SLC13A5, PDHX, SDHB, ALDH4A1, PCK2, DLD, ACO2, PDHA1, SLC13A2, FAHD1, IDH2, GOT2, ME2, ADSL, SUCLG2, SLC13A3, SUCLG1, SLC25A10, FH, IDH3G, SLC16A1, SLC25A11, PDHA2, DLAT; and, Fatty Acid Metabolism group: MLYCD, ALDH3A2, SLC27A5, SLC27A3, LIPC, SLC27A2, ACSL4, ACSL1, PCCB, SLC25A20, AADAC, SLC22A4, SLC22A5, ECH1, PCCA, SLC27A1, SLC27A4, CROT, ACSL5, ACSL3, CYP4F12.

In some embodiments, the RNA expression levels for genes in the plurality of gene groups further comprise RNA expression levels for each of the genes from each of the following gene groups: ECM associated group: ADAM8, ADAMTS4, C1QL3, CST7, CTSW, CXCL8, FASLG, LTB, MUC1, OSM, P4HA2, SCUBE1, SEMA4B, SEMA7A, SERPINE1, TCHH, TGFA, TGM2, TNFSF11, TNFSF9, WNT10B; TLS kidney group: ZNF683, POU2AF1, LAX1, CD79A, CXCL9, XCL2, JCHAIN, SLAMF7, CD38, SLAMF1, TNFRSF17, IRF4, HSH2D, PLA2G2D, MZB1; NRF2 signature group: TRIM16L, UGDH, KIAA1549, PANX2, FECH, LRP8, AKR1C2, FTH1, AKR1C3, CBR1, PFN2, CBX2, TXN, CYP4F11, CYP4F3, AKR1C1, AKR1B15, G6PD, PRDX1, TALDO1, EPT1, SRXN1, JAKMIP3, FTHL3, UCHL1, TXNRD1, Clorf131, CASKIN1, PGD, GPX2, OSGIN1, KIAA0319, CABYR, AIFM2, TRIM16, AKR1B10, GCLC, ABCC2, ETFB, IDH1, MAFG, NECAB2, ME1, PTGR1, PIR, GSR, RIT1, GCLM, ALDH3A1, NQO1, PKD1L2, NRG4, ABHD4, HRG, SLC7A11; and, tRCC signature group: FST, TRIM63, SLC10A2, ANTXRL, ERW-2, SNX22, INHBE, SV2B, FAM124A, EPHA5, LUZP2, CPEB1, HOXB13, ALLC, KCNF1, NDRG4, GREB1, ASTN1, JSRP1, UBE2U, KCNQ4, MYO7B, BRINP2, C1QL2, CCDC136, SLC51B, CATSPERG, PMEL, BIRC7, PLK5, ADARB2, CFAP61, TUBB4A, PLIN4, ABCB5, SYT3, HCN4, CTSK, SPACA1, TRIM67, NMRK2, LGI3, ARHGEF4, NTSR2, KEL, SNCB, PLD5, ADGRB1, CYP17A1, IGFBPL1, TRIM71, SLC45A2, TP73, IP6K3, HABP2, RGS20, IGFN1, CDH17.

In some embodiments, determining the gene group scores comprises determining a respective gene group score for each of at least two of the following gene groups, using, for a particular gene group, RNA expression levels for at least three genes in the particular gene group to determine the gene group score for the particular group, the gene groups including: Effector cells group: PRF1, GZMB, TBX21, CD8B, ZAP70, IFNG, GZMK, EOMES, FASLG, CD8A, GZMA, GNLY; NK cells group: GZMB, NKG7, CD160, GZMH, CD244, EOMES, KLRK1, NCR1, GNLY, KLRF1, FGFBP2, SH2D1B, KIR2DL4, IFNG, NCR3, KLRC2, CD226; T cells group: TRAC, TRBC2, TBX21, CD3E, CD3D, ITK, TRBC1, CD3G, CD28, TRAT1, CD5; B cells group: CR2, MS4A1, CD79A, FCRL5, STAP1, TNFRSF17, TNFRSF13B, CD19, BLK, CD79B, TNFRSF13C, CD22, PAX5; Antitumor cytokines group: IFNA2, CCL3, TNF, TNFSF10, IL21, IFNB1; Checkpoint inhibition group: CTLA4, HAVCR2, CD274, LAG3, BTLA, VSIR, PDCD1LG2, TIGIT, PDCD1; Treg group: TNFRSF18, IKZF2, IL10, IKZF4, CTLA4, FOXP3, CCR8; Neutrophil signature group: FCGR3B, CD177, CTSG, PGLYRP1, FFAR2, CXCR2, PRTN3, ELANE, MPO, CXCR1; Granulocyte traffic group: CXCL8, CCR3, CXCR2, CXCL2, CCL11, KITLG, CXCL1, CXCL5, CXCR1; MDSC group: ARG1, IL4I1, IL10, CYBB, IL6, PTGS2, IDO1; Macrophages group: MRC1, CD163, MSR1, SIGLEC1, IL4I1, CD68, IL10, CSF1R; Cancer-associated fibroblasts (CAF) group: PDGFRB, COL6A3, FBLN1, CXCL12, COL6A2, COL6A1, LUM, CD248, COL5A1, MMP2, COL1A1, MFAP5, PDGFRA, LRP1, FGF2, MMP3, FAP, COL1A2, ACTA2; Matrix group: COL11A1, LAMB3, FN1, COL1A1, COL4A1, ELN, LGALS9, LGALS7, LAMC2, TNC, LAMA3, COL3A1, COL5A1, VTN, COL1A2; Angiogenesis group: PGF, CXCL8, FLT1, ANGPT1, ANGPT2, VEGFC, VEGFB, CXCR2, VEGFA, VWF, CDH5, CXCL5, PDGFC, KDR, TEK; Endothelium group: NOS3, MMRN1, FLT1, CLEC14A, MMRN2, VCAM1, ENG, VWF, CDH5, KDR; Proliferation rate group: AURKA, MCM2, CCNB1, MYBL2, MCM6, CDK2, E2F1, CCNE1, ESCO2, CCND1, AURKB, BUB1, MKI67, PLK1, CETN3; EMT signature group: SNAI2, TWIST1, ZEB2, SNAI1, ZEB1, TWIST2, CDH2; Citric Acid Cycle group: ACLY, FAH, PC, MDH1B, SLC16A7, IREB2, PCK1, MDH1, SLC33A1, ALDH1B1, IDH3B, DLST, PDHB, MDH2, ACO1, IDH1, SLC5A6, HICDH, SLC16A8, GOT1, ME3, ME1, CS, OGDH, SDHA, ALDH5A1, CLYBL, SDHD, IDH3A, SLC25A1, ACSS2, SDHC, ACSS1, SUCLA2, SLC13A5, PDHX, SDHB, ALDH4A1, PCK2, DLD, ACO2, PDHA1, SLC13A2, FAHD1, IDH2, GOT2, ME2, ADSL, SUCLG2, SLC13A3, SUCLG1, SLC25A10, FH, IDH3G, SLC16A1, SLC25A11, PDHA2, DLAT; Glycolysis and Gluconeogenesis group: SLC2A9, PFKL, GCK, PFKFB4, SLC16A7, PCK1, PGAM2, GAPDH, BPGM, G6PC2, FBP2, LDHD, SLC2A3, GPI, ENO1, SLC25A11, PFKFB3, PFKM, LDHAL6B, SLC2A2, G6PC3, SLC2A6, GAPDHS, SLC2A11, PCK2, PFKP, PGK1, ALDOC, SLC2A10, ACYP2, SLC2A4, PKLR, HKDC1, PGK2, SLC2A8, PGAM1, SLC5A1, SLC5A12, SLC16A1, ALDOB, HK3, HK1, SLC5A9, GPD2, PFKFB1, SLC2A7, SLC5A11, SLC5A3, ACYP1, SLC16A8, PFKFB2, ALDOA, SLC5A2, HK2, ENO3, SLC2A12, FBP1, LDHA, LDHB, LDHC, G6PC, SLC2A14, SLC5A8, TPI1, SLC16A3, PKM2, ENO2, PGM1, UEVLD, LDHAL6A, SLC2A1, PGM2; and Fatty Acid Metabolism group: MLYCD, ALDH3A2, SLC27A5, SLC27A3, LIPC, SLC27A2, ACSL4, ACSL1, PCCB, SLC25A20, AADAC, SLC22A4, SLC22A5, ECH1, PCCA, SLC27A1, SLC27A4, CROT, ACSL5, ACSL3, CYP4F12.

In some embodiments, determining the gene group scores comprises determining a respective gene group score for each of at least two of the following gene groups, using, for a particular gene group, RNA expression levels for at least three genes in the particular gene group to determine the gene group score for the particular group, the gene groups including: MHC I group: HLA-C, B2M, HLA-B, HLA-A, TAP1, TAP2, NLRC5, TAPBP; MHC II group: HLA-DQA1, HLA-DMA, HLA-DRB1, HLA-DMB, CIITA, HLA-DPA1, HLA-DPB1, HLA-DRA, HLA-DQB1; Coactivation molecules group: CD80, TNFRSF4, CD27, CD83, TNFSF9, CD40LG, CD70, ICOS, CD86, CD40, TNFSF4, ICOSLG, TNFRSF9, CD28; Effector cells group: PRF1, GZMB, TBX21, CD8B, ZAP70, IFNG, GZMK, EOMES, FASLG, CD8A, GZMA, GNLY; T cell traffic group: CXCL9, CCL3, CXCR3, CXCL10, CXCL11, CCL5, CCL4, CX3CL1, CX3CR1; NK cells group: GZMB, NKG7, CD160, GZMH, CD244, EOMES, KLRK1, NCR1, GNLY, KLRF1, FGFBP2, SH2D1B, KIR2DL4, IFNG, NCR3, KLRC2, CD226; T cells group: TRAC, TRBC2, TBX21, CD3E, CD3D, ITK, TRBC1, CD3G, CD28, TRAT1, CD5; B cells group: CR2, MS4A1, CD79A, FCRL5, STAP1, TNFRSF17, TNFRSF13B, CD19, BLK, CD79B, TNFRSF13C, CD22, PAX5; M1 signatures group: IL1B, IL12B, NOS2, SOCS3, IRF5, IL23A, TNF, IL12A, CMKLR1; Th1 signature group: IL12RB2, IL2, TBX21, IFNG, STAT4, IL21, CD40LG; Antitumor cytokines group: IFNA2, CCL3, TNF, TNFSF10, IL21, IFNB1; Checkpoint inhibition group: CTLA4, HAVCR2, CD274, LAG3, BTLA, VSIR, PDCD1LG2, TIGIT, PDCD1; Treg group: TNFRSF18, IKZF2, IL10, IKZF4, CTLA4, FOXP3, CCR8; T reg traffic group: CCL28, CCR10, CCR4, CCR8, CCL17, CCL22, CCL1; Neutrophil signature group: FCGR3B, CD177, CTSG, PGLYRP1, FFAR2, CXCR2, PRTN3, ELANE, MPO, CXCR1; Granulocyte traffic group: CXCL8, CCR3, CXCR2, CXCL2, CCL11, KITLG, CXCL1, CXCL5, CXCR1; MDSC group: ARG1, IL4I1, IL10, CYBB, IL6, PTGS2, IDO1; MDSC traffic group: CCL15, IL6R, CSF2RA, CSF2, CXCL8, CXCL12, IL6, CSF3, CCL26, CXCR4, CXCR2, CSF3R, CSF1, CXCL5, CSF1R; Macrophages group: MRC1, CD163, MSR1, SIGLEC1, IL4I1, CD68, IL10, CSF1R; Macrophage DC traffic group: CCL7, CCL2, XCR1, XCL1, CSF1, CCR2, CCL8, CSF1R; Th2 signature group: IL13, CCR4, IL10, IL5, IL4; Protumor cytokines group: MIF, TGFB1, IL10, TGFB3, IL6, TGFB2, IL22; CAF group: PDGFRB, COL6A3, FBLN1, CXCL12, COL6A2, COL6A1, LUM, CD248, COL5A1, MMP2, COL1A1, MFAP5, PDGFRA, LRP1, FGF2, MMP3, FAP, COL1A2, ACTA2; Matrix group: COL11A1, LAMB3, FN1, COL1A1, COL4A1, ELN, LGALS9, LGALS7, LAMC2, TNC, LAMA3, COL3A1, COL5A1, VTN, COL1A2; Matrix remodeling group: MMP1, PLOD2, MMP2, MMP12, ADAMTS5, ADAMTS4, LOX, MMP9, MMP11, MMP3, MMP1, CA9; Angiogenesis group: PGF, CXCL8, FLT1, ANGPT1, ANGPT2, VEGFC, VEGFB, CXCR2, VEGFA, VWF, CDH5, CXCL5, PDGFC, KDR, TEK; Endothelium group: NOS3, MMRN1, FLT1, CLEC14A, MMRN2, VCAM1, ENG, VWF, CDH5, KDR; Proliferation rate group: AURKA, MCM2, CCNB1, MYBL2, MCM6, CDK2, E2F1, CCNE1, ESCO2, CCND1, AURKB, BUB1, MKI67, PLK1, CETN3; EMT signature group: SNAI2, TWIST1, ZEB2, SNAI1, ZEB1, TWIST2, CDH2; Cyclic Nucleotides Metabolism group: ADCY4, PDE11A, PDE6A, PDE9A, PDE6C, ADCY7, PDE4A, PDE8A, PDE1B, PDE1A, GUCY2C, GUCY1A3, ADCY9, ADCY2, PDE6B, ADCY8, PDE8B, GUCY2F, PDE4C, PDE3A, GUCY1A2, PDE6G, PDE1C, GUCY2D, ADCY10, GUCY1B3, GUCY1B2, PDE7B, PDE5A, PDE6D, NPR2, ADCY5, NPR1, ADCY6, PDE7A, PDE2A, PDE4B, PDE10A, PDE6H, PDE4D, ADCY1, PDE3B, ADCY3; Glycolysis and Gluconeogenesis group: SLC2A9, PFKL, GCK, PFKFB4, SLC16A7, PCK1, PGAM2, GAPDH, BPGM, G6PC2, FBP2, LDHD, SLC2A3, GPI, ENO1, SLC25A11, PFKFB3, PFKM, LDHAL6B, SLC2A2, G6PC3, SLC2A6, GAPDHS, SLC2A11, PCK2, PFKP, PGK1, ALDOC, SLC2A10, ACYP2, SLC2A4, PKLR, HKDC1, PGK2, SLC2A8, PGAM1, SLC5A1, SLC5A12, SLC16A1, ALDOB, HK3, HK1, SLC5A9, GPD2, PFKFB1, SLC2A7, SLC5A11, SLC5A3, ACYP1, SLC16A8, PFKFB2, ALDOA, SLC5A2, HK2, ENO3, SLC2A12, FBP1, LDHA, LDHB, LDHC, G6PC, SLC2A14, SLC5A8, TPI1, SLC16A3, PKM2, ENO2, PGM1, UEVLD, LDHAL6A, SLC2A1, PGM2; Citric Acid Cycle group: ACLY, FAH, PC, MDH1B, SLC16A7, IREB2, PCK1, MDH1, SLC33A1, ALDH1B1, IDH3B, DLST, PDHB, MDH2, ACO1, IDH1, SLC5A6, HICDH, SLC16A8, GOT1, ME3, ME1, CS, OGDH, SDHA, ALDH5A1, CLYBL, SDHD, IDH3A, SLC25A1, ACSS2, SDHC, ACSS1, SUCLA2, SLC13A5, PDHX, SDHB, ALDH4A1, PCK2, DLD, ACO2, PDHA1, SLC13A2, FAHD1, IDH2, GOT2, ME2, ADSL, SUCLG2, SLC13A3, SUCLG1, SLC25A10, FH, IDH3G, SLC16A1, SLC25A11, PDHA2, DLAT; and, Fatty Acid Metabolism group: MLYCD, ALDH3A2, SLC27A5, SLC27A3, LIPC, SLC27A2, ACSL4, ACSL1, PCCB, SLC25A20, AADAC, SLC22A4, SLC22A5, ECH1, PCCA, SLC27A1, SLC27A4, CROT, ACSL5, ACSL3, CYP4F12.

In some embodiments, determining the gene group scores further comprises determining a respective gene group score for each of at least two of the following gene groups, using, for a particular gene group, RNA expression levels for at least three genes in the particular gene group to determine the gene group score for the particular group, the gene groups including: ECM associated group: ADAM8, ADAMTS4, C1QL3, CST7, CTSW, CXCL8, FASLG, LTB, MUC1, OSM, P4HA2, SCUBE1, SEMA4B, SEMA7A, SERPINE1, TCHH, TGFA, TGM2, TNFSF11, TNFSF9, WNT10B; TLS kidney group: ZNF683, POU2AF1, LAX1, CD79A, CXCL9, XCL2, JCHAIN, SLAMF7, CD38, SLAMF1, TNFRSF17, IRF4, HSH2D, PLA2G2D, MZB1; NRF2 signature group: TRIM16L, UGDH, KIAA1549, PANX2, FECH, LRP8, AKR1C2, FTH1, AKR1C3, CBR1, PFN2, CBX2, TXN, CYP4F11, CYP4F3, AKR1C1, AKR1B15, G6PD, PRDX1, TALDO1, EPT1, SRXN1, JAKMIP3, FTHL3, UCHL1, TXNRD1, Clorf131, CASKIN1, PGD, GPX2, OSGIN1, KIAA0319, CABYR, AIFM2, TRIM16, AKR1B10, GCLC, ABCC2, ETFB, IDH1, MAFG, NECAB2, ME1, PTGR1, PIR, GSR, RIT1, GCLM, ALDH3A1, NQO1, PKD1L2, NRG4, ABHD4, HRG, SLC7A11; and, tRCC signature group: FST, TRIM63, SLC10A2, ANTXRL, ERW-2, SNX22, INHBE, SV2B, FAM124A, EPHA5, LUZP2, CPEB1, HOXB13, ALLC, KCNF1, NDRG4, GREB1, ASTN1, JSRP1, UBE2U, KCNQ4, MYO7B, BRINP2, C1QL2, CCDC136, SLC51B, CATSPERG, PMEL, BIRC7, PLK5, ADARB2, CFAP61, TUBB4A, PLIN4, ABCB5, SYT3, HCN4, CTSK, SPACA1, TRIM67, NMRK2, LGI3, ARHGEF4, NTSR2, KEL, SNCB, PLD5, ADGRB1, CYP17A1, IGFBPL1, TRIM71, SLC45A2, TP73, IP6K3, HABP2, RGS20, IGFN1, CDH17.

In some embodiments, determining the gene group scores comprises determining a respective gene group score for each of the following gene groups, using, for each gene group, RNA expression levels for each of the genes in each gene group to determine the gene group score for each particular group, the gene groups including: Effector cells group: PRF1, GZMB, TBX21, CD8B, ZAP70, IFNG, GZMK, EOMES, FASLG, CD8A, GZMA, GNLY; NK cells group: GZMB, NKG7, CD160, GZMH, CD244, EOMES, KLRK1, NCR1, GNLY, KLRF1, FGFBP2, SH2D1B, KIR2DL4, IFNG, NCR3, KLRC2, CD226; T cells group: TRAC, TRBC2, TBX21, CD3E, CD3D, ITK, TRBC1, CD3G, CD28, TRAT1, CD5; B cells group: CR2, MS4A1, CD79A, FCRL5, STAP1, TNFRSF17, TNFRSF13B, CD19, BLK, CD79B, TNFRSF13C, CD22, PAX5; Antitumor cytokines group: IFNA2, CCL3, TNF, TNFSF10, IL21, IFNB1; Checkpoint inhibition group: CTLA4, HAVCR2, CD274, LAG3, BTLA, VSIR, PDCD1LG2, TIGIT, PDCD1; Treg group: TNFRSF18, IKZF2, IL10, IKZF4, CTLA4, FOXP3, CCR8; Neutrophil signature group: FCGR3B, CD177, CTSG, PGLYRP1, FFAR2, CXCR2, PRTN3, ELANE, MPO, CXCR1; Granulocyte traffic group: CXCL8, CCR3, CXCR2, CXCL2, CCL11, KITLG, CXCL1, CXCL5, CXCR1; MDSC group: ARG1, IL4I1, IL10, CYBB, IL6, PTGS2, IDO1; Macrophages group: MRC1, CD163, MSR1, SIGLEC1, IL4I1, CD68, IL10, CSF1R; Cancer-associated fibroblasts (CAF) group: PDGFRB, COL6A3, FBLN1, CXCL12, COL6A2, COL6A1, LUM, CD248, COL5A1, MMP2, COL1A1, MFAP5, PDGFRA, LRP1, FGF2, MMP3, FAP, COL1A2, ACTA2; Matrix group: COL11A1, LAMB3, FN1, COL1A1, COL4A1, ELN, LGALS9, LGALS7, LAMC2, TNC, LAMA3, COL3A1, COL5A1, VTN, COL1A2; Angiogenesis group: PGF, CXCL8, FLT1, ANGPT1, ANGPT2, VEGFC, VEGFB, CXCR2, VEGFA, VWF, CDH5, CXCL5, PDGFC, KDR, TEK; Endothelium group: NOS3, MMRN1, FLT1, CLEC14A, MMRN2, VCAM1, ENG, VWF, CDH5, KDR; Proliferation rate group: AURKA, MCM2, CCNB1, MYBL2, MCM6, CDK2, E2F1, CCNE1, ESCO2, CCND1, AURKB, BUB1, MKI67, PLK1, CETN3; EMT signature group: SNAI2, TWIST1, ZEB2, SNAI1, ZEB1, TWIST2, CDH2; Citric Acid Cycle group: ACLY, FAH, PC, MDH1B, SLC16A7, IREB2, PCK1, MDH1, SLC33A1, ALDH1B1, IDH3B, DLST, PDHB, MDH2, ACO1, IDH1, SLC5A6, HICDH, SLC16A8, GOT1, ME3, ME1, CS, OGDH, SDHA, ALDH5A1, CLYBL, SDHD, IDH3A, SLC25A1, ACSS2, SDHC, ACSS1, SUCLA2, SLC13A5, PDHX, SDHB, ALDH4A1, PCK2, DLD, ACO2, PDHA1, SLC13A2, FAHD1, IDH2, GOT2, ME2, ADSL, SUCLG2, SLC13A3, SUCLG1, SLC25A10, FH, IDH3G, SLC16A1, SLC25A11, PDHA2, DLAT; Glycolysis and Gluconeogenesis group: SLC2A9, PFKL, GCK, PFKFB4, SLC16A7, PCK1, PGAM2, GAPDH, BPGM, G6PC2, FBP2, LDHD, SLC2A3, GPI, ENO1, SLC25A11, PFKFB3, PFKM, LDHAL6B, SLC2A2, G6PC3, SLC2A6, GAPDHS, SLC2A11, PCK2, PFKP, PGK1, ALDOC, SLC2A10, ACYP2, SLC2A4, PKLR, HKDC1, PGK2, SLC2A8, PGAM1, SLC5A1, SLC5A12, SLC16A1, ALDOB, HK3, HK1, SLC5A9, GPD2, PFKFB1, SLC2A7, SLC5A11, SLC5A3, ACYP1, SLC16A8, PFKFB2, ALDOA, SLC5A2, HK2, ENO3, SLC2A12, FBP1, LDHA, LDHB, LDHC, G6PC, SLC2A14, SLC5A8, TPI1, SLC16A3, PKM2, ENO2, PGM1, UEVLD, LDHAL6A, SLC2A1, PGM2; and Fatty Acid Metabolism group: MLYCD, ALDH3A2, SLC27A5, SLC27A3, LIPC, SLC27A2, ACSL4, ACSL1, PCCB, SLC25A20, AADAC, SLC22A4, SLC22A5, ECH1, PCCA, SLC27A1, SLC27A4, CROT, ACSL5, ACSL3, CYP4F12.

In some embodiments, determining the gene group scores comprises determining a respective gene group score for each of the following gene groups, using, for each gene group, RNA expression levels for each of the genes in each gene group to determine the gene group score for each particular group, the gene groups including: MHC I group: HLA-C, B2M, HLA-B, HLA-A, TAP1, TAP2, NLRC5, TAPBP; MHC II group: HLA-DQA1, HLA-DMA, HLA-DRB1, HLA-DMB, CIITA, HLA-DPA1, HLA-DPB1, HLA-DRA, HLA-DQB1; Coactivation molecules group: CD80, TNFRSF4, CD27, CD83, TNFSF9, CD40LG, CD70, ICOS, CD86, CD40, TNFSF4, ICOSLG, TNFRSF9, CD28; Effector cells group: PRF1, GZMB, TBX21, CD8B, ZAP70, IFNG, GZMK, EOMES, FASLG, CD8A, GZMA, GNLY; T cell traffic group: CXCL9, CCL3, CXCR3, CXCL10, CXCL11, CCL5, CCL4, CX3CL1, CX3CR1; NK cells group: GZMB, NKG7, CD160, GZMH, CD244, EOMES, KLRK1, NCR1, GNLY, KLRF1, FGFBP2, SH2D1B, KIR2DL4, IFNG, NCR3, KLRC2, CD226; T cells group: TRAC, TRBC2, TBX21, CD3E, CD3D, ITK, TRBC1, CD3G, CD28, TRAT1, CD5; B cells group: CR2, MS4A1, CD79A, FCRL5, STAP1, TNFRSF17, TNFRSF13B, CD19, BLK, CD79B, TNFRSF13C, CD22, PAX5; M1 signatures group: IL1B, IL12B, NOS2, SOCS3, IRF5, IL23A, TNF, IL12A, CMKLR1; Th1 signature group: IL12RB2, IL2, TBX21, IFNG, STAT4, IL21, CD40LG; Antitumor cytokines group: IFNA2, CCL3, TNF, TNFSF10, IL21, IFNB1; Checkpoint inhibition group: CTLA4, HAVCR2, CD274, LAG3, BTLA, VSIR, PDCD1LG2, TIGIT, PDCD1; Treg group: TNFRSF18, IKZF2, IL10, IKZF4, CTLA4, FOXP3, CCR8; T reg traffic group: CCL28, CCR10, CCR4, CCR8, CCL17, CCL22, CCL1; Neutrophil signature group: FCGR3B, CD177, CTSG, PGLYRP1, FFAR2, CXCR2, PRTN3, ELANE, MPO, CXCR1; Granulocyte traffic group: CXCL8, CCR3, CXCR2, CXCL2, CCL11, KITLG, CXCL1, CXCL5, CXCR1; MDSC group: ARG1, IL4I1, IL10, CYBB, IL6, PTGS2, IDO1; MDSC traffic group: CCL15, IL6R, CSF2RA, CSF2, CXCL8, CXCL12, IL6, CSF3, CCL26, CXCR4, CXCR2, CSF3R, CSF1, CXCL5, CSF1R; Macrophages group: MRC1, CD163, MSR1, SIGLEC1, IL4I1, CD68, IL10, CSF1R; Macrophage DC traffic group: CCL7, CCL2, XCR1, XCL1, CSF1, CCR2, CCL8, CSF1R; Th2 signature group: IL13, CCR4, IL10, IL5, IL4; Protumor cytokines group: MIF, TGFB1, IL10, TGFB3, IL6, TGFB2, IL22; CAF group: PDGFRB, COL6A3, FBLN1, CXCL12, COL6A2, COL6A1, LUM, CD248, COL5A1, MMP2, COL1A1, MFAP5, PDGFRA, LRP1, FGF2, MMP3, FAP, COL1A2, ACTA2; Matrix group: COL11A1, LAMB3, FN1, COL1A1, COL4A1, ELN, LGALS9, LGALS7, LAMC2, TNC, LAMA3, COL3A1, COL5A1, VTN, COL1A2; Matrix remodeling group: MMP1, PLOD2, MMP2, MMP12, ADAMTS5, ADAMTS4, LOX, MMP9, MMP11, MMP3, MMP1, CA9; Angiogenesis group: PGF, CXCL8, FLT1, ANGPT1, ANGPT2, VEGFC, VEGFB, CXCR2, VEGFA, VWF, CDH5, CXCL5, PDGFC, KDR, TEK; Endothelium group: NOS3, MMRN1, FLT1, CLEC14A, MMRN2, VCAM1, ENG, VWF, CDH5, KDR; Proliferation rate group: AURKA, MCM2, CCNB1, MYBL2, MCM6, CDK2, E2F1, CCNE1, ESCO2, CCND1, AURKB, BUB1, MKI67, PLK1, CETN3; EMT signature group: SNAI2, TWIST1, ZEB2, SNAI1, ZEB1, TWIST2, CDH2; Cyclic Nucleotides Metabolism group: ADCY4, PDE11A, PDE6A, PDE9A, PDE6C, ADCY7, PDE4A, PDE8A, PDE1B, PDE1A, GUCY2C, GUCY1A3, ADCY9, ADCY2, PDE6B, ADCY8, PDE8B, GUCY2F, PDE4C, PDE3A, GUCY1A2, PDE6G, PDE1C, GUCY2D, ADCY10, GUCY1B3, GUCY1B2, PDE7B, PDE5A, PDE6D, NPR2, ADCY5, NPR1, ADCY6, PDE7A, PDE2A, PDE4B, PDE10A, PDE6H, PDE4D, ADCY1, PDE3B, ADCY3; Glycolysis and Gluconeogenesis group: SLC2A9, PFKL, GCK, PFKFB4, SLC16A7, PCK1, PGAM2, GAPDH, BPGM, G6PC2, FBP2, LDHD, SLC2A3, GPI, ENO1, SLC25A11, PFKFB3, PFKM, LDHAL6B, SLC2A2, G6PC3, SLC2A6, GAPDHS, SLC2A11, PCK2, PFKP, PGK1, ALDOC, SLC2A10, ACYP2, SLC2A4, PKLR, HKDC1, PGK2, SLC2A8, PGAM1, SLC5A1, SLC5A12, SLC16A1, ALDOB, HK3, HK1, SLC5A9, GPD2, PFKFB1, SLC2A7, SLC5A11, SLC5A3, ACYP1, SLC16A8, PFKFB2, ALDOA, SLC5A2, HK2, ENO3, SLC2A12, FBP1, LDHA, LDHB, LDHC, G6PC, SLC2A14, SLC5A8, TPI1, SLC16A3, PKM2, ENO2, PGM1, UEVLD, LDHAL6A, SLC2A1, PGM2; Citric Acid Cycle group: ACLY, FAH, PC, MDH1B, SLC16A7, IREB2, PCK1, MDH1, SLC33A1, ALDH1B1, IDH3B, DLST, PDHB, MDH2, ACO1, IDH1, SLC5A6, HICDH, SLC16A8, GOT1, ME3, ME1, CS, OGDH, SDHA, ALDH5A1, CLYBL, SDHD, IDH3A, SLC25A1, ACSS2, SDHC, ACSS1, SUCLA2, SLC13A5, PDHX, SDHB, ALDH4A1, PCK2, DLD, ACO2, PDHA1, SLC13A2, FAHD1, IDH2, GOT2, ME2, ADSL, SUCLG2, SLC13A3, SUCLG1, SLC25A10, FH, IDH3G, SLC16A1, SLC25A11, PDHA2, DLAT; and, Fatty Acid Metabolism group: MLYCD, ALDH3A2, SLC27A5, SLC27A3, LIPC, SLC27A2, ACSL4, ACSL1, PCCB, SLC25A20, AADAC, SLC22A4, SLC22A5, ECH1, PCCA, SLC27A1, SLC27A4, CROT, ACSL5, ACSL3, CYP4F12.

In some embodiments, determining the gene group scores further comprises: determining a respective gene group score for each of the following gene groups, using, for each gene group, RNA expression levels for each of the genes in each gene group to determine the gene group score for each particular group, the gene groups including: ECM associated group: ADAMS, ADAMTS4, C1QL3, CST7, CTSW, CXCL8, FASLG, LTB, MUC1, OSM, P4HA2, SCUBE1, SEMA4B, SEMA7A, SERPINE1, TCHH, TGFA, TGM2, TNFSF11, TNFSF9, WNT10B; TLS kidney group: ZNF683, POU2AF1, LAX1, CD79A, CXCL9, XCL2, JCHAIN, SLAMF7, CD38, SLAMF1, TNFRSF17, IRF4, HSH2D, PLA2G2D, MZB1; NRF2 signature group: TRIM16L, UGDH, KIAA1549, PANX2, FECH, LRP8, AKR1C2, FTH1, AKR1C3, CBR1, PFN2, CBX2, TXN, CYP4F11, CYP4F3, AKR1C1, AKR1B15, G6PD, PRDX1, TALDO1, EPT1, SRXN1, JAKMIP3, FTHL3, UCHL1, TXNRD1, Clorf131, CASKIN1, PGD, GPX2, OSGIN1, KIAA0319, CABYR, AIFM2, TRIM16, AKR1B10, GCLC, ABCC2, ETFB, IDH1, MAFG, NECAB2, ME1, PTGR1, PIR, GSR, RIT1, GCLM, ALDH3A1, NQO1, PKD1L2, NRG4, ABHD4, HRG, SLC7A11; and, tRCC signature group: FST, TRIM63, SLC10A2, ANTXRL, ERVV-2, SNX22, INHBE, SV2B, FAM124A, EPHA5, LUZP2, CPEB1, HOXB13, ALLC, KCNF1, NDRG4, GREB1, ASTN1, JSRP1, UBE2U, KCNQ4, MYO7B, BRINP2, C1QL2, CCDC136, SLC51B, CATSPERG, PMEL, BIRC7, PLK5, ADARB2, CFAP61, TUBB4A, PLIN4, ABCB5, SYT3, HCN4, CTSK, SPACA1, TRIM67, NMRK2, LGI3, ARHGEF4, NTSR2, KEL, SNCB, PLD5, ADGRB1, CYP17A1, IGFBPL1, TRIM71, SLC45A2, TP73, IP6K3, HABP2, RGS20, IGFN1, CDH17.

In some embodiments, determining the gene group scores comprises determining a first score of a first gene group using a single-sample GSEA (ssGSEA) technique from RNA expression levels for at least some of the genes in one of the following gene groups: MHC I group: HLA-C, B2M, HLA-B, HLA-A, TAP1, TAP2, NLRC5, TAPBP; MHC II group: HLA-DQA1, HLA-DMA, HLA-DRB1, HLA-DMB, CIITA, HLA-DPA1, HLA-DPB1, HLA-DRA, HLA-DQB1; Coactivation molecules group: CD80, TNFRSF4, CD27, CD83, TNFSF9, CD40LG, CD70, ICOS, CD86, CD40, TNFSF4, ICOSLG, TNFRSF9, CD28; Effector cells group: PRF1, GZMB, TBX21, CD8B, ZAP70, IFNG, GZMK, EOMES, FASLG, CD8A, GZMA, GNLY; T cell traffic group: CXCL9, CCL3, CXCR3, CXCL10, CXCL11, CCL5, CCL4, CX3CL1, CX3CR1; NK cells group: GZMB, NKG7, CD160, GZMH, CD244, EOMES, KLRK1, NCR1, GNLY, KLRF1, FGFBP2, SH2D1B, KIR2DL4, IFNG, NCR3, KLRC2, CD226; T cells group: TRAC, TRBC2, TBX21, CD3E, CD3D, ITK, TRBC1, CD3G, CD28, TRAT1, CD5; B cells group: CR2, MS4A1, CD79A, FCRL5, STAP1, TNFRSF17, TNFRSF13B, CD19, BLK, CD79B, TNFRSF13C, CD22, PAX5; M1 signatures group: IL1B, IL12B, NOS2, SOCS3, IRF5, IL23A, TNF, IL12A, CMKLR1; Th1 signature group: IL12RB2, IL2, TBX21, IFNG, STAT4, IL21, CD40LG; Antitumor cytokines group: IFNA2, CCL3, TNF, TNFSF10, IL21, IFNB1; Checkpoint inhibition group: CTLA4, HAVCR2, CD274, LAG3, BTLA, VSIR, PDCD1LG2, TIGIT, PDCD1; Treg group: TNFRSF18, IKZF2, IL10, IKZF4, CTLA4, FOXP3, CCR8; T reg traffic group: CCL28, CCR10, CCR4, CCR8, CCL17, CCL22, CCL1; Neutrophil signature group: FCGR3B, CD177, CTSG, PGLYRP1, FFAR2, CXCR2, PRTN3, ELANE, MPO, CXCR1; Granulocyte traffic group: CXCL8, CCR3, CXCR2, CXCL2, CCL11, KITLG, CXCL1, CXCL5, CXCR1; MDSC group: ARG1, IL4I1, IL10, CYBB, IL6, PTGS2, IDO1; MDSC traffic group: CCL15, IL6R, CSF2RA, CSF2, CXCL8, CXCL12, IL6, CSF3, CCL26, CXCR4, CXCR2, CSF3R, CSF1, CXCL5, CSF1R; Macrophages group: MRC1, CD163, MSR1, SIGLEC1, IL4I1, CD68, IL10, CSF1R; Macrophage DC traffic group: CCL7, CCL2, XCR1, XCL1, CSF1, CCR2, CCL8, CSF1R; Th2 signature group: IL13, CCR4, IL10, IL5, IL4; Protumor cytokines group: MIF, TGFB1, IL10, TGFB3, IL6, TGFB2, IL22; CAF group: PDGFRB, COL6A3, FBLN1, CXCL12, COL6A2, COL6A1, LUM, CD248, COL5A1, MMP2, COL1A1, MFAP5, PDGFRA, LRP1, FGF2, MMP3, FAP, COL1A2, ACTA2; Matrix group: COL11A1, LAMB3, FN1, COL1A1, COL4A1, ELN, LGALS9, LGALS7, LAMC2, TNC, LAMA3, COL3A1, COL5A1, VTN, COL1A2; Matrix remodeling group: MMP1, PLOD2, MMP2, MMP12, ADAMTS5, ADAMTS4, LOX, MMP9, MMP11, MMP3, MMP1, CA9; Angiogenesis group: PGF, CXCL8, FLT1, ANGPT1, ANGPT2, VEGFC, VEGFB, CXCR2, VEGFA, VWF, CDH5, CXCL5, PDGFC, KDR, TEK; Endothelium group: NOS3, MMRN1, FLT1, CLEC14A, MMRN2, VCAM1, ENG, VWF, CDH5, KDR; Proliferation rate group: AURKA, MCM2, CCNB1, MYBL2, MCM6, CDK2, E2F1, CCNE1, ESCO2, CCND1, AURKB, BUB1, MKI67, PLK1, CETN3; EMT signature group: SNAI2, TWIST1, ZEB2, SNAI1, ZEB1, TWIST2, CDH2; Cyclic Nucleotides Metabolism group: ADCY4, PDE11A, PDE6A, PDE9A, PDE6C, ADCY7, PDE4A, PDE8A, PDE1B, PDE1A, GUCY2C, GUCY1A3, ADCY9, ADCY2, PDE6B, ADCY8, PDE8B, GUCY2F, PDE4C, PDE3A, GUCY1A2, PDE6G, PDE1C, GUCY2D, ADCY10, GUCY1B3, GUCY1B2, PDE7B, PDE5A, PDE6D, NPR2, ADCY5, NPR1, ADCY6, PDE7A, PDE2A, PDE4B, PDE10A, PDE6H, PDE4D, ADCY1, PDE3B, ADCY3; Glycolysis and Gluconeogenesis group: SLC2A9, PFKL, GCK, PFKFB4, SLC16A7, PCK1, PGAM2, GAPDH, BPGM, G6PC2, FBP2, LDHD, SLC2A3, GPI, ENO1, SLC25A11, PFKFB3, PFKM, LDHAL6B, SLC2A2, G6PC3, SLC2A6, GAPDHS, SLC2A11, PCK2, PFKP, PGK1, ALDOC, SLC2A10, ACYP2, SLC2A4, PKLR, HKDC1, PGK2, SLC2A8, PGAM1, SLC5A1, SLC5A12, SLC16A1, ALDOB, HK3, HK1, SLC5A9, GPD2, PFKFB1, SLC2A7, SLC5A11, SLC5A3, ACYP1, SLC16A8, PFKFB2, ALDOA, SLC5A2, HK2, ENO3, SLC2A12, FBP1, LDHA, LDHB, LDHC, G6PC, SLC2A14, SLC5A8, TPI1, SLC16A3, PKM2, ENO2, PGM1, UEVLD, LDHAL6A, SLC2A1, PGM2; Citric Acid Cycle group: ACLY, FAH, PC, MDH1B, SLC16A7, IREB2, PCK1, MDH1, SLC33A1, ALDH1B1, IDH3B, DLST, PDHB, MDH2, ACO1, IDH1, SLC5A6, HICDH, SLC16A8, GOT1, ME3, ME1, CS, OGDH, SDHA, ALDH5A1, CLYBL, SDHD, IDH3A, SLC25A1, ACSS2, SDHC, ACSS1, SUCLA2, SLC13A5, PDHX, SDHB, ALDH4A1, PCK2, DLD, ACO2, PDHA1, SLC13A2, FAHD1, IDH2, GOT2, ME2, ADSL, SUCLG2, SLC13A3, SUCLG1, SLC25A10, FH, IDH3G, SLC16A1, SLC25A11, PDHA2, DLAT; and, Fatty Acid Metabolism group: MLYCD, ALDH3A2, SLC27A5, SLC27A3, LIPC, SLC27A2, ACSL4, ACSL1, PCCB, SLC25A20, AADAC, SLC22A4, SLC22A5, ECH1, PCCA, SLC27A1, SLC27A4, CROT, ACSL5, ACSL3, CYP4F12.

In some embodiments, determining the gene group scores comprises using a single-sample GSEA (ssGSEA) technique to determine the gene group scores from RNA expression levels for each of the genes in each of the following gene groups: MHC I group: HLA-C, B2M, HLA-B, HLA-A, TAP1, TAP2, NLRC5, TAPBP; MHC II group: HLA-DQA1, HLA-DMA, HLA-DRB1, HLA-DMB, CIITA, HLA-DPA1, HLA-DPB1, HLA-DRA, HLA-DQB1; Coactivation molecules group: CD80, TNFRSF4, CD27, CD83, TNFSF9, CD40LG, CD70, ICOS, CD86, CD40, TNFSF4, ICOSLG, TNFRSF9, CD28; Effector cells group: PRF1, GZMB, TBX21, CD8B, ZAP70, IFNG, GZMK, EOMES, FASLG, CD8A, GZMA, GNLY; T cell traffic group: CXCL9, CCL3, CXCR3, CXCL10, CXCL11, CCL5, CCL4, CX3CL1, CX3CR1; NK cells group: GZMB, NKG7, CD160, GZMH, CD244, EOMES, KLRK1, NCR1, GNLY, KLRF1, FGFBP2, SH2D1B, KIR2DL4, IFNG, NCR3, KLRC2, CD226; T cells group: TRAC, TRBC2, TBX21, CD3E, CD3D, ITK, TRBC1, CD3G, CD28, TRAT1, CD5; B cells group: CR2, MS4A1, CD79A, FCRL5, STAP1, TNFRSF17, TNFRSF13B, CD19, BLK, CD79B, TNFRSF13C, CD22, PAX5; M1 signatures group: IL1B, IL12B, NOS2, SOCS3, IRF5, IL23A, TNF, IL12A, CMKLR1; Th1 signature group: IL12RB2, IL2, TBX21, IFNG, STAT4, IL21, CD40LG; Antitumor cytokines group: IFNA2, CCL3, TNF, TNFSF10, IL21, IFNB1; Checkpoint inhibition group: CTLA4, HAVCR2, CD274, LAG3, BTLA, VSIR, PDCD1LG2, TIGIT, PDCD1; Treg group: TNFRSF18, IKZF2, IL10, IKZF4, CTLA4, FOXP3, CCR8; T reg traffic group: CCL28, CCR10, CCR4, CCR8, CCL17, CCL22, CCL1; Neutrophil signature group: FCGR3B, CD177, CTSG, PGLYRP1, FFAR2, CXCR2, PRTN3, ELANE, MPO, CXCR1; Granulocyte traffic group: CXCL8, CCR3, CXCR2, CXCL2, CCL11, KITLG, CXCL1, CXCL5, CXCR1; MDSC group: ARG1, IL4I1, IL10, CYBB, IL6, PTGS2, IDO1; MDSC traffic group: CCL15, IL6R, CSF2RA, CSF2, CXCL8, CXCL12, IL6, CSF3, CCL26, CXCR4, CXCR2, CSF3R, CSF1, CXCL5, CSF1R; Macrophages group: MRC1, CD163, MSR1, SIGLEC1, IL4I1, CD68, IL10, CSF1R; Macrophage DC traffic group: CCL7, CCL2, XCR1, XCL1, CSF1, CCR2, CCL8, CSF1R; Th2 signature group: IL13, CCR4, IL10, IL5, IL4; Protumor cytokines group: MIF, TGFB1, IL10, TGFB3, IL6, TGFB2, IL22; CAF group: PDGFRB, COL6A3, FBLN1, CXCL12, COL6A2, COL6A1, LUM, CD248, COL5A1, MMP2, COL1A1, MFAP5, PDGFRA, LRP1, FGF2, MMP3, FAP, COL1A2, ACTA2; Matrix group: COL11A1, LAMB3, FN1, COL1A1, COL4A1, ELN, LGALS9, LGALS7, LAMC2, TNC, LAMA3, COL3A1, COL5A1, VTN, COL1A2; Matrix remodeling group: MMP1, PLOD2, MMP2, MMP12, ADAMTS5, ADAMTS4, LOX, MMP9, MMP11, MMP3, MMP1, CA9; Angiogenesis group: PGF, CXCL8, FLT1, ANGPT1, ANGPT2, VEGFC, VEGFB, CXCR2, VEGFA, VWF, CDH5, CXCL5, PDGFC, KDR, TEK; Endothelium group: NOS3, MMRN1, FLT1, CLEC14A, MMRN2, VCAM1, ENG, VWF, CDH5, KDR; Proliferation rate group: AURKA, MCM2, CCNB1, MYBL2, MCM6, CDK2, E2F1, CCNE1, ESCO2, CCND1, AURKB, BUB1, MKI67, PLK1, CETN3; EMT signature group: SNAI2, TWIST1, ZEB2, SNAI1, ZEB1, TWIST2, CDH2; Cyclic Nucleotides Metabolism group: ADCY4, PDE11A, PDE6A, PDE9A, PDE6C, ADCY7, PDE4A, PDE8A, PDE1B, PDE1A, GUCY2C, GUCY1A3, ADCY9, ADCY2, PDE6B, ADCY8, PDE8B, GUCY2F, PDE4C, PDE3A, GUCY1A2, PDE6G, PDE1C, GUCY2D, ADCY10, GUCY1B3, GUCY1B2, PDE7B, PDE5A, PDE6D, NPR2, ADCY5, NPR1, ADCY6, PDE7A, PDE2A, PDE4B, PDE10A, PDE6H, PDE4D, ADCY1, PDE3B, ADCY3; Glycolysis and Gluconeogenesis group: SLC2A9, PFKL, GCK, PFKFB4, SLC16A7, PCK1, PGAM2, GAPDH, BPGM, G6PC2, FBP2, LDHD, SLC2A3, GPI, ENO1, SLC25A11, PFKFB3, PFKM, LDHAL6B, SLC2A2, G6PC3, SLC2A6, GAPDHS, SLC2A11, PCK2, PFKP, PGK1, ALDOC, SLC2A10, ACYP2, SLC2A4, PKLR, HKDC1, PGK2, SLC2A8, PGAM1, SLC5A1, SLC5A12, SLC16A1, ALDOB, HK3, HK1, SLC5A9, GPD2, PFKFB1, SLC2A7, SLC5A11, SLC5A3, ACYP1, SLC16A8, PFKFB2, ALDOA, SLC5A2, HK2, ENO3, SLC2A12, FBP1, LDHA, LDHB, LDHC, G6PC, SLC2A14, SLC5A8, TPI1, SLC16A3, PKM2, ENO2, PGM1, UEVLD, LDHAL6A, SLC2A1, PGM2; Citric Acid Cycle group: ACLY, FAH, PC, MDH1B, SLC16A7, IREB2, PCK1, MDH1, SLC33A1, ALDH1B1, IDH3B, DLST, PDHB, MDH2, ACO1, IDH1, SLC5A6, HICDH, SLC16A8, GOT1, ME3, ME1, CS, OGDH, SDHA, ALDH5A1, CLYBL, SDHD, IDH3A, SLC25A1, ACSS2, SDHC, ACSS1, SUCLA2, SLC13A5, PDHX, SDHB, ALDH4A1, PCK2, DLD, ACO2, PDHA1, SLC13A2, FAHD1, IDH2, GOT2, ME2, ADSL, SUCLG2, SLC13A3, SUCLG1, SLC25A10, FH, IDH3G, SLC16A1, SLC25A11, PDHA2, DLAT; and, Fatty Acid Metabolism group: MLYCD, ALDH3A2, SLC27A5, SLC27A3, LIPC, SLC27A2, ACSL4, ACSL1, PCCB, SLC25A20, AADAC, SLC22A4, SLC22A5, ECH1, PCCA, SLC27A1, SLC27A4, CROT, ACSL5, ACSL3, CYP4F12.

In some embodiments, determining the gene group scores is performed using a single-sample GSEA (ssGSEA) technique and using RNA expression levels for each of the genes in each of the following gene groups: ECM associated group: ADAM8, ADAMTS4, C1QL3, CST7, CTSW, CXCL8, FASLG, LTB, MUC1, OSM, P4HA2, SCUBE1, SEMA4B, SEMA7A, SERPINE1, TCHH, TGFA, TGM2, TNFSF11, TNFSF9, WNT10B; TLS kidney group: ZNF683, POU2AF1, LAX1, CD79A, CXCL9, XCL2, JCHAIN, SLAMF7, CD38, SLAMF1, TNFRSF17, IRF4, HSH2D, PLA2G2D, MZB1; NRF2 signature group: TRIM16L, UGDH, KIAA1549, PANX2, FECH, LRP8, AKR1C2, FTH1, AKR1C3, CBR1, PFN2, CBX2, TXN, CYP4F11, CYP4F3, AKR1C1, AKR1B15, G6PD, PRDX1, TALDO1, EPT1, SRXN1, JAKMIP3, FTHL3, UCHL1, TXNRD1, Clorf131, CASKIN1, PGD, GPX2, OSGIN1, KIAA0319, CABYR, AIFM2, TRIM16, AKR1B10, GCLC, ABCC2, ETFB, IDH1, MAFG, NECAB2, ME1, PTGR1, PIR, GSR, RIT1, GCLM, ALDH3A1, NQO1, PKD1L2, NRG4, ABHD4, HRG, SLC7A11; and, tRCC signature group: FST, TRIM63, SLC10A2, ANTXRL, ERVV-2, SNX22, INHBE, SV2B, FAM124A, EPHA5, LUZP2, CPEB1, HOXB13, ALLC, KCNF1, NDRG4, GREB1, ASTN1, JSRP1, UBE2U, KCNQ4, MYO7B, BRINP2, C1QL2, CCDC136, SLC51B, CATSPERG, PMEL, BIRC7, PLK5, ADARB2, CFAP61, TUBB4A, PLIN4, ABCB5, SYT3, HCN4, CTSK, SPACA1, TRIM67, NMRK2, LGI3, ARHGEF4, NTSR2, KEL, SNCB, PLD5, ADGRB1, CYP17A1, IGFBPL1, TRIM71, SLC45A2, TP73, IP6K3, HABP2, RGS20, IGFN1, CDH17.

In some embodiments, generating the RC TME signature further comprises normalizing the gene group scores. In some embodiments, the normalizing comprises applying median scaling to the gene group scores.

In some embodiments, the plurality of RC TME types is associated with a respective plurality of RC TME signature clusters, wherein identifying, using the RC TME signature and from among a plurality of RC TME types, the RC TME type for the subject comprises associating the RC TME signature of the subject with a particular one of the plurality of RC TME signature clusters; and identifying the RC TME type for the subject as the RC TME type corresponding to the particular one of the plurality of RC TME signature clusters to which the RC TME signature of the subject is associated.

In some embodiments, methods described herein further comprise generating the plurality of RC TME signature clusters, the generating comprising obtaining multiple sets of RNA expression data by sequencing biological samples from multiple respective subjects, each of the multiple sets of RNA expression data indicating RNA expression levels for at least some genes in each of the at least some of the plurality of gene groups listed in Table 1; generating multiple RC TME signatures from the multiple sets of RNA expression data, each of the multiple RC TME signatures comprising gene group expression scores for respective gene groups in the plurality of gene groups, the generating comprising, for each particular one of the multiple RC TME signatures: determining the RC TME signature by determining the gene group expression scores using the RNA expression levels in the particular set of RNA expression data for which the particular one RC TME signature is being generated; and clustering the multiple RC signatures to obtain the plurality of RC TME signature clusters.

In some embodiments, the clustering comprises dense clustering, spectral clustering, k-means clustering, hierarchical clustering, and/or an agglomerative clustering.

In some embodiments, methods further comprise updating the plurality of RC TME signature clusters using the RC TME signature of the subject, wherein the RC TME signature of the subject is one of a threshold number RC TME signatures for a threshold number of subjects, wherein when the threshold number of RC TME signatures is generated the RC TME signature clusters are updated, wherein the threshold number of RC TME signatures is at least 50, at least 75, at least 100, at least 200, at least 500, at least 1000, or at least 5000 RC TME signatures.

In some embodiments, the updating is performed using a clustering algorithm selected from the group consisting of a dense clustering algorithm, spectral clustering algorithm, k-means clustering algorithm, hierarchical clustering algorithm, and an agglomerative clustering algorithm.

In some embodiments, the methods further comprise determining an RC TME type of a second subject, wherein the RC TME type of the second subject is identified using the updated RC TME signature clusters, wherein the identifying comprises determining an RC TME signature of the second subject from RNA expression data obtained by sequencing a biological sample obtained from the second subject; associating the RC TME signature of the second subject with a particular one of the plurality of the updated RC TME signature clusters; and identifying the RC TME type for the second subject as the RC TME type corresponding to the particular one of the plurality of updated RC TME signature clusters to which the RC TME signature of the second subject is associated.

In some embodiments, the plurality of RC TME types comprises: RC TME type A, RC TME type B, RC TME type C, RC TME type D, and RC TME type E.

In some embodiments, the methods further comprise identifying at least one therapeutic agent for administration to the subject using the RC TME type of the subject. In some embodiments, the at least one therapeutic agent comprises an immuno-oncology (IO) agent. In some embodiments, the at least one therapeutic agent comprises a tyrosine kinase inhibitor (TKI).

In some embodiments, identifying the at least one therapeutic agent based upon the RC TME type of the subject comprises identifying a TKI as the at least one therapeutic agent when the subject is identified as having RC TME type E.

In some embodiments, identifying the at least one therapeutic agent based upon the RC TME type of the subject comprises identifying a combination of a TKI and an IO agent as the at least one therapeutic agent when the subject is identified as having RC TME type A or RC TME type B.

In some embodiments, the methods further comprise administering the at least one identified therapeutic agent to the subject.

In some embodiments, the methods comprise normalizing the RNA expression data to transcripts per million (TPM) units prior to generating an RC myogenesis signature.

In some embodiments, RNA expression levels comprise RNA expression levels for at least three of the following genes: CASQ1, TNNI1, MB, MYLPF, MYH7, CKM, MYL2, MYL1, CSRP3, ACTA1, MYOZ1, TNNT3, TNNC2, and TNNC1.

In some embodiments, RNA expression levels comprise RNA expression levels for each of the following genes: CASQ1, TNNI1, MB, MYLPF, MYH7, CKM, MYL2, MYL1, CSRP3, ACTA1, MYOZ1, TNNT3, TNNC2, and TNNC1.

In some embodiments, an RC myogenesis signature is determined, using a single-sample GSEA (ssGSEA) technique, from RNA expression levels for each of the following genes: CASQ1, TNNI1, MB, MYLPF, MYH7, CKM, MYL2, MYL1, CSRP3, ACTA1, MYOZ1, TNNT3, TNNC2, and TNNC1.

In some embodiments, methods described by the disclosure further comprise determining whether the value of an RC myogenesis signature is greater than a specified threshold. In some embodiments, the specified threshold is 4.

In some embodiments, when the value of an RC myogenesis signature is greater than the specified threshold, the method further comprises identifying the subject as a non-responder to an immuno-oncology (IO) agent.

In some embodiments, the methods further comprise identifying one or more non-IO agents for the subject. In some embodiments, the one or more non-immunotherapeutic agents comprises a TKI. In some embodiments, the methods further comprise administering the identified one or more non-IO agents to the subject.

In some embodiments, generating a set of input features comprises determining the RC TME type for the subject, using RNA expression data as described herein.

In some embodiments, generating the set of input features comprises determining the RNA expression levels for one or more of the following genes: PD1, PD-L1, and PD-L2.

In some embodiments, generating the set of input features comprises determining the ECM associated signature for the subject using the RNA expression data by performing ssGSEA on the RNA expression data for at least three of the “ECM associated signature” genes listed in Table 1. In some embodiments, determining the ECM associated signature further comprises performing ssGSEA on the RNA expression data for at least 4, 5, 6, 7, 8, 9, or 10 of the “ECM associated signature” genes listed in Table 1. In some embodiments, determining the ECM associated signature further comprises performing ssGSEA on the RNA expression data for each of the “ECM associated signature” genes listed in Table 1.

In some embodiments, generating the set of input features comprises determining the Angiogenesis signature for the subject using the RNA expression data by performing ssGSEA on the RNA expression data for at least three of the “Angiogenesis” genes listed in Table 1. In some embodiments, determining the Angiogenesis signature further comprises performing ssGSEA on the RNA expression data for at least 4, 5, 6, 7, 8, 9, or 10 of the “Angiogenesis” genes listed in Table 1. In some embodiments, determining the Angiogenesis signature further comprises performing ssGSEA on the RNA expression data for each of the “Angiogenesis” genes listed in Table 1.

In some embodiments, generating the set of input features comprises determining the Proliferation rate signature for the subject using the RNA expression data by performing ssGSEA on the RNA expression data for at least three of the “Proliferation rate” genes listed in Table 1. In some embodiments, determining the Proliferation rate signature further comprises performing ssGSEA on the RNA expression data for at least 4, 5, 6, 7, 8, 9, or 10 of the “Proliferation rate” genes listed in Table 1. In some embodiments, determining the Proliferation rate signature further comprises performing ssGSEA on the RNA expression data for each of the “Proliferation rate” genes listed in Table 1.

In some embodiments, generating the set of input features comprises determining the similarity score by comparing the gene group scores of an RC TME signature of the subject to an average of gene group scores of a plurality of RC TME signatures from RC TME type B samples and/or an average of gene group scores of a plurality of RC TME signatures from RC TME type C samples.

In some embodiments, determining the similarity score comprises calculating a Spearman correlation coefficient between the gene group scores for the respective plurality of gene groups of an RC TME signature of the subject; and averaged gene group scores for a plurality of gene groups of other RC type B and/or RC type C samples.

In some embodiments, the methods further comprise identifying the subject as being “IO-low” when the responder score is ≤0.05; “IO-medium” when the responder score is ≥0.05 and <0.5; or (iii) “IO-high” when the responder score is ≥0.5.

In some embodiments, a specified threshold is 0.5.

In some embodiments, methods described herein further comprise identifying an IO agent for administration to the subject when the responder score of the subject is above the specified threshold or wherein the subject is identified as being “IO-high”.

In some embodiments, the methods further comprise administering an IO agent to the subject when the responder score of the subject is above the specified threshold or wherein the subject is identified as being “IO-high”. In some embodiments, the IO agent comprises a PD1 inhibitor, a PD-L1 inhibitor, a PD-L2 inhibitor, or a CTLA-4 inhibitor.

In some embodiments, RNA expression data comprises the mean of scaled expression levels of PD1 and PDL1.

In some embodiments, methods described by the disclosure further comprise determining whether the subject comprises one or more of the following biomarkers Ploidy >4; a value of a RC myogenesis signature for the subject is greater than 4; one or more mTOR activating mutations; and/or one or more mutations in a gene or genes associated with antigen presentation. In some embodiments, the determining takes place prior to the input features being input into a machine learning model.

In some embodiments, methods described by the disclosure further comprise identifying the subject as having a responder score of 0 when the subject comprises one or more of the biomarkers.

In some embodiments, generating a set of input features comprises determining an RC TME type for the subject, using the RNA expression data as described herein.

In some embodiments, generating a set of input features comprises determining the Macrophage signature for the subject using the RNA expression data by performing ssGSEA on the RNA expression data for at least three of the “Macrophages” genes listed in Table 1. In some embodiments, determining the Macrophage signature further comprises performing ssGSEA on the RNA expression data for at least 4, 5, 6, 7, 8, 9, or 10 of the “Macrophages” genes listed in Table 1. In some embodiments, determining the Macrophage signature further comprises performing ssGSEA on the RNA expression data for each of the “Macrophages” genes listed in Table 1.

In some embodiments, generating the a of input features comprises determining the Angiogenesis signature for the subject using the RNA expression data by performing ssGSEA on the RNA expression data for at least three of the “Angiogenesis” genes listed in Table 1. In some embodiments, determining the Angiogenesis signature further comprises performing ssGSEA on the RNA expression data for at least 4, 5, 6, 7, 8, 9, or 10 of the “Angiogenesis” genes listed in Table 1. In some embodiments, determining the Angiogenesis signature further comprises performing ssGSEA on the RNA expression data for each of the “Angiogenesis” genes listed in Table 1.

In some embodiments, generating a set of input features comprises determining the Proliferation rate signature for the subject using the RNA expression data by performing ssGSEA on the RNA expression data for at least three of the “Proliferation rate” genes listed in Table 1. In some embodiments, determining the Proliferation rate signature further comprises performing ssGSEA on the RNA expression data for at least 4, 5, 6, 7, 8, 9, or 10 of the “Proliferation rate” genes listed in Table 1. In some embodiments, determining the Proliferation rate signature further comprises performing ssGSEA on the RNA expression data for each of the “Proliferation rate” genes listed in Table 1.

In some embodiments, generating a set of input features comprises determining the similarity score by comparing the gene group scores of an RC TME signature of the subject to an average of gene group scores of a plurality of RC TME signatures from RC TME type B samples. In some embodiments, determining the similarity score comprises calculating a Spearman correlation coefficient between: the gene group scores for the respective plurality of gene groups of an RC TME signature of the subject; and averaged gene group scores for a plurality of gene groups of other RC type B and/or RC type C samples.

In some embodiments, methods described by the disclosure further comprise identifying the subject as being “TKI-low” when the responder score is ≤0.75; “TKI-medium” when the responder score is ≥0.75 and <0.95; or “TKI-high” when the responder score is ≥0.95.

In some embodiments, a specified threshold is 0.95.

In some embodiments, methods described by the disclosure further comprise identifying a TKI for administration to the subject when the responder score of the subject is above the specified threshold or wherein the subject is identified as being “TKI-medium” or “TKI-high”.

In some embodiments, methods described by the disclosure further comprise administering a TKI to the subject when the responder score of the subject is above the specified threshold or wherein the subject is identified as being “TKI-medium” or “TKI-high”.

In some embodiments, a TKI comprises a small molecule or antibody. In some embodiments, an antibody is a monoclonal antibody.

In some embodiments, renal cancer is clear cell renal carcinoma (ccRCC).

In some embodiments, when a subject is identified as “TKI-low” using a TKI responder score, methods described herein further comprise identifying the one or more therapeutic agents as: a TKI when the subject is identified, using the IO responder score, as “IO-low”; a combination of a TKI and an IO agent when the subject is identified, using the IO responder score, as “IO-low”; or, a combination of a TKI and an IO agent when the subject is identified, using the IO responder score, as “IO-medium” or “IO-high”.

In some embodiments, when a subject is identified as “TKI-medium” using the TKI responder score, methods described by the disclosure further comprise identifying the one or more therapeutic agents as a combination of a TKI and an IO agent when the subject is identified, using the IO responder score, as “IO-high”.

In some embodiments, when a subject is identified as “TKI-high” using the TKI responder score, methods described by the disclosure further comprise identifying the one or more therapeutic agents as: a TKI when the subject is identified, using the IO responder score, as “IO-low” or “IO-medium”; or, a combination of a TKI and an IO agent when the subject is identified, using the IO responder score, as “IO-high”.

In some embodiments, methods described by the disclosure further comprise administering an identified one or more therapeutic agents to the subject.

In some embodiments, methods described by the disclosure further comprise providing a recommendation that the identified one or more therapeutic agents be administered to the subject.

### BRIEF DESCRIPTION OF DRAWINGS

FIG. 1 is a diagram depicting a flowchart of an illustrative process for determining a renal cancer (RC) tumor microenvironment (TME) type for a subject having, suspected of having, or at risk of having renal cancer, according to some embodiments of the technology as described herein.

FIG. 2 is a diagram depicting a flowchart of an illustrative process for processing sequencing data to obtain RNA expression data, according to some embodiments of the technology as described herein.

FIG. 3 is a diagram depicting an illustrative technique for determining gene group scores, according to some embodiments of the technology as described herein.

FIG. 4 is a diagram depicting an illustrative technique for identifying an RC TME type using an RC TME signature, according to some embodiments of the technology as described herein.

FIG. 5 provides an exemplary heatmap of clear cell renal carcinoma cancer (ccRCC) samples classified into five distinct RC TME types (A, B, C, D, E) using RC TME signatures comprising 33 gene group scores, according to some embodiments of the technology described herein.

FIGS. 6A-6B provide representative data, according to some embodiments of the technology described herein, indicating association of the RCT IE/F (also referred to as RC TME type A) and IE (RC TME type B) with superior clinical response (>50% response rate) in the IO+TKI (Atezolizumab+Bevacizumab) cohort. RC TME type C, characterized by fibrotic genes enrichment, responded poorly to IO containing regimen (45-67%); whereas RC TME type D, desert subtype, responded intermediately to all four regimens. Conversely, subjects having RC TME type E, characterized by elevated angiogenesis and the absence of immune cell infiltration, responded significantly better to a single agent TKI (˜80% response rate in Sunitinib).

FIG. 7 is a diagram depicting a flowchart of an illustrative process for a machine learning model for assessing likelihood of a subject's response to IO therapy, according to some embodiments of the technology described herein.

FIGS. 8A-8F provide one example of training and validation of a machine learning model for assessing likelihood of a subject's response to IO therapy, according to some embodiments of the technology described herein.

FIG. 9 is a diagram depicting a flowchart of an illustrative process for a machine learning model for assessing likelihood of a subject's response to TKI therapy, according to some embodiments of the technology described herein.

FIGS. 10A-10J provide one example of training and validation of a machine learning model for assessing likelihood of a subject's response to TKI therapy, according to some embodiments of the technology described herein.

FIGS. 11A-11E provide representative data for machine learning models for assessing likelihood of a subject's response to IO therapy or TKI therapy, according to some embodiments of the technology described herein. FIG. 11A shows data indicating 10 responder score is consistent across various datasets, whereas other biomarkers or signatures are not consistent. FIG. 11B shows data indicating TKI responder score is consistent across various datasets, whereas other biomarkers or signatures are not consistent. FIG. 11C shows representative data indicating TKI responder score shows strong associations across datasets. FIG. 11D shows representative data indicating combined IO/TKI responder scores show strong associations across datasets. FIG. 11E shows representative data indicating that IO and TKI responder scores improve prediction of median progression free survival (mPFS) and overall response rate (oRR).

FIG. 12 provides representative data relating to myogenesis signatures, according to some embodiments of the technology described herein. The figure provides a representative heatmap showing production of a myogenesis signature for clear cell renal carcinoma (ccRCC) samples based on ssGSEA analysis and median scaling of 14 genes (left), and data indicating that the 14 genes of the signature are expressed mainly in myoblasts or muscle cells.

FIGS. 13A-13B provide representative data for samples having high myogenesis signatures. FIG. 13A shows representative data showing RECIST characterization (complete response (CR), partial response (PR), stable disease (SD), and progressive disease (PD)) plotted against myogenesis signature FIG. 13B shows representative data that samples having high myogenesis signatures come from bone metastasis (MBO) patients, according to some embodiments of the technology described herein.

FIG. 14 is a diagram depicting a flowchart of an illustrative process for selecting one or more therapeutic agents for administration to a subject having renal cancer. First, an International Metastatic RCC Database Consortium (IMDC) Risk Score is generated for the subject. If the subject is identified as having a Poor IMDC Risk Score, it is determined that the subject should be administered a combination of immuno-oncology agent and a TKI. If the subject is identified as having a Favorable or Intermediate IMDC Risk Score, then an IO responder score and a TKI responder score are generated. After the responder scores are generated, one or more therapeutic agents are selected for the subject using the IO and TKI responder scores.

FIG. 15 depicts an illustrative implementation of a computer system that may be used in connection with some embodiments of the technology described herein.

### DETAILED DESCRIPTION

Aspects of the disclosure relate to methods for characterizing subjects having certain renal (kidney) cancers (RC), such as clear cell renal carcinoma (ccRCC). Clear cell renal cell carcinoma (ccRCC) is one of the most common renal cancers, and is known to have marked genetic intratumor heterogeneity (ITH). It has been found that ITH underlies tumor evolution, metastasis, and clinical responses to various therapies. Studies have focused on the utilization of whole exome sequencing to uncover evolution patterns, the mutational profile underlying ITH, clonal architecture, and inter-patient tumor differences. Multi-region DNA sequencing has revealed that del(3p) and ampl(5q), two common aberrations observed in 43% of ccRCC, are the first genetic events in kidney cell malignization. Additional DNA sequencing analyses suggest that clonal architecture can divide RC tumors into three prognostic subtypes that predict clinical outcome. Currently, immunotherapy has dramatically improved the clinical outcomes of RC patients, and the combination of ipilimumab and nivolumab is FDA-approved for frontline metastatic ccRCC. However, notable differences have been observed in patient responses to immunotherapy, which cannot be explained through genetic heterogeneity alone. Accordingly, the inventors have recognized that there is a need to develop methods for molecular characterization of RC types specifically based upon the underlying biology of both the tumor microenvironment and malignant cells, rather than more broadly defined cancer biomarkers and/or ITH.

Aspects of the disclosure relate to statistical techniques for analyzing expression data (e.g., RNA expression data), which was obtained from a biological sample obtained from a subject that has renal cancer (RC), is suspected of having RC, or is at risk of developing RC, in order to generate a gene expression signature for the subject (termed an “RC TME signature” or “RC TME signature” herein) and use this signature to identify a particular RC TME type that the subject may have. In some embodiments, the RC is ccRCC.

The inventors have recognized that a combination of certain gene group scores (e.g., a gene group scores for at least some of the gene groups listed in Table 1) may be combined to form an RC TME signature that characterizes patients having RC more accurately than previously developed methods. A RC TME signature comprising a combination of gene group scores associated with the tumor microenvironment and gene group scores associated with malignant cells, in turn, may be used to identify the subject as having a particular renal cancer (RC) tumor microenvironment (TME) type. In some embodiments, such RC TME types are useful for identifying the prognosis and/or likelihood that a subject will respond to particular therapeutic interventions (e.g., immuno-oncology (IO) agents, tyrosine kinase inhibitors (TKI), combinations of IO and TKI, etc.).

The inventors have also recognized that data relating to certain gene expression signatures (e.g., RC TME signature, myogenesis signature, etc.) of subjects having RC may be used to train machine learning-based models to produce responder scores that are reflective of a subject's likelihood of responding to treatment with certain therapeutic agents (e.g., IO agents, TKIs, combinations of IO agents and TKIs, etc.). Such responder scores are useful, in some embodiments, for guiding selection of therapeutic agents for treating RC patients by aiding in selection of therapeutic agents to which a patient has an increased likelihood of responding. The methods also aid in steering selection away from therapeutic agents to which a patient is unlikely to respond, as in the case of “clear IO non-responders” further described in the Examples.

The use of RC TME signatures comprising the combinations of gene group scores described by the disclosure represents an improvement over previously described RC molecular biomarkers or tumor microenvironment analyses because the specific groups of genes used to produce the RC TME signatures described herein better reflect the molecular tumor microenvironments of RC because these gene groups are associated with 1) immune and stromal tumor biology, and 2) renal cancer metabolic pathway activity. These focused combinations of gene groups (e.g., gene groups consisting of some or all of the genes listed in Table 1, or some or all genes of a myogenesis signature listed in Table 2) are unconventional, and differ from previously described molecular signatures, which attempt to incorporate expression data from either very large numbers of genes, or only account for certain subsets of genes involved in cancer (e.g., analysis limited only to immune cells).

The RC TME typing methods described herein have several utilities. For example, identifying a subject's RC TME type using methods described herein may allow for the subject to be diagnosed as having (or being at a high risk of developing) an aggressive form of RC at a timepoint that is not possible with previously described RC characterization methods. Earlier detection of aggressive RC types, enabled by the RC TME signatures described herein, improve the patient diagnostic technology by enabling earlier chemotherapeutic intervention for patients than currently possible for patients tested for RC using other methods.

As described herein, the inventors have also determined that subjects identified by methods described herein as having RX TME type A or RC TME type B are characterized as having a good prognosis and/or an increased likelihood of responding to certain therapeutic treatments, such as a combination of IO agents and TKIs.

Conversely, the inventors have determined that identifying a subject as having RC TME type E using methods described herein, are less likely to respond to IO agents but will likely respond to TKIs. Additionally, the inventors have determined that identifying subjects having a high myogenesis signature and/or certain other biomarkers (e.g., high ploidy, mutations in genes associated with mTOR activation or antigen presentation, etc.) are likely to be “clear IO non-responders”, and therefore should not be administered immunotherapeutic agents as a first line therapy for RC (e.g., ccRCC). Thus, the techniques developed by the inventors and described herein improve patient treatment and associated outcomes by increasing patient comfort, and avoiding toxic side effects of therapy that is not expected to be effective for the subject.

Clear Cell Renal Carcinomas (ccRCCs)

Aspects of the disclosure relate to methods of determining the renal cancer (RC) tumor microenvironment (TME) type (of a subject having, suspected of having, or at risk of having RC. As used herein, a “subject” may be a mammal, for example a human, non-human primate, rodent (e.g., rat, mouse, guinea pig, etc.), dog, cat, horse etc. In some embodiments, the subject is a human. The terms “individual” or “subject” may be used interchangeably with “patient.” As used herein, “renal cancer” refers to any renal or kidney adenocarcinoma, or any other types of malignancies caused by one or more various genetic mutations in the body that affects cells (originally present in or metastasized to) the kidneys of a subject. As used herein, “cancer” refers to any malignant and/or invasive growth or tumor caused by abnormal cell growth in a subject, including solid tumors, blood cancer, bone marrow or lymphoid cancer, etc. Examples of renal cancers include but are not limited to adenocarcinoma of the kidney(s) that are derived from proximal nephron and/or tubular epithelium of the kidney(s), for example clear cell renal cell carcinoma (ccRCC), and malignant epithelial cells with clear cytoplasm and a compact-alveolar (nested) or acinar growth pattern interspersed with intricate, arborizing vasculature.

A subject having RC may exhibit one or more signs or symptoms of RC, for example the presence of cancerous cells (e.g., tumor cells), fever, swelling, bleeding (e.g., bloody urine), nausea and vomiting, persistent lower back pain, and weight loss. In some embodiments, a subject having RC does not exhibit one or more signs or symptoms of RC. In some embodiments, a subject having RC has been diagnosed by a medical professional (e.g., licensed physician) as having RC based upon one or more assays (e.g., clinical assays, molecular diagnostics, etc.) that indicate that the subject has ccRCC, even in the absence of one or more signs or symptoms.

A subject suspected of having RC typically exhibits one or more signs or symptoms of RC, for example ccRCC. In some embodiments, a subject suspected of having RC exhibits one or more signs or symptoms of RC but has not been diagnosed by a medical professional (e.g., a licensed physician) and/or has not received a test result (e.g., a clinical assay, molecular diagnostic, etc.) indicating that the subject has RC.

A subject at risk of having RC may or may not exhibit one or more signs or symptoms of RC. In some embodiments, a subject at risk of having RC comprises one or more risk factors that increase the likelihood that the subject will develop RC. Examples of risk factors include the presence of pre-cancerous cells in a clinical sample, having one or more genetic mutations that predispose the subject to developing cancer (e.g., RC, such as ccRCC), taking one or more medications that increase the likelihood that the subject will develop cancer (e.g., RC, such as ccRCC), family history of RC, and the like.

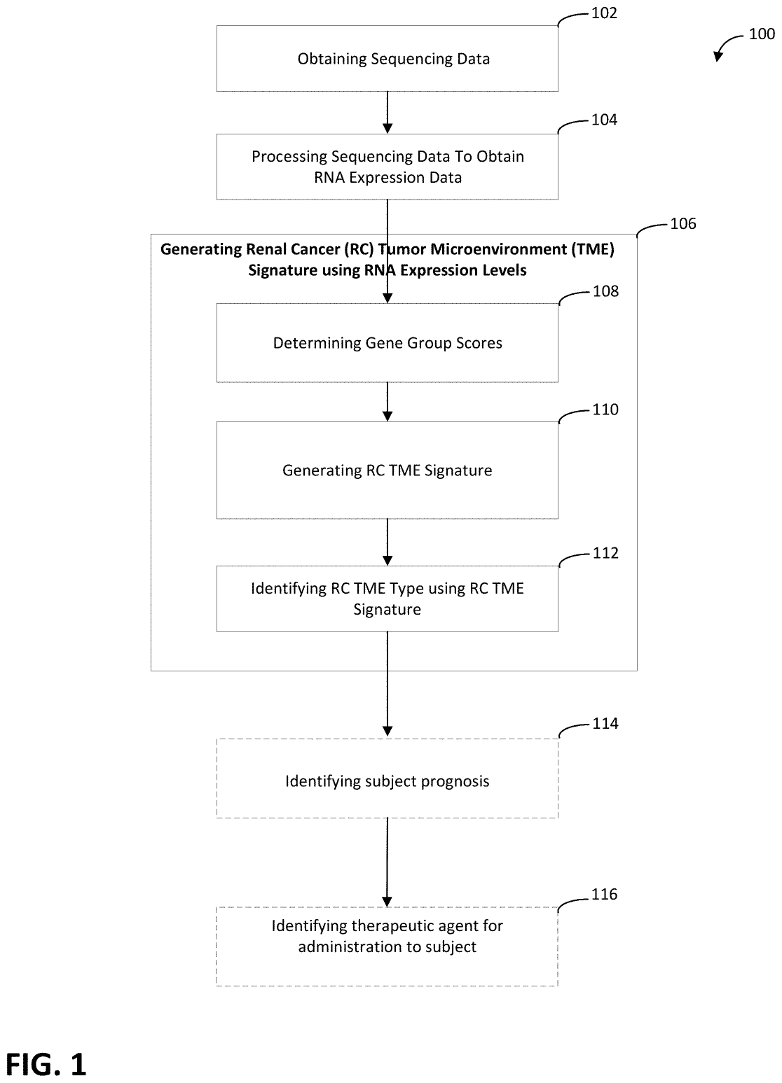

FIG. 1 is a flowchart of an illustrative process 100 for determining an RC TME signature for a subject and using the determined RC TME signature to identify the RC TME type for the subject.

Various acts of process 100 may be implemented using any suitable computing device(s). For example, in some embodiments, one or more acts of the illustrative process 100 may be implemented in a clinical or laboratory setting. For example, one or more acts of the process 100 may be implemented on a computing device that is located within the clinical or laboratory setting. In some embodiments, the computing device may directly obtain RNA expression data from a sequencing apparatus located within the clinical or laboratory setting. For example, a computing device included in the sequencing apparatus may directly obtain the RNA expression data from the sequencing apparatus. In some embodiments, the computing device may indirectly obtain RNA expression data from a sequencing apparatus that is located within or external to the clinical or laboratory setting. For example, a computing device that is located within the clinical or laboratory setting may obtain expression data via a communication network, such as Internet or any other suitable network, as aspects of the technology described herein are not limited to any particular communication network.

Additionally or alternatively, one or more acts of the illustrative process 100 may be implemented in a setting that is remote from a clinical or laboratory setting. For example, the one or more acts of process 100 may be implemented on a computing device that is located externally from a clinical or laboratory setting. In this case, the computing device may indirectly obtain RNA expression data that is generated using a sequencing apparatus located within or external to a clinical or laboratory setting. For example, the expression data may be provided to computing device via a communication network, such as Internet or any other suitable network.

It should be appreciated that, in some embodiments, not all acts of process 100, as illustrated in FIG. 1, may be implemented using one or more computing devices. For example, the act 114 of identifying the subject's prognosis may be implemented manually (e.g., by a clinician), automatically (e.g., by software identifying a RC TME type associated with a particular prognosis), or in part manually and in part automatically (e.g., a clinician may identify a RC TME type associated with a particular prognosis in part using information generated by the software, for example, using the techniques described herein). Process 100 begins at act 102 where sequencing data for a subject is obtained. In some embodiments, the sequencing data may be obtained by sequencing a biological sample (e.g., kidney biopsy and/or tumor tissue) obtained from the subject using any suitable sequencing technique. The sequencing data may include sequencing data of any suitable type, from any suitable source, and be in any suitable format. Examples of sequencing data, sources of sequencing data, and formats of sequencing data are described herein including in the section called “Obtaining RNA Expression Data”.

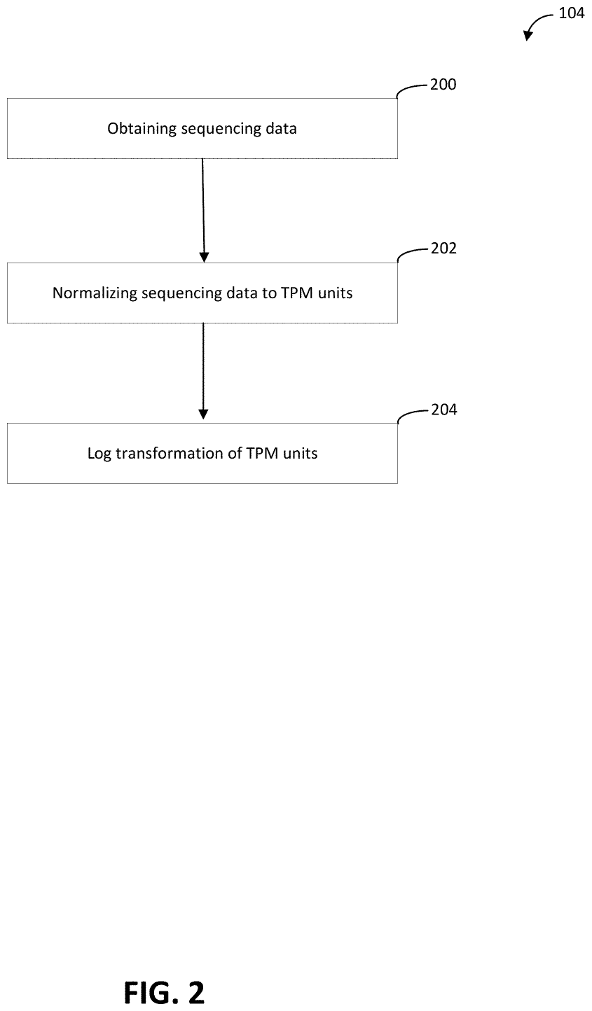

As one illustrative example, in some embodiments, the sequencing data may comprise bulk sequencing data. The bulk sequencing data may comprise at least 1 million reads, at least 5 million reads, at least 10 million reads, at least 20 million reads, at least 50 million reads, or at least 100 million reads. In some embodiments, the sequencing data comprises bulk RNA sequencing (RNA-seq) data, single cell RNA sequencing (scRNA-seq) data, or next generation sequencing (NGS) data. In some embodiments, the sequencing data comprises microarray data. Next, process 100 proceeds to act 104, where the sequencing data obtained at act 102 is processed to obtain gene expression data. This may be done in any suitable way and may involve normalizing bulk sequencing data to transcripts-per-million (TPM) units (or other units) and/or log transforming the RNA expression levels in TPM units. Converting the data to TPM units and normalization are described herein including with reference to FIG. 2.

Next, process 100 proceeds to act 106, where a renal cancer (RC) tumor microenvironment (TME) signature is generated for the subject using the RNA expression data generated at act 104 (e.g., from bulk-sequencing data, converted to TPM units and subsequently log-normalized, as described herein including with reference to FIG. 2).

As described herein, in some embodiments, an RC TME signature comprises two or more (e.g., 2, 3, 4, 5, 6, 7, 8, 9, 10, 11, 12, 13, 14, 15, 16, 17, 18, 19, 20, 21, 22, 23, 24, 25, 26, 27, etc.) gene group scores. In some embodiments, the two or more gene group scores comprise gene group scores (which may also be referred to as gene group enrichment scores or gene group expression scores) for some or all of the gene groups shown in Table 1.

Accordingly, act 106 comprises: act 108 where the gene group scores are determined, act 110 where the RC TME signature is determined, and act 112 where the RC TME type is determined by using RC TME signature. In some embodiments, determining the gene group scores comprises determining, for each of multiple (e.g., some or all of the) gene groups listed in Table 1, a respective gene score. In some embodiments, determining the gene group scores comprises determining respective gene group scores for 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or more gene groups (e.g., gene groups listed in Table 1). The gene group score for a particular gene group may be determined using RNA expression levels for at least some of the genes in the gene group (e.g., the expression levels obtained at act 104). The RNA expression levels may be processed using a gene set enrichment analysis (GSEA) technique to determine the score for the particular gene group.

In some embodiments, determining the RC TME gene signature comprises: determining gene group scores using the RNA expression levels for at least three genes from each of at least two of the gene groups, the gene groups including: (a) MHC I group: HLA-A, HLA-B, HLA-C, B2M, TAP1, TAP2, NLRC5, TAPBP; (b) MHC II group: HLA-DQA1, HLA-DMA, HLA-DRB1, HLA-DMB, CIITA, HLA-DPA1, HLA-DPB1, HLA-DRA, HLA-DQB1; and (c) Coactivation molecules group: CD28, CD40, TNFRSF4, ICOS, TNFRSF9, CD27, CD80, CD86, CD40LG, CD83, TNFSF4, ICOSLG, TNFSF9, CD70; (d) Effector cells group: IFNG, GZMA, GZMB, PRF1, GZMK, ZAP70, GNLY, FASLG, TBX21, EOMES, CD8A, CD8B; (e) T cell traffic group: CXCL9, CCL3, CXCR3, CXCL10, CXCL11, CCL5, CCL4, CX3CL1, CX3CR1; (f) NK cells group: GZMB, NKG7, CD160, GZMH, CD244, EOMES, KLRK1, NCR1, GNLY, KLRF1, FGFBP2, SH2D1B, KIR2DL4, IFNG, NCR3, KLRC2, CD226; (g) T cells group: TRAC, TRBC2, TBX21, CD3E, CD3D, ITK, TRBC1, CD3G, CD28, TRAT1, CD5; (h) B cells group: CR2, MS4A1, CD79A, FCRL5, STAP1, TNFRSF17, TNFRSF13B, CD19, BLK, CD79B, TNFRSF13C, CD22, PAX5; (i) M1 signature group: IL1B, IL12B, NOS2, SOCS3, IRF5, IL23A, TNF, IL12A, CMKLR1; (j) Th1 signature group: IL12RB2, IL2, TBX21, IFNG, STAT4, IL21, CD40LG; (k) Antitumor cytokines group: IFNA2, CCL3, TNF, TNFSF10, IL21, IFNB1; (l) Checkpoint inhibition group: CTLA4, HAVCR2, CD274, LAG3, BTLA, VSIR, PDCD1LG2, TIGIT, PDCD1; (m) Treg group: TNFRSF18, IKZF2, IL10, IKZF4, CTLA4, FOXP3, CCR8; (n) T reg traffic group: CCL28, CCR10, CCR4, CCR8, CCL17, CCL22, CCL1; (o) Neutrophil signature group: FCGR3B, CD177, CTSG, PGLYRP1, FFAR2, CXCR2, PRTN3, ELANE, MPO, CXCR1; (p) Granulocyte traffic group: CXCL8, CCR3, CXCR2, CXCL2, CCL11, KITLG, CXCL1, CXCL5, CXCR1; (q) MDSC group: IDO1, ARG1, IL10, CYBB, PTGS2, IL4I1, IL6; (r) MDSC traffic group: CCL15, IL6R, CSF2RA, CSF2, CXCL8, CXCL12, IL6, CSF3, CCL26, CXCR4, CXCR2, CSF3R, CSF1, CXCL5, CSF1R; (s) Macrophage group: MRC1, CD163, MSR1, SIGLEC1, IL4I1, CD68, IL10, CSF1R; (t) Macrophage DC traffic group: CCL7, CCL2, XCR1, XCL1, CSF1, CCR2, CCL8, CSF1R; (u) Th2 signature group: IL13, CCR4, IL10, IL5, IL4; (v) Protumor cytokines group: MIF, TGFB1, IL10, TGFB3, IL6, TGFB2, IL22; (w) Cancer associated fibroblast (CAF) group: PDGFRB, COL6A3, FBLN1, CXCL12, COL6A2, COL6A1, LUM, CD248, COL5A1, MMP2, COL1A1, MFAP5, PDGFRA, LRP1, FGF2, MMP3, FAP, COL1A2, ACTA2; (x) Matrix group: COL11A1, LAMB3, FN1, COL1A1, COL4A1, ELN, LGALS9, LGALS7, LAMC2, TNC, LAMA3, COL3A1, COL5A1, VTN, COL1A2; (y) Matrix-remodeling group: MMP1, PLOD2, MMP2, MMP12, ADAMTS5, ADAMTS4, LOX, MMP9, MMP11, MMP3, MMP1, CA9; (z) Angiogenesis group: VEGFA, VEGFB, VEGFC, PDGFC, CXCL8, CXCR2, FLT1, PGF, KDR, ANGPT1, ANGPT2, TEK, VWF, CDH5; (aa) endothelium group: NOS3, MMRN1, FLT1, CLEC14A, MMRN2, VCAM1, ENG, VWF, CDH5, KDR; (bb) Proliferation rate group: MKI67, ESCO2, CETN3, CDK2, CCND1, CCNE1, AURKA, AURKB, E2F1, MYBL2, BUB1, CCNB1, MCM2, MCM6; (cc) EMT signature group: SNAI2, TWIST1, ZEB2, SNAI1, ZEB1, TWIST2, CDH2; (dd) Cyclic Nucleotides Metabolism group: ADCY4, PDE11A, PDE6A, PDE9A, PDE6C, ADCY7, PDE4A, PDE8A, PDE1B, PDE1A, GUCY2C, GUCY1A3, ADCY9, ADCY2, PDE6B, ADCY8, PDE8B, GUCY2F, PDE4C, PDE3A, GUCY1A2, PDE6G, PDE1C, GUCY2D, ADCY10, GUCY1B3, GUCY1B2, PDE7B, PDE5A, PDE6D, NPR2, ADCY5, NPR1, ADCY6, PDE7A, PDE2A, PDE4B, PDE10A, PDE6H, PDE4D, ADCY1, PDE3B, ADCY3; (ee) Glycolysis and Gluconeogenesis group: SLC2A9, PFKL, GCK, PFKFB4, SLC16A7, PCK1, PGAM2, GAPDH, BPGM, G6PC2, FBP2, LDHD, SLC2A3, GPI, ENO1, SLC25A11, PFKFB3, PFKM, LDHAL6B, SLC2A2, G6PC3, SLC2A6, GAPDHS, SLC2A11, PCK2, PFKP, PGK1, ALDOC, SLC2A10, ACYP2, SLC2A4, PKLR, HKDC1, PGK2, SLC2A8, PGAM1, SLC5A1, SLC5A12, SLC16A1, ALDOB, HK3, HK1, SLC5A9, GPD2, PFKFB1, SLC2A7, SLC5A11, SLC5A3, ACYP1, SLC16A8, PFKFB2, ALDOA, SLC5A2, HK2, ENO3, SLC2A12, FBP1, LDHA, LDHB, LDHC, G6PC, SLC2A14, SLC5A8, TPI1, SLC16A3, PKM2, ENO2, PGM1, UEVLD, LDHAL6A, SLC2A1, PGM2; (xx) Citric Acid Cycle group: ACLY, FAH, PC, MDH1B, SLC16A7, IREB2, PCK1, MDH1, SLC33A1, ALDH1B1, IDH3B, DLST, PDHB, MDH2, ACO1, IDH1, SLC5A6, HICDH, SLC16A8, GOT1, ME3, ME1, CS, OGDH, SDHA, ALDH5A1, CLYBL, SDHD, IDH3A, SLC25A1, ACSS2, SDHC, ACSS1, SUCLA2, SLC13A5, PDHX, SDHB, ALDH4A1, PCK2, DLD, ACO2, PDHA1, SLC13A2, FAHD1, IDH2, GOT2, ME2, ADSL, SUCLG2, SLC13A3, SUCLG1, SLC25A10, FH, IDH3G, SLC16A1, SLC25A11, PDHA2, DLAT; and (ff) Fatty Acid Metabolism group: MLYCD, ALDH3A2, SLC27A5, SLC27A3, LIPC, SLC27A2, ACSL4, ACSL1, PCCB, SLC25A20, AADAC, SLC22A4, SLC22A5, ECH1, PCCA, SLC27A1, SLC27A4, CROT, ACSL5, ACSL3, CYP4F12.

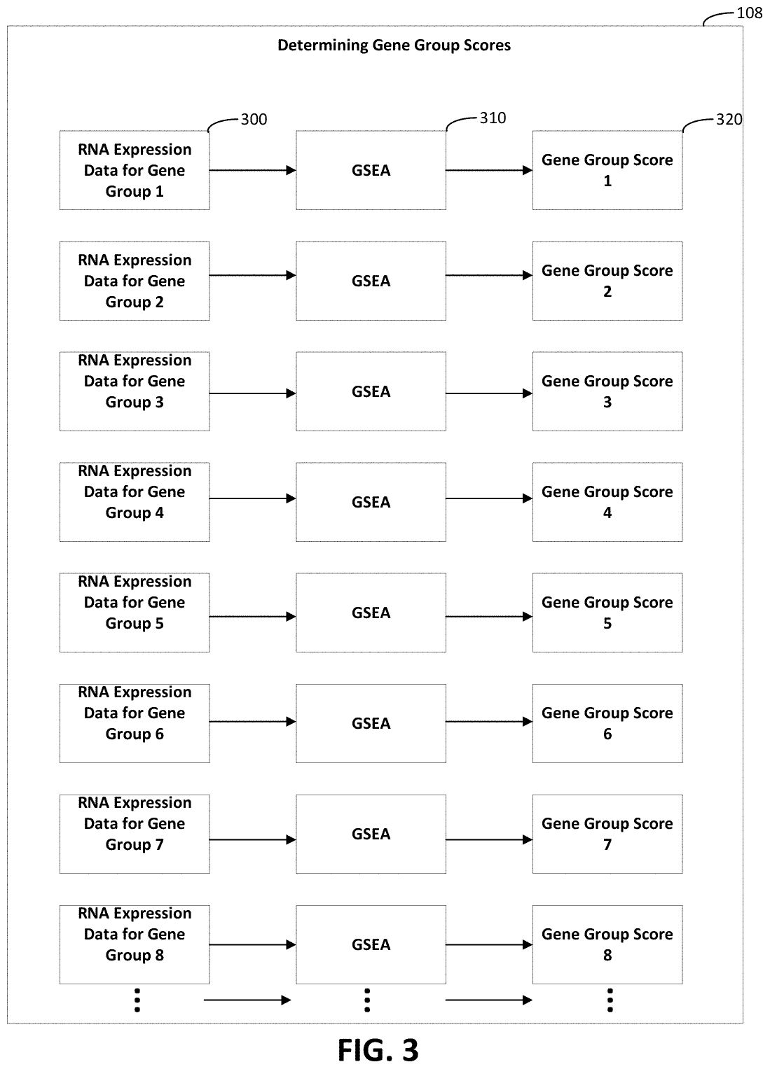

Aspects of determining the gene group enrichment scores are described herein, including with reference to FIG. 3 and in the Section titled “Gene Expression Signatures”.

As described above, at act 110, the RC TME signature is produced. In some embodiments, the RC TME signature consists of only gene group scores for one or more (e.g., all) gene groups listed in Table 1. In some embodiments, the RC TME signature comprises gene group scores for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 gene groups listed in Table 1. In some embodiments, each gene group score is determined using RNA expression levels of some or all (e.g., at least 2, 3, 4, 5, 6, 7, 8, 9, 10, 11, 12, 13, 14, 15, 16, 17, 18, 19, 20, etc.) of the genes of each gene group listed in Table 1.

Next, process 100 proceeds to act 112, where an RC TME type is identified for the subject using the RC TME signature generated at act 110. This may be done in any suitable way. For example, in some embodiments, the each of the possible RC TME types is associated with a respective plurality of RC TME signature clusters. In such embodiments, an RC TME type for the subject may be identified by associating the RC TME signature of the subject with a particular one of the plurality of RC TME signature clusters; and identifying the RC TME type for the subject as the RC TME type corresponding to the particular one of the plurality of RC TME signature clusters to which the RC TME signature of the subject is associated. Examples of RC TME types are described herein. Aspects of identifying an RC TME type for a subject are described herein including in the section below titled “Generating RC TME Signature and Identifying TME Type”. In some embodiments, process 100 completes after act 112 completes. In some such embodiments the determined RC TME signature and/or identified RC TME Type may be stored for subsequent use, provided to one or more recipients (e.g., a clinician, a researcher, etc.), and/or used to update the RC TME signature clusters (as described hereinbelow).

However, in some embodiments, one or more other acts are performed after act 112. For example, in the illustrated embodiment, a subject's prognosis may be identified based on the RC TME type determined for the subject. For example, in some embodiments, the subject is identified (at act 114) as having a good prognosis when the subject is identified as having RC TME type D or RC TME type E. Subsequently, or as an alternative to act 114, process 100 may proceed to act 116, where the subject's RC TME type identified in act 112 is used to identify (or recommend) a therapeutic agent for administration to the subject. For example, a subject may be identified as having an increased likelihood of responding to a TKI when the subject is identified as having RC type E.

In some embodiments, process 100 completes after act 114 or 116 completes. In some such embodiments the determined RC TME signature and/or identified RC TME Type may be stored for subsequent use, provided to one or more recipients (e.g., a clinician, a researcher, etc.), and/or used to update the RC TME signature clusters (as described hereinbelow).

### Biological Samples

Aspects of the disclosure relate to methods for determining an RC TME type of a subject by obtaining sequencing data from a biological sample that has been obtained from the subject.

The biological sample may be from any source in the subject's body including, but not limited to, any fluid such as blood (e.g., whole blood, blood serum, or blood plasma), lymph node, and kidney(s). Other source in the subject's body may be from saliva, tears, synovial fluid, cerebrospinal fluid, pleural fluid, pericardial fluid, ascitic fluid, and/or urine], hair, skin (including portions of the epidermis, dermis, and/or hypodermis), oropharynx, laryngopharynx, esophagus, bronchus, salivary gland, tongue, oral cavity, nasal cavity, vaginal cavity, anal cavity, stomach, intestine, bone, bone marrow, brain, thymus, spleen, appendix, colon, rectum, anus, liver, biliary tract, pancreas, ureter, bladder, urethra, uterus, vagina, vulva, ovary, cervix, scrotum, penis, prostate, testicle, seminal vesicles, and/or any type of tissue (e.g., muscle tissue, epithelial tissue, connective tissue, or nervous tissue).

The biological sample may be any type of sample including, for example, a sample of a bodily fluid, one or more cells, one or more pieces of tissue(s) or organ(s). In some embodiments, the biological sample comprises kidney tissue sample of the subject. Examples of kidney tissue samples include but are not limited to glomerulus parietal cells, glomerulus podocytes, proximal tubule brush border cells, loop of Henle thin segment cells, thick ascending limb cells, distal tubule cells, collecting duct principal cells, collecting duct intercalated cells, interstitial kidney cells, and kidney tumor cells.

In some embodiments, a kidney tissue sample may be obtained from a subject using a surgical procedure (e.g., laparoscopic surgery, microscopically controlled surgery, or endoscopy), punch biopsy, endoscopic biopsy, or needle biopsy (e.g., a fine-needle aspiration, core needle biopsy, vacuum-assisted biopsy, or image-guided biopsy).

A sample of lymph node or blood, in some embodiments, refers to a sample comprising cells, e.g., cells from a blood sample or lymph node sample. In some embodiments, the sample comprises non-cancerous cells. In some embodiments, the sample comprises pre-cancerous cells. In some embodiments, the sample comprises cancerous cells. In some embodiments, the sample comprises blood cells. In some embodiments, the sample comprises lymph node cells. In some embodiments, the sample comprises lymph node cells and blood cells.

A sample of blood may be a sample of whole blood or a sample of fractionated blood. In some embodiments, the sample of blood comprises whole blood. In some embodiments, the sample of blood comprises fractionated blood. In some embodiments, the sample of blood comprises buffy coat. In some embodiments, the sample of blood comprises serum. In some embodiments, the sample of blood comprises plasma. In some embodiments, the sample of blood comprises a blood clot.

In some embodiments, a sample of blood is collected to obtain the cell-free nucleic acid (e.g., cell-free DNA) in the blood.

In some embodiments, the sample may be from a cancerous tissue or an organ or a tissue or organ suspected of having one or more cancerous cells. In some embodiments, the sample may be from a healthy (e.g., non-cancerous) tissue or organ. In some embodiments, a sample from a subject (e.g., a biopsy from a subject) may include both healthy and cancerous cells and/or tissue. In certain embodiments, one sample will be taken from a subject for analysis. In some embodiments, more than one (e.g., 2, 3, 4, 5, 6, 7, 8, 9, 10, 11, 12, 13, 14, 15, 16, 17, 18, 19, 20, or more) samples may be taken from a subject for analysis. In some embodiments, one sample from a subject will be analyzed. In certain embodiments, more than one (e.g., 2, 3, 4, 5, 6, 7, 8, 9, 10, 11, 12, 13, 14, 15, 16, 17, 18, 19, 20, or more) samples may be analyzed. If more than one sample from a subject is analyzed, the samples may be procured at the same time (e.g., more than one sample may be taken in the same procedure), or the samples may be taken at different times (e.g., during a different procedure including a procedure 1, 2, 3, 4, 5, 6, 7, 8, 9, 10 days; 1, 2, 3, 4, 5, 6, 7, 8, 9, 10 weeks; 1, 2, 3, 4, 5, 6, 7, 8, 9, 10 months, 1, 2, 3, 4, 5, 6, 7, 8, 9, 10 years, or 1, 2, 3, 4, 5, 6, 7, 8, 9, 10 decades after a first procedure). A second or subsequent sample may be taken or obtained from the same region (e.g., from the same tumor or area of tissue) or a different region (including, e.g., a different tumor). A second or subsequent sample may be taken or obtained from the subject after one or more treatments, and may be taken from the same region or a different region. As a non-limiting example, the second or subsequent sample may be useful in determining whether the cancer in each sample has different characteristics (e.g., in the case of samples taken from two physically separate tumors in a patient) or whether the cancer has responded to one or more treatments (e.g., in the case of two or more samples from the same tumor prior to and subsequent to a treatment).

Any of the biological samples described herein may be obtained from the subject using any known technique. See, for example, the following publications on collecting, processing, and storing biological samples, each of which is incorporated by reference herein in its entirety: Biospecimens and biorepositories: from afterthought to science by Vaught et al. (Cancer Epidemiol Biomarkers Prev. 2012 February; 21(2):253-5), and Biological sample collection, processing, storage and information management by Vaught and Henderson (IARC Sci Publ. 2011; (163):23-42).

Any of the biological samples from a subject described herein may be stored using any method that preserves stability of the biological sample. In some embodiments, preserving the stability of the biological sample means inhibiting components (e.g., DNA, RNA, protein, or tissue structure or morphology) of the biological sample from degrading until they are measured so that when measured, the measurements represent the state of the sample at the time of obtaining it from the subject. In some embodiments, a biological sample is stored in a composition that is able to penetrate the same and protect components (e.g., DNA, RNA, protein, or tissue structure or morphology) of the biological sample from degrading. As used herein, degradation is the transformation of a component from one form to another form such that the first form is no longer detected at the same level as before degradation.

In some embodiments, the biological sample is stored using cryopreservation. Non-limiting examples of cryopreservation include, but are not limited to, step-down freezing, blast freezing, direct plunge freezing, snap freezing, slow freezing using a programmable freezer, and vitrification. In some embodiments, the biological sample is stored using lyophilization. In some embodiments, a biological sample is placed into a container that already contains a preservant (e.g., RNALater to preserve RNA) and then frozen (e.g., by snap-freezing), after the collection of the biological sample from the subject. In some embodiments, such storage in frozen state is done immediately after collection of the biological sample. In some embodiments, a biological sample may be kept at either room temperature or 4° C. for some time (e.g., up to an hour, up to 8 h, or up to 1 day, or a few days) in a preservant or in a buffer without a preservant, before being frozen.

Non-limiting examples of preservants include formalin solutions, formaldehyde solutions, RNALater or other equivalent solutions, TriZol or other equivalent solutions, DNA/RNA Shield or equivalent solutions, EDTA (e.g., Buffer AE (10 mM TrisCl; 0.5 mM EDTA, pH 9.0)) and other coagulants, and Acids Citrate Dextronse (e.g., for blood specimens).

In some embodiments, special containers may be used for collecting and/or storing a biological sample. For example, a vacutainer may be used to store blood. In some embodiments, a vacutainer may comprise a preservant (e.g., a coagulant, or an anticoagulant). In some embodiments, a container in which a biological sample is preserved may be contained in a secondary container, for the purpose of better preservation, or for the purpose of avoid contamination.

Any of the biological samples from a subject described herein may be stored under any condition that preserves stability of the biological sample. In some embodiments, the biological sample is stored at a temperature that preserves stability of the biological sample. In some embodiments, the sample is stored at room temperature (e.g., 25° C.). In some embodiments, the sample is stored under refrigeration (e.g., 4° C.). In some embodiments, the sample is stored under freezing conditions (e.g., −20° C.). In some embodiments, the sample is stored under ultralow temperature conditions (e.g., −50° C. to −800° C.). In some embodiments, the sample is stored under liquid nitrogen (e.g., −1700° C.). In some embodiments, a biological sample is stored at −60° C. to −8-° C. (e.g., −70° C.) for up to 5 years (e.g., up to 1 month, up to 2 months, up to 3 months, up to 4 months, up to 5 months, up to 6 months, up to 7 months, up to 8 months, up to 9 months, up to 10 months, up to 11 months, up to 1 year, up to 2 years, up to 3 years, up to 4 years, or up to 5 years). In some embodiments, a biological sample is stored as described by any of the methods described herein for up to 20 years (e.g., up to 5 years, up to 10 years, up to 15 years, or up to 20 years).

### Obtaining RNA Expression Data

Aspects of the disclosure relate to methods of determining an RC TME type of a subject using sequencing data or RNA expression data obtained from a biological sample from the subject.

The sequencing data may be obtained from the biological sample using any suitable sequencing technique and/or apparatus. In some embodiments, the sequencing apparatus used to sequence the biological sample may be selected from any suitable sequencing apparatus known in the art including, but not limited to, Illumina™, SOLid™, Ion Torrent™, PacBio™, a nanopore-based sequencing apparatus, a Sanger sequencing apparatus, or a 454™ sequencing apparatus. In some embodiments, sequencing apparatus used to sequence the biological sample is an Illumina sequencing (e.g., NovaSeq™, NextSeq™, HiSeq™, MiSeq™, or MiniSeq™) apparatus.

After the sequencing data is obtained, it is processed in order to obtain the RNA expression data. RNA expression data may be acquired using any method known in the art including, but not limited to: whole transcriptome sequencing, whole exome sequencing, total RNA sequencing, mRNA sequencing, targeted RNA sequencing, RNA exome capture sequencing, next generation sequencing, and/or deep RNA sequencing. In some embodiments, RNA expression data may be obtained using a microarray assay.

In some embodiments, the sequencing data is processed to produce RNA expression data. In some embodiments, RNA sequence data is processed by one or more bioinformatics methods or software tools, for example RNA sequence quantification tools (e.g., Kallisto) and genome annotation tools (e.g., Gencode v23), in order to produce expression data. The Kallisto software is described in Nicolas L Bray, Harold Pimentel, Pall Melsted and Lior Pachter, Near-optimal probabilistic RNA-seq quantification, Nature Biotechnology 34, 525-527 (2016), doi:10.1038/nbt.3519, which is incorporated by reference in its entirety herein.

In some embodiments, microarray expression data is processed using a bioinformatics R package, such as “affy” or “limma”, in order to produce expression data. The “affy” software is described in Bioinformatics. 2004 Feb. 12; 20(3):307-15. doi: 10.1093/bioinformatics/btg405. “affy—analysis of Affymetrix GeneChip data at the probe level” by Laurent Gautier 1, Leslie Cope, Benjamin M Bolstad, Rafael A Irizarry PMID: 14960456 DOI: 10.1093/bioinformatics/btg405, which is incorporated by reference herein in its entirety. The “limma” software is described in Ritchie M E, Phipson B, Wu D, Hu Y, Law C W, Shi W, Smyth G K “limma powers differential expression analyses for RNA-sequencing and microarray studies.” Nucleic Acids Res. 2015 Apr. 20; 43(7):e47. 20. https://doi.org/10.1093/nar/gkv007

PMID: 25605792, PMCID: PMC4402510, which is incorporated by reference herein its entirety.

In some embodiments, sequencing data and/or expression data comprises more than 5 kilobases (kb). In some embodiments, the size of the obtained RNA data is at least 10 kb. In some embodiments, the size of the obtained RNA sequencing data is at least 100 kb. In some embodiments, the size of the obtained RNA sequencing data is at least 500 kb. In some embodiments, the size of the obtained RNA sequencing data is at least 1 megabase (Mb). In some embodiments, the size of the obtained RNA sequencing data is at least 10 Mb. In some embodiments, the size of the obtained RNA sequencing data is at least 100 Mb. In some embodiments, the size of the obtained RNA sequencing data is at least 500 Mb. In some embodiments, the size of the obtained RNA sequencing data is at least 1 gigabase (Gb). In some embodiments, the size of the obtained RNA sequencing data is at least 10 Gb. In some embodiments, the size of the obtained RNA sequencing data is at least 100 Gb. In some embodiments, the size of the obtained RNA sequencing data is at least 500 Gb.

In some embodiments, the expression data is acquired through bulk RNA sequencing. Bulk RNA sequencing may include obtaining expression levels for each gene across RNA extracted from a large population of input cells (e.g., a mixture of different cell types.) In some embodiments, the expression data is acquired through single cell sequencing (e.g., scRNA-seq). Single cell sequencing may include sequencing individual cells.

In some embodiments, bulk sequencing data comprises at least 1 million reads, at least 5 million reads, at least 10 million reads, at least 20 million reads, at least 50 million reads, or at least 100 million reads. In some embodiments, bulk sequencing data comprises between 1 million reads and 5 million reads, 3 million reads and 10 million reads, 5 million reads and 20 million reads, 10 million reads and 50 million reads, 30 million reads and 100 million reads, or 1 million reads and 100 million reads (or any number of reads including, and between).

In some embodiments, the expression data comprises next-generation sequencing (NGS) data. In some embodiments, the expression data comprises microarray data.

Expression data (e.g., indicating expression levels) for a plurality of genes may be used for any of the methods or compositions described herein. The number of genes which may be examined may be up to and inclusive of all the genes of the subject. In some embodiments, expression levels may be determined for all of the genes of a subject. As a non-limiting example, four or more, five or more, six or more, seven or more, eight or more, nine or more, ten or more, eleven or more, twelve or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 35 or more, 40 or more, 50 or more, 60 or more, 70 or more, 80 or more, 90 or more, 100 or more, 125 or more, 150 or more, 175 or more, 200 or more, 225 or more, 250 or more, 275 or more, or 300 or more genes may be used for any evaluation described herein. As another set of non-limiting examples, the expression data may include, for each gene group listed in Table 1, expression data for at least 5, at least 10, at least 15, at least 20, at least 25, at least 35, at least 50, at least 75, at least 100 genes selected from each gene group.

In some embodiments, RNA expression data is obtained by accessing the RNA expression data from at least one computer storage medium on which the RNA expression data is stored. Additionally or alternatively, in some embodiments, RNA expression data may be received from one or more sources via a communication network of any suitable type. For example, in some embodiment, the RNA expression data may be received from a server (e.g., a SFTP server, or Illumina BaseSpace).

The RNA expression data obtained may be in any suitable format, as aspects of the technology described herein are not limited in this respect. For example, in some embodiments, the RNA expression data may be obtained in a text-based file (e.g., in a FASTQ, FASTA, BAM, or SAM format). In some embodiments, a file in which sequencing data is stored may contains quality scores of the sequencing data. In some embodiments, a file in which sequencing data is stored may contain sequence identifier information.

Expression data, in some embodiments, includes gene expression levels. Gene expression levels may be detected by detecting a product of gene expression such as mRNA and/or protein. In some embodiments, gene expression levels are determined by detecting a level of a mRNA in a sample. As used herein, the terms “determining” or “detecting” may include assessing the presence, absence, quantity and/or amount (which can be an effective amount) of a substance within a sample, including the derivation of qualitative or quantitative concentration levels of such substances, or otherwise evaluating the values and/or categorization of such substances in a sample from a subject.

FIG. 2 shows an exemplary process 104 for processing sequencing data to obtain RNA expression data from sequencing data. Process 104 may be performed by any suitable computing device or devices, as aspects of the technology described herein are not limited in this respect. For example, process 104 may be performed by a computing device part of a sequencing platform. In other embodiments, process 104 may be performed by one or more computing devices external to the sequencing platform.

Process 104 begins at act 200, where sequencing data is obtained from a biological sample obtained from a subject. The sequencing data is obtained by any suitable method, for example, using any of the methods described herein including in the Section titled “Biological Samples”.

In some embodiments, the bulk sequencing data obtained at act 104 comprises RNA-seq data. In some embodiments, the biological sample comprises blood or tissue. In some embodiments, the biological sample comprises one or more tumor cells, for example, one or more RC tumor cells.

Next, process 104 proceeds to act 202 where the sequencing data obtained at act 200 is normalized to transcripts per kilobase million (TPM) units. The normalization may be performed using any suitable software and in any suitable way. For example, in some embodiments, TPM normalization may be performed according to the techniques described in Wagner et al. (Theory Biosci. (2012) 131:281-285), which is incorporated by reference herein in its entirety. In some embodiments, the TPM normalization may be performed using a software package, such as, for example, the gcrma package. Aspects of the gcrma package are described in Wu J, Gentry RIwcfJMJ (2021). “gcrma: Background Adjustment Using Sequence Information. R package version 2.66.0.”, which is incorporated by reference in its entirety herein. In some embodiments, RNA expression level in TPM units for a particular gene may be calculated according to the following formula:

$$A \cdot \frac{1}{\sum ( A )} \cdot {10}^{6}$$

$$Where A = \frac{total reads mapped to gene \cdot {10}^{3}}{gene length in bp}$$

Next, process 104 proceeds to act 204, where the RNA expression levels in TPM units (as determined at act 202) may be log transformed. Process 104 is illustrative and there are variations. For example, in some embodiments, one or both of acts 202 and 204 may be omitted. Thus, in some embodiments, the RNA expression levels may not be normalized to transcripts per million units and may, instead, be converted to another type of unit (e.g., reads per kilobase million (RPKM) or fragments per kilobase million (FPKM) or any other suitable unit). Additionally or alternatively, in some embodiments, the log transformation may be omitted. Instead, no transformation may be applied in some embodiments, or one or more other transformations may be applied in lieu of the log transformation.

Expression data obtained by process 104 can include the sequence data generated by a sequencing protocol (e.g., the series of nucleotides in a nucleic acid molecule identified by next-generation sequencing, sanger sequencing, etc.) as well as information contained therein (e.g., information indicative of source, tissue type, etc.) which may also be considered information that can be inferred or determined from the sequence data. In some embodiments, expression data obtained by process 104 can include information included in a FASTA file, a description and/or quality scores included in a FASTQ file, an aligned position included in a BAM file, and/or any other suitable information obtained from any suitable file.

### Gene Expression Signatures

Aspects of the disclosure relate to processing of expression data to determine one or more gene expression signatures (e.g., an RC TME signature). In some embodiments, expression data (e.g., RNA expression data) is processed using a computing device to determine the one or more gene expression signatures. In some embodiments, the computing device may be operated by a user such as a doctor, clinician, researcher, patient, or other individual. For example, the user may provide the expression data as input to the computing device (e.g., by uploading a file), and/or may provide user input specifying processing or other methods to be performed using the expression data.

In some embodiments, expression data may be processed by one or more software programs running on computing device.

In some embodiments, methods described herein comprise an act of determining the RC TME signature comprising gene group scores for respective gene groups in the plurality of gene groups. In some embodiments, the RC TME signature comprises gene group scores for at least one (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33) of the gene groups listed in Table 1.

The number of genes in a gene group used to determine a gene group score may vary. In some embodiments, all RNA expression levels for all genes in a particular gene group may be used to determine a gene group score for the particular gene group. In other embodiments, RNA expression data for fewer than all genes may be used (e.g., RNA expression levels for at least two genes, at least three genes, at least five genes, between 2 and 10 genes, between 5 and 15 genes, between 3 and 30 genes, or any other suitable range within these ranges).

In some embodiments, an RC TME signature comprises a gene group score for the MHC I gene group. In some embodiments, this gene group score may be calculated using RNA expression levels of at least three genes (e.g., at least three genes, at least four genes, at least five genes, at least six genes, or at least seven genes) in the MHC I gene group, which is defined by its constituent genes: HLA-A, HLA-B, HLA-C, B2M, TAP1, TAP2, NLRC5, TAPBP.

In some embodiments, an RC TME signature comprises a gene group score for the MHC II gene group. In some embodiments, this gene group score may be calculated using RNA expression levels of at least three genes (e.g., at least three genes, at least four genes, at least five genes, at least six genes, at least seven genes, at least eight genes) in the MHC II gene group, which is defined by its constituent gene: HLA-DQA1, HLA-DMA, HLA-DRB1, HLA-DMB, CIITA, HLA-DPA1, HLA-DPB1, HLA-DRA, HLA-DQB1.

In some embodiments, an RC TME signature comprises a gene group score for the Coactivation molecules group. In some embodiments, this gene group score may be calculated using RNA expression levels of at least three genes (e.g., at least three genes, at least four genes, at least five genes, at least six genes, at least seven genes, at least eight genes, at least nine genes, or at least ten genes) in the Coactivation molecules group, which is defined by its constituent genes: CD28, CD40, TNFRSF4, ICOS, TNFRSF9, CD27, CD80, CD86, CD40LG, CD83, TNFSF4, ICOSLG, TNFSF9, CD70.

In some embodiments, an RC TME signature comprises a gene group score for the Effector cells group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than ten genes) in the Effector cells group, which is defined by its constituent genes: IFNG, GZMA, GZMB, PRF1, GZMK, ZAP70, GNLY, FASLG, TBX21, EOMES, CD8A, CD8B.

In some embodiments, an RC TME signature comprises a gene group score for the T cell traffic group. In some embodiments, this gene group score may be calculated using RNA expression levels of at least three genes (e.g., at least three genes, at least four genes, at least five genes, at least six genes, at least seven genes, or at least eight genes) in the T cell traffic group, which is defined by its constituent genes: CXCL9, CCL3, CXCR3, CXCL10, CXCL11, CCL5, CCL4, CX3CL1, CX3CR1.

In some embodiments, an RC TME signature comprises a gene group score for the NK cells group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than ten genes) in the NK cells group, which is defined by its constituent genes: GZMB, NKG7, CD160, GZMH, CD244, EOMES, KLRK1, NCR1, GNLY, KLRF1, FGFBP2, SH2D1B, KIR2DL4, IFNG, NCR3, KLRC2, CD226.

In some embodiments, an RC TME signature comprises a gene group score for the T cells group. In some embodiments, this gene group score may be calculated using RNA expression levels of at least three genes (e.g., at least three genes, at least four genes, at least five genes, at least six genes, at least seven genes, at least eight genes, at least nine genes, or at least ten genes) in the T cells group, which is defined by its constituent genes: TRAC, TRBC2, TBX21, CD3E, CD3D, ITK, TRBC1, CD3G, CD28, TRAT1, CD5.

In some embodiments, an RC TME signature gene group comprises a score for the B cells group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than ten genes) in the B cells group, which is defined by its constituent genes: CR2, MS4A1, CD79A, FCRL5, STAP1, TNFRSF17, TNFRSF13B, CD19, BLK, CD79B, TNFRSF13C, CD22, PAX5.

In some embodiments, an RC TME signature comprises a gene group score for the M1 signature group. In some embodiments, this gene group score may be calculated using RNA expression levels of at least three genes (e.g., at least three genes, at least four genes, at least five genes, at least six genes, at least seven genes, or at least eight genes) in the M1 signature group, which is defined by its constituent genes: IL1B, IL12B, NOS2, SOCS3, IRF5, IL23A, TNF, IL12A, CMKLR1.

In some embodiments, an RC TME signature comprises a gene group score for the Th1 signature group. In some embodiments, this gene group score may be calculated using RNA expression levels of at least three genes (e.g., at least three genes, at least four genes, or at least five genes) in the Th1 signature group, which is defined by its constituent genes: IL12RB2, IL2, TBX21, IFNG, STAT4, IL21, CD40LG.

In some embodiments, an RC TME signature comprises a gene group score for the Antitumor cytokines group. In some embodiments, this gene group score may be calculated using RNA expression levels of at least three genes (e.g., at least three genes, at least four genes, or at least five genes) in the Antitumor cytokines group, which is defined by its constituent genes: IFNA2, CCL3, TNF, TNFSF10, IL21, IFNB1.

In some embodiments, an RC TME signature comprises a gene group score for the Checkpoint inhibition group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than ten genes) in the Checkpoint inhibition group, which is defined by its constituent genes: CTLA4, HAVCR2, CD274, LAG3, BTLA, VSIR, PDCD1LG2, TIGIT, PDCD1.

In some embodiments, an RC TME signature comprises a gene group score for the Treg group. In some embodiments, this gene group score may be calculated using RNA expression levels of at least three genes (e.g., at least three genes, at least four genes, at least five genes, or at least six genes) in the Treg group, which is defined by its constituent genes: TNFRSF18, IKZF2, IL10, IKZF4, CTLA4, FOXP3, CCR8.

In some embodiments, an RC TME signature comprises a gene group score for the T reg traffic group. In some embodiments, this gene group score may be calculated using RNA expression levels of at least three genes (e.g., at least three genes, at least four genes, at least five genes, or at least six genes) in the T reg traffic group, which is defined by its constituent genes: CCL28, CCR10, CCR4, CCR8, CCL17, CCL22, CCL1.

In some embodiments, an RC TME signature comprises a gene group score for the Neutrophil signature group. In some embodiments, this gene group score may be calculated using RNA expression levels of at least three genes (e.g., at least three genes, at least four genes, at least five genes, at least six genes, at least seven genes, at least eight genes, or at least nine genes) in the Neutrophil group, which is defined by its constituent genes: FCGR3B, CD177, CTSG, PGLYRP1, FFAR2, CXCR2, PRTN3, ELANE, MPO, CXCR1.

In some embodiments, an RC TME signature comprises a gene group score for the Granulocyte traffic group. In some embodiments, this gene group score may be calculated using RNA expression levels of at least three genes (e.g., at least three genes, at least four genes, at least five genes, or at least six genes) in the Granulocyte traffic group, which is defined by its constituent genes: CXCL8, CCR3, CXCR2, CXCL2, CCL11, KITLG, CXCL1, CXCL5, CXCR1.

In some embodiments, an RC TME signature comprises a gene group score for the MDSC group. In some embodiments, this gene group score may be calculated using RNA expression levels of at least three genes (e.g., at least three genes, at least four genes, at least five genes, or at least six genes) in the MDSC group, which is defined by its constituent genes: IDO1, ARG1, IL10, CYBB, PTGS2, IL4I1, IL6.

In some embodiments, an RC TME signature comprises a gene group score for the MDSC traffic signature group. In some embodiments, this gene group score may be calculated using RNA expression levels of at least three genes (e.g., at least three genes, at least four genes, at least five genes, at least six genes, or at least seven genes) in the MDSC traffic group, which is defined by its constituent genes: CCL15, IL6R, CSF2RA, CSF2, CXCL8, CXCL12, IL6, CSF3, CCL26, CXCR4, CXCR2, CSF3R, CSF1, CXCL5, CSF1R.

In some embodiments, an RC TME signature comprises a gene group score for the Macrophage group. In some embodiments, this gene group score may be calculated using RNA expression levels of at least three genes (e.g., at least three genes, at least four genes, at least five genes, at least six genes, or at least seven genes) in the Macrophage group, which is defined by its constituent genes: MRC1, CD163, MSR1, SIGLEC1, IL4I1, CD68, IL10, CSF1R.

In some embodiments, an RC TME signature comprises a gene group score for the Macrophage DC traffic group. In some embodiments, this gene group score may be calculated using RNA expression levels of at least three genes (e.g., at least three genes, at least four genes, at least five genes, at least six genes, or at least seven genes) in the Macrophage DC traffic group, which is defined by its constituent genes: CCL7, CCL2, XCR1, XCL1, CSF1, CCR2, CCL8, CSF1R.

In some embodiments, an RC TME signature comprises a gene group score for the Th2 signature group. In some embodiments, this gene group score may be calculated using RNA expression levels of at least three genes (e.g., at least three genes, at least four genes, at least five genes, at least six genes, or at least seven genes) in the Th2 signature group, which is defined by its constituent genes: IL13, CCR4, IL10, IL5, IL4.

In some embodiments, an RC TME signature comprises a gene group score for the Protumor cytokines group. In some embodiments, this gene group score may be calculated using RNA expression levels of at least three genes (e.g., at least three genes, at least four genes, at least five genes, at least six genes, or at least seven genes) in the Protumor cytokines group, which is defined by its constituent genes: MIF, TGFB1, IL10, TGFB3, IL6, TGFB2, IL22.

In some embodiments, an RC TME signature comprises a gene group score for the Cancer associated fibroblast (CAF) group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than 10 genes) in the Cancer associated fibroblast (CAF) group, which is defined by its constituent genes: PDGFRB, COL6A3, FBLN1, CXCL12, COL6A2, COL6A1, LUM, CD248, COL5A1, MMP2, COL1A1, MFAP5, PDGFRA, LRP1, FGF2, MMP3, FAP, COL1A2, ACTA2.

In some embodiments, an RC TME signature comprises a gene group score for the Matrix group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than 10 genes) in the Matrix group, which is defined by its constituent genes: COL11A1, LAMB3, FN1, COL1A1, COL4A1, ELN, LGALS9, LGALS7, LAMC2, TNC, LAMA3, COL3A1, COL5A1, VTN, COL1A2.

In some embodiments, an RC TME signature comprises a gene group score for the Matrix-remodeling group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than 10 genes) in the Matrix-remodeling group, which is defined by its constituent genes: MMP1, PLOD2, MMP2, MMP12, ADAMTS5, ADAMTS4, LOX, MMP9, MMP11, MMP3, MMP1, CA9.

In some embodiments, an RC TME signature comprises a gene group score for the angiogenesis group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than ten genes) in the angiogenesis group, which is defined by its constituent genes: VEGFA, VEGFB, VEGFC, PDGFC, CXCL8, CXCR2, FLT1, PGF, KDR, ANGPT1, ANGPT2, TEK, VWF, CDH5.

In some embodiments, an RC TME signature comprises a gene group score for the endothelium group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than ten genes) in the endothelium group, which is defined by its constituent genes: NOS3, MMRN1, FLT1, CLEC14A, MMRN2, VCAM1, ENG, VWF, CDH5, KDR.

In some embodiments, an RC TME signature comprises a gene group score for the Proliferation rate group. In some embodiments, this gene group score may be calculated using RNA expression levels of at least three genes (e.g., at least three genes, at least four genes, at least five genes, or at least six genes) in the Proliferation rate group, which is defined by its constituent genes: MKI67, ESCO2, CETN3, CDK2, CCND1, CCNE1, AURKA, AURKB, E2F1, MYBL2, BUB1, CCNB1, MCM2, MCM6.

In some embodiments, an RC TME signature comprises a gene group score for the EMT signature group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than 10 genes) in the EMT signature group, which is defined by its constituent genes: SNAI2, TWIST1, ZEB2, SNAI1, ZEB1, TWIST2, CDH2.

In some embodiments, an RC TME signature comprises a gene group score for the Cyclic Nucleotides Metabolism group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than 10 genes) in the Cyclic Nucleotides Metabolism group, which is defined by its constituent genes: ADCY4, PDE11A, PDE6A, PDE9A, PDE6C, ADCY7, PDE4A, PDE8A, PDE1B, PDE1A, GUCY2C, GUCY1A3, ADCY9, ADCY2, PDE6B, ADCY8, PDE8B, GUCY2F, PDE4C, PDE3A, GUCY1A2, PDE6G, PDE1C, GUCY2D, ADCY10, GUCY1B3, GUCY1B2, PDE7B, PDE5A, PDE6D, NPR2, ADCY5, NPR1, ADCY6, PDE7A, PDE2A, PDE4B, PDE10A, PDE6H, PDE4D, ADCY1, PDE3B, ADCY3.

In some embodiments, an RC TME signature comprises a gene group score for the Glycolysis and Gluconeogenesis group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than 10 genes) in the Glycolysis and Gluconeogenesis group, which is defined by its constituent genes: SLC2A9, PFKL, GCK, PFKFB4, SLC16A7, PCK1, PGAM2, GAPDH, BPGM, G6PC2, FBP2, LDHD, SLC2A3, GPI, ENO1, SLC25A11, PFKFB3, PFKM, LDHAL6B, SLC2A2, G6PC3, SLC2A6, GAPDHS, SLC2A11, PCK2, PFKP, PGK1, ALDOC, SLC2A10, ACYP2, SLC2A4, PKLR, HKDC1, PGK2, SLC2A8, PGAM1, SLC5A1, SLC5A12, SLC16A1, ALDOB, HK3, HK1, SLC5A9, GPD2, PFKFB1, SLC2A7, SLC5A11, SLC5A3, ACYP1, SLC16A8, PFKFB2, ALDOA, SLC5A2, HK2, ENO3, SLC2A12, FBP1, LDHA, LDHB, LDHC, G6PC, SLC2A14, SLC5A8, TPI1, SLC16A3, PKM2, ENO2, PGM1, UEVLD, LDHAL6A, SLC2A1, PGM2; Citric Acid Cycle group: ACLY, FAH, PC, MDH1B, SLC16A7, IREB2, PCK1, MDH1, SLC33A1, ALDH1B1, IDH3B, DLST, PDHB, MDH2, ACO1, IDH1, SLC5A6, HICDH, SLC16A8, GOT1, ME3, ME1, CS, OGDH, SDHA, ALDH5A1, CLYBL, SDHD, IDH3A, SLC25A1, ACSS2, SDHC, ACSS1, SUCLA2, SLC13A5, PDHX, SDHB, ALDH4A1, PCK2, DLD, ACO2, PDHA1, SLC13A2, FAHD1, IDH2, GOT2, ME2, ADSL, SUCLG2, SLC13A3, SUCLG1, SLC25A10, FH, IDH3G, SLC16A1, SLC25A11, PDHA2, DLAT.

In some embodiments, an RC TME signature comprises a gene group score for the Fatty Acid Metabolism group. In some embodiments, this gene group score may be calculated using RNA expression levels of at least three genes (e.g., at least three genes, at least four genes, at least five genes, at least six genes, at least seven genes, at least eight genes, at least nine genes, at least ten genes, or more than 10 genes) in the Fatty Acid Metabolism group, which is defined by its constituent genes: MLYCD, ALDH3A2, SLC27A5, SLC27A3, LIPC, SLC27A2, ACSL4, ACSL1, PCCB, SLC25A20, AADAC, SLC22A4, SLC22A5, ECH1, PCCA, SLC27A1, SLC27A4, CROT, ACSL5, ACSL3, CYP4F12.

In some embodiments, determining an RC TME signature comprises determining a respective gene group score for each of at least two of the following gene groups, using, for a particular gene group, RNA expression levels for at least three genes (e.g., 3, 4, 5, 6, 7, 8, 9, 10, 11, 12, 13, 14, 15, 16 17, 18, 19, 20, all genes in the gene group or any number therebetween) in the particular gene group to determine the gene group score for the particular group, the gene groups including: MHC I group: HLA-A, HLA-B, HLA-C, B2M, TAP1, TAP2, NLRC5, TAPBP; MHC H group: HLA-DQA1, HLA-DMA, HLA-DRB1, HLA-DMB, CIITA, HLA-DPA1, HLA-DPB1, HLA-DRA, HLA-DQB1; Coactivation molecules group: CD28, CD40, TNFRSF4, ICOS, TNFRSF9, CD27, CD80, CD86, CD40LG, CD83, TNFSF4, ICOSLG, TNFSF9, CD70; Effector cells group: IFNG, GZMA, GZMB, PRF1, GZMK, ZAP70, GNLY, FASLG, TBX21, EOMES, CD8A, CD8B; T cell traffic group: CXCL9, CCL3, CXCR3, CXCL10, CXCL11, CCL5, CCL4, CX3CL1, CX3CR1; NK cells group: GZMB, NKG7, CD160, GZMH, CD244, EOMES, KLRK1, NCR1, GNLY, KLRF1, FGFBP2, SH2D1B, KIR2DL4, IFNG, NCR3, KLRC2, CD226; T cells group: TRAC, TRBC2, TBX21, CD3E, CD3D, ITK, TRBC1, CD3G, CD28, TRAT1, CD5; B cells group: CR2, MS4A1, CD79A, FCRL5, STAP1, TNFRSF17, TNFRSF13B, CD19, BLK, CD79B, TNFRSF13C, CD22, PAX5; M1 signature group: IL1B, IL12B, NOS2, SOCS3, IRF5, IL23A, TNF, IL12A, CMKLR1; Th1 signature group: IL12RB2, IL2, TBX21, IFNG, STAT4, IL21, CD40LG; Antitumor cytokines group: IFNA2, CCL3, TNF, TNFSF10, IL21, IFNB1; Checkpoint inhibition group: CTLA4, HAVCR2, CD274, LAG3, BTLA, VSIR, PDCD1LG2, TIGIT, PDCD1; Treg group: TNFRSF18, IKZF2, IL10, IKZF4, CTLA4, FOXP3, CCR8; T reg traffic group: CCL28, CCR10, CCR4, CCR8, CCL17, CCL22, CCL1; Neutrophil signature group: FCGR3B, CD177, CTSG, PGLYRP1, FFAR2, CXCR2, PRTN3, ELANE, MPO, CXCR1; Granulocyte traffic group: CXCL8, CCR3, CXCR2, CXCL2, CCL11, KITLG, CXCL1, CXCL5, CXCR1; MDSC group: IDO1, ARG1, IL10, CYBB, PTGS2, IL4I1, IL6; MDSC traffic group: CCL15, IL6R, CSF2RA, CSF2, CXCL8, CXCL12, IL6, CSF3, CCL26, CXCR4, CXCR2, CSF3R, CSF1, CXCL5, CSF1R; Macrophage group: MRC1, CD163, MSR1, SIGLEC1, IL4I1, CD68, IL10, CSF1R; Macrophage DC traffic group: CCL7, CCL2, XCR1, XCL1, CSF1, CCR2, CCL8, CSF1R; Th2 signature group: IL13, CCR4, IL10, IL5, IL4; Protumor cytokines group: MIF, TGFB1, IL10, TGFB3, IL6, TGFB2, IL22; Cancer associated fibroblast (CAF) group: PDGFRB, COL6A3, FBLN1, CXCL12, COL6A2, COL6A1, LUM, CD248, COL5A1, MMP2, COL1A1, MFAP5, PDGFRA, LRP1, FGF2, MMP3, FAP, COL1A2, ACTA2; Matrix group: COL11A1, LAMB3, FN1, COL1A1, COL4A1, ELN, LGALS9, LGALS7, LAMC2, TNC, LAMA3, COL3A1, COL5A1, VTN, COL1A2; Matrix-remodeling group: MMP1, PLOD2, MMP2, MMP12, ADAMTS5, ADAMTS4, LOX, MMP9, MMP11, MMP3, MMP1, CA9; Angiogenesis group: VEGFA, VEGFB, VEGFC, PDGFC, CXCL8, CXCR2, FLT1, PGF, KDR, ANGPT1, ANGPT2, TEK, VWF, CDH5; endothelium group: NOS3, MMRN1, FLT1, CLEC14A, MMRN2, VCAM1, ENG, VWF, CDH5, KDR; Proliferation rate group: MKI67, ESCO2, CETN3, CDK2, CCND1, CCNE1, AURKA, AURKB, E2F1, MYBL2, BUB1, CCNB1, MCM2, MCM6; EMT signature group: SNAI2, TWIST1, ZEB2, SNAI1, ZEB1, TWIST2, CDH2; Cyclic Nucleotides Metabolism group: ADCY4, PDE11A, PDE6A, PDE9A, PDE6C, ADCY7, PDE4A, PDE8A, PDE1B, PDE1A, GUCY2C, GUCY1A3, ADCY9, ADCY2, PDE6B, ADCY8, PDE8B, GUCY2F, PDE4C, PDE3A, GUCY1A2, PDE6G, PDE1C, GUCY2D, ADCY10, GUCY1B3, GUCY1B2, PDE7B, PDE5A, PDE6D, NPR2, ADCY5, NPR1, ADCY6, PDE7A, PDE2A, PDE4B, PDE10A, PDE6H, PDE4D, ADCY1, PDE3B, ADCY3; Glycolysis and Gluconeogenesis group: SLC2A9, PFKL, GCK, PFKFB4, SLC16A7, PCK1, PGAM2, GAPDH, BPGM, G6PC2, FBP2, LDHD, SLC2A3, GPI, ENO1, SLC25A11, PFKFB3, PFKM, LDHAL6B, SLC2A2, G6PC3, SLC2A6, GAPDHS, SLC2A11, PCK2, PFKP, PGK1, ALDOC, SLC2A10, ACYP2, SLC2A4, PKLR, HKDC1, PGK2, SLC2A8, PGAM1, SLC5A1, SLC5A12, SLC16A1, ALDOB, HK3, HK1, SLC5A9, GPD2, PFKFB1, SLC2A7, SLC5A11, SLC5A3, ACYP1, SLC16A8, PFKFB2, ALDOA, SLC5A2, HK2, ENO3, SLC2A12, FBP1, LDHA, LDHB, LDHC, G6PC, SLC2A14, SLC5A8, TPI1, SLC16A3, PKM2, ENO2, PGM1, UEVLD, LDHAL6A, SLC2A1, PGM2; Citric Acid Cycle group: ACLY, FAH, PC, MDH1B, SLC16A7, IREB2, PCK1, MDH1, SLC33A1, ALDH1B1, IDH3B, DLST, PDHB, MDH2, ACO1, IDH1, SLC5A6, HICDH, SLC16A8, GOT1, ME3, ME1, CS, OGDH, SDHA, ALDH5A1, CLYBL, SDHD, IDH3A, SLC25A1, ACSS2, SDHC, ACSS1, SUCLA2, SLC13A5, PDHX, SDHB, ALDH4A1, PGK2, DLD, ACO2, PDHA1, SLC13A2, FAHD1, IDH2, GOT2, ME2, ADSL, SUCLG2, SLC13A3, SUCLG1, SLC25A10, FH, IDH3G, SLC16A1, SLC25A11, PDHA2, DLAT; and Fatty Acid Metabolism group: MLYCD, ALDH3A2, SLC27A5, SLC27A3, LIPC, SLC27A2, ACSL4, ACSL1, PCCB, SLC25A20, AADAC, SLC22A4, SLC22A5, ECH1, PCCA, SLC27A1, SLC27A4, CROT, ACSL5, ACSL3, CYP4F12.

A list of gene groups is provided in Table 1 below:

TABLE 1
List of Gene Groups, the left column providing the
name of the Gene Group and the right column providing
examples of genes in the Gene Group. In some embodiments,
an RC TME signature may include scores for two or
more of the gene groups in this table.

| Gene Group | Gene Group Genes |
|---|---|
| MHCI | HLA-C, B2M, HLA-B, HLA-A, TAP1, TAP2, |
| | NLRC5, TAPBP |
| MHCII | HLA-DQA1, HLA-DMA, HLA-DRB1, HLA-DMB, |
| | CIITA, HLA-DPA1, HLA-DPB1, HLA-DRA, |
| | HLA-DQB1 |
| Coactivation | CD80, TNFRSF4, CD27, CD83, TNFSF9, |
| molecules | CD40LG, CD70, ICOS, CD86, CD40, TNFSF4, |
| | ICOSLG, TNFRSF9, CD28 |
| Effector | PRF1, GZMB, TBX21, CD8B, ZAP70, IFNG, |
| cells | GZMK, EOMES, FASLG, CD8A, GZMA, GNLY |
| T cell | CXCL9, CCL3, CXCR3, CXCL10, CXCL11, |
| traffic | CCL5, CCL4, CX3CL1, CX3CR1 |
| NK cells | GZMB, NKG7, CD160, GZMH, CD244, EOMES, |
| | KLRK1, NCR1, GNLY, KLRF1, FGFBP2, SH2D1B, |
| | KIR2DL4, IFNG, NCR3, KLRC2, CD226 |
| T cells | TRAC, TRBC2, TBX21, CD3E, CD3D, ITK, TRBC1, |
| | CD3G, CD28, TRAT1, CD5 |
| B cells | CR2, MS4A1, CD79A, FCRL5, STAP1, TNFRSF17, |
| | TNFRSF13B, CD19, BLK, CD79B, TNFRSF13C, |
| | CD22, PAX5 |
| M1 | IL1B, IL12B, NOS2, SOCS3, IRF5, IL23A, TNF, |
| signatures | IL12A, CMKLR1 |
| Th1 | IL12RB2, IL2, TBX21, IFNG, STAT4, IL21, |
| signature | CD40LG |
| Antitumor | IFNA2, CCL3, TNF, TNFSF10, IL21, IFNB1 |
| cytokines | |
| Checkpoint | CTLA4, HAVCR2, CD274, LAG3, BTLA, VSIR, |
| inhibition | PDCD1LG2, TIGIT, PDCD1 |
| Treg | TNFRSF18, IKZF2, IL10, IKZF4, CTLA4, FOXP3, |
| | CCR8 |
| T reg traffic | CCL28, CCR10, CCR4, CCR8, CCL17, CCL22, CCL1 |
| Neutrophil | FCGR3B, CD177, CTSG, PGLYRP1, FFAR2, CXCR2, |
| signature | PRTN3, ELANE, MPO, CXCR1 |
| Granulocyte | CXCL8, CCR3, CXCR2, CXCL2, CCL11, KITLG, |
| traffic | CXCL1, CXCL5, CXCR1 |
| MDSC | ARG1, IL4I1, IL10, CYBB, IL6, PTGS2, IDO1 |
| MDSC | CCL15, IL6R, CSF2RA, CSF2, CXCL8, CXCL12, |
| traffic | IL6, CSF3, CCL26, CXCR4, CXCR2, CSF3R, |
| | CSF1, CXCL5, CSF1R |
| Macrophages | MRC1, CD163, MSR1, SIGLEC1, IL4I1, CD68, |
| | IL10, CSF1R |
| Macrophage | CCL7, CCL2, XCR1, XCL1, CSF1, CCR2, CCL8, |
| DC | CSF1R |
| traffic | |
| Th2 | IL13, CCR4, IL10, IL5, IL4 |
| signature | |
| Protumor | MIF, TGFB1, IL10, TGFB3, IL6, TGFB2, IL22 |
| cytokines | |
| CAF | PDGFRB, COL6A3, FBLN1, CXCL12, COL6A2, |
| | COL6A1, LUM, CD248, COL5A1, MMP2, COL1A1, |
| | MFAP5, PDGFRA, LRP1, FGF2, MMP3, FAP, |
| | COL1A2, ACTA2 |
| Matrix | COL11A1, LAMB3, FN1, COL1A1, COL4A1, ELN, |
| | LGALS9, LGALS7, LAMC2, TNC, LAMA3, COL3A1, |
| | COL5A1, VTN, COL1A2 |
| Matrix | MMP1, PLOD2, MMP2, MMP12, ADAMTS5, |
| remodeling | ADAMTS4, LOX, MMP9, MMP11, MMP3, MMP7, |
| | CA9 |
| Angiogenesis | PGF, CXCL8, FLT1, ANGPT1, ANGPT2, VEGFC, |
| | VEGFB, CXCR2, VEGFA, VWF, CDH5, CXCL5, |
| | PDGFC, KDR, TEK |
| Endothelium | NOS3, MMRN1, FLT1, CLEC14A, MMRN2, VCAM1, |
| | ENG, VWF, CDH5, KDR |
| Proliferation | AURKA, MCM2, CCNB1, MYBL2, MCM6, CDK2, |
| rate | E2F1, CCNE1, ESCO2, CCND1, AURKB, BUB1 |
| | MKI67, PLK1, CETN3 |
| EMT | SNAI2, TWIST1, ZEB2, SNAI1, ZEB1, TW1ST2, |
| signature | CDH2 |
| Cyclic | ADCY4, PDE11A, PDE6A, PDE9A, PDE6C, ADCY7, |
| Nucleotides | PDE4A, PDE8A, PDE1B, PDE1A, GUCY2C, |
| Metabolism | GUCY1A3, ADCY9, ADCY2, PDE6B, ADCY8, |
| | PDE8B, GUCY2F, PDE4C, PDE3A, GUCY1A2, |
| | PDE6G, PDE1C, GUCY2D, ADCY10, GUCY1B3, |
| | GUCY1B2, PDE7B, PDE5A, PDE6D, NPR2, ADCY5, |
| | NPR1, ADCY6, PDE7A, PDE2A, PDE4B, PDE10A, |
| | PDE6H, PDE4D, ADCY1, PDE3B, ADCY3 |
| Glycolysis and | SLC2A9, PFKL, GCK, PFKFB4, SLC16A7, PCK1, |
| Gluconeo- | PGAM2, GAPDH, BPGM, G6PC2, FBP2, LDHD, |
| genesis | SLC2A3, GPI, ENO1, SLC25A11, PFKFB3, PFKM, |
| | LDHAL6B, SLC2A2, G6PC3, SLC2A6, GAPDHS, |
| | SLC2A11, PCK2, PFKP, PGK1, ALDOC, SLC2A10, |
| | ACYP2, SLC2A4, PKLR, HKDC1, PGK2, SLC2A8, |
| | PGAM1, SLC5A1, SLC5A12, SLC16A1, ALDOB, |
| | HK3, HK1, SLC5A9, GPD2, PFKFB1, SLC2A7, |
| | SLC5A11, SLC5A3, ACYP1, SLC16A8, PFKFB2, |
| | ALDOA, SLC5A2, HK2, ENO3, SLC2A12, FBP1, |
| | LDHA, LDHB, LDHC, G6PC, SLC2A14, SLC5A8, |
| | TPI1, SLC16A3, PKM2, ENO2, PGM1, UEVLD, |
| | LDHAL6A, SLC2A1, PGM2 |
| Citric Acid | ACLY, FAH, PC, MDH1B, SLC16A7, IREB2, PCK1, |
| Cycle | MDH1, SLC33A1, ALDH1B1, IDH3B, DLST, PDHB, |
| | MDH2, ACO1, IDH1, SLC5A6, HICDH, SLC16A8, |
| | GOT1, ME3, ME1, CS, OGDH, SDHA, ALDH5A1, |
| | CLYBL, SDHD, IDH3A, SLC25A1, ACSS2, SDHC, |
| | ACSS1, SUCLA2, SLC13A5, PDHX, SDHB, |
| | ALDH4A1, PCK2, DLD, ACO2, PDHA1, SLC13A2, |
| | FAHD1, IDH2, GOT2, ME2, ADSL, SUCLG2, |
| | SLC13A3, SUCLG1, SLC25A10, FH, IDH3G, |
| | SLC16A1, SLC25A11, PDHA2, DLAT |
| Fatty Acid | MLYCD, ALDH3A2, SLC27A5, SLC27A3, UPC, |
| Metabolism | SLC27A2, ACSL4, ACSL1, PCCB, SLC25A20, |
| | AADAC, SLC22A4, SLC22A5, ECH1, PCCA, |
| | SLC27A1, SLC27A4, CROT, ACSL5, ACSL3, |
| | CYP4F12 |
| ECM | ADAMS, ADAMTS4, C1QL3, CST7, CTSW, CXCL8, |
| Associated | FASLG, LTB, MUC1, OSM, P4HA2, SCUBE1, |
| | SEMA4B, SEMA7A, SERPINE1, TCHH, TGFA, |
| | TGM2, TNFSF11, TNFSF9, WNT10B |
| TLS Kidney | ZNF683, POU2AF1, LAX1, CD79A, CXCL9, XCL2, |
| | JCHAIN, SLAMF7, CD38, SLAMF1, TNFRSF17, |
| | IRF4, HSH2D, PLA2G2D, MZB1 |
| NRF2 | TRIM16L, UGDH, KIAA1549, PANX2, FECH, LRP8, |
| signature | AKR1C2, FTH1, AKR1C3, CBR1, PFN2, CBX2, TXN, |
| | CYP4F11, CYP4F3, AKR1C1, AKR1B15, G6PD, |
| | PRDX1, TALDO1, EPT1, SRXN1, JAKMIP3, FTHL3, |
| | UCHL1, TXNRD1, C1orf131, CASKIN1, PGD, GPX2, |
| | OSGIN1, KIAA0319, CABYR, AIFM2, TRIM16, |
| | AKR1B10, GCLC, ABCC2, ETFB, IDH1, MAFG, |
| | NECAB2, ME1, PTGR1, PIR, GSR, RIT1, GCLM, |
| | ALDH3A1, NQO1, PKD1L2, NRG4, ABHD4, HRG, |
| | SLC7A11 |
| tRCC | FST, TRIM63, SLC10A2, ANTXRL, ERVV-2, SNX22, |
| signature | INHBE, SV2B, FAM124A, EPHA5, LUZP2, CPEB1, |
| | HOXB13, ALLC, KCNF1, NDRG4, GREB1, ASTN1, |
| | JSRP1, UBE2U, KCNQ4, MYO7B, BRINP2, C1QL2, |
| | CCDC136, SLC51B, CATSPERG, PMEL, BIRC7, |
| | PLK5, ADARB2, CFAP61, TUBB4A, PLIN4, ABCB5, |
| | SYT3, HCN4, CTSK, SPACA1, TRIM67, NMRK2, |
| | LGI3, ARHGEF4, NTSR2, KEL, SNCB, PLD5, |
| | ADGRB1, CYP17A1, IGFBPL1, TRIM71, SLC45A2, |
| | TP73, IP6K3, HABP2, RGS20, IGFN1, CDH17 |

As described above, aspects of the disclosure relate to determining an RC TME signature for a subject. That signature may include a gene group scores (e.g., gene group scores generated using RNA expression data for some or all of the gene groups listed in Table 1). Aspects of determining of these signatures is described next with reference to FIG. 3.

In some embodiments, an RC TME signature comprises gene group scores generated using a gene set enrichment analysis (GSEA) technique to determine a gene group score for one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37) gene groups listed in Table 1. In some embodiments, each gene group score is generated using a RNA levels for at least some of the genes in each gene group. In some embodiments, using a GSEA technique comprises using single-sample GSEA. Aspects of single sample GSEA (ssGSEA) are described in Barbie et al. Nature. 2009 Nov. 5; 462(7269): 108-112, the entire contents of which are incorporated by reference herein. In some embodiments, ssGSEA is performed according to the following formula:

$$ssGSEA score = \frac{{\sum }_{i}^{N} {r}_{t}^{1.25}}{{\sum }_{i}^{N} {r}_{t}^{0.25}} - \frac{( M - N + 1 )}{2}$$

where ri represents the rank of the ith gene in expression matrix, where N represents the number of genes in the gene set (e.g., the number of genes in the first gene group when ssGSEA is being used to determine a score for the first gene group using expression levels of the genes in the first gene group), and where M represents total number of genes in expression matrix. Additional, suitable techniques of performing GSEA are known in the art and are contemplated for use in the methods described herein without limitation. In some embodiments, an RC TME signature is calculated by performing ssGSEA on expression data from a plurality of subjects, for example expression data from one or more cohorts of subjects, such as: KIRC, JAVELIN101, Immotion150, Immotion151, ICGC RECA EU, PUB_KIRC_CPTAC3, PUB_RCC_VanAllen_phs001493, WU_ccRCC_RCCTC, PUB_ccRCC_Chinese_2020, ccRCC_CheckMates_2020, PUB_ccRCC_Sato_2013, COMPARZ, E-MTAB-3267, GSE2109, GSE53757, GSE73731, and GSE40435, in order to produce a plurality of gene group scores.

FIG. 3 depicts an illustrative process 108 for determining gene enrichment score, according to some embodiments of the technology as described herein. As shown in the example of FIG. 3, a “RC TME signature” comprises multiple gene group scores 320 determined for respective multiple gene groups. Each gene group score, for a particular gene group, is computed by performing GSEA 310 (e.g., using ssGSEA) on RNA expression data for one or more (e.g., at least two, at least three, at least four, at least five, at least six, etc., or all) genes in the particular gene group 300.

For example, as shown in FIG. 3, a gene group score (labelled “Gene Group Score 1”) for gene group 1 (e.g., the Treg cells group) is computed from RNA expression data for one or more genes in gene group 1. As another example, a gene group score (labelled “Gene Group Score 2”) for gene group 2 (e.g., the Th1 group) is computed from RNA expression data for one or more genes in gene group 2. As another example, a gene group score (labelled “Gene Group Score 3”) for gene group 3 (e.g., the MHC II group) is computed from RNA expression data for one or more genes in gene group 3. As another example, a gene group score (labelled “Gene Group Score 4”) for gene group 4 (e.g., the Effector cells group) is computed from RNA expression data for one or more genes in gene group 4. As another example, a gene group score (labelled “Gene Group Score 5”) for gene group 5 (e.g., the Antitumor cytokines group) is computed from RNA expression data for one or more genes in gene group 5. As another example, a gene group score (labelled “Gene Group Score 6”) for gene group 6 (e.g., the M1 group) is computed from RNA expression data for one or more genes in gene group 6. As another example, a gene group score (labelled “Gene Group Score 7”) for gene group 7 (e.g., the Neutrophil signature group) is computed from RNA expression data for one or more genes in gene group 7. As another example, a gene group score (labelled “Gene Group Score 8”) for gene group 8 (e.g., the Checkpoint inhibition group) is computed from RNA expression data for one or more genes in gene group 8.

Although the example of FIG. 3 shows that the gene expression group expression score includes eight gene group scores for a respective set of eight gene groups, it should be appreciated that in other embodiments, the first gene expression signature may include scores for any suitable number of groups (e.g., not just 8; the number of groups could be fewer or greater than 8). As indicated by the vertical ellipsis in FIG. 3, determining gene group scores of RC TME signature may comprise determining gene group scores for 9, 10, 11, 12, 13, 14, 15, 16, 17, 18, 19, 20, 21, 22, 23, 24, 25, 26, 27, 28, 29, 30, 31, 32, 33, 34, 35, 36, 37, or more gene groups using RNA expression data from one or more respective genes in each respective gene group, as aspects of the technology described herein are not limited in this respect. In another example, an RC TME signature may include scores for only a subset of the gene groups listed in Table 1 above.

The number of genes in a gene group used to determine a gene group expression score may vary. In some embodiments, all RNA expression levels for all genes in a particular gene group may be used to determine a gene group score for the particular gene group. In other embodiments, RNA expression data for fewer than all genes may be used (e.g., RNA expression levels for at least two genes, at least three genes, at least five genes, between 2 and 10 genes, between 5 and 15 genes, or any other suitable range within these ranges).

In some embodiments, RNA expression levels for a particular gene group may be embodied in at least one data structure having fields storing the expression levels. The data structure or data structures may be provided as input to software comprising code that implements a GSEA technique (e.g., the ssGSEA technique) and processes the expression levels in the at least one data structure to compute a score for the particular gene group. In some embodiments, ssGSEA is performed on expression data comprising three or more (e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37) gene groups set forth in Table 1. In some embodiments, each of the gene groups separately comprises one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75 or more) genes listed in Table 1. In some embodiments, an RC TME signature is produced by performing ssGSEA on 33 of the gene groups in Table 1, each gene group including all listed genes in Table 1. In some embodiments, an RC TME signature is produced by performing ssGSEA on 37 of the gene groups in Table 1, each gene group including all listed genes in Table 1

In some embodiments, one or more (e.g., a plurality) of enrichment scores are normalized in order to produce s RC TME signature for the expression data (e.g., expression data of the subject or of a cohort of subjects). In some embodiments, the enrichment scores are normalized by median scaling. In some embodiments, median scaling produces an RC TME signature of the subject. In some embodiments, median scaling comprises clipping the range of enrichment scores, for example clipping to about −1.0 to about +1.0, −2.0 to about +3.0, −3.0 to about +3.0, −4.0 to +4.0, −5.0 to about +5.0.

In some embodiments, an RC TME signature of a subject processed using a clustering algorithm to identify an RC tumor microenvironment type (e.g. an RC TME type). In some embodiments, the clustering comprises unsupervised clustering. In some embodiments, the unsupervised clustering comprises a dense clustering approach. In some embodiments, an RC TME signature of a subject is compared to pre-existing clusters of RC TME types and assigned an RC TME type based on that comparison. In some embodiments, clustering comprises generating a graph with samples at nodes and correlation of the ssGSEA scores at edges. In some embodiments, each node has 75 neighbors. In some embodiments, clustering further comprises applying the Leiden algorithm to the resulting graph.

In some embodiments, a RC TME signature of a subject is compared to pre-existing clusters of RC TME types and assigned a RC TME type based on that comparison.

Some aspects of determining gene group scores for gene groups are also described in U.S. Patent Publication No. 2020-0273543, entitled “SYSTEMS AND METHODS FOR GENERATING, VISUALIZING AND CLASSIFYING MOLECULAR FUNCTIONAL PROFILES”, the entire contents of which are incorporated by reference herein.

### Updating RC TME Clusters Based on New Data

Techniques for generating RC TME clusters are described herein. It should be appreciated that the RC TME clusters may be updated as additional RC TME signatures are computed for patients. In some embodiments, the RC TME signature of the subject is one of a threshold number RC TME signatures for a threshold number of subjects. In some embodiments, when the threshold number of RC TME signatures is generated the RC TME signature clusters are updated. For example, once a threshold number of new RC TME signatures are obtained (e.g., 1 new signature, 10 new signatures, 100 new signatures, 500 new signatures, any suitable threshold number of signatures in the range of 10-1,000 signatures), the new signatures may be combined with the RC TME signatures previously used to generate the RC TME clusters and the combined set of old and new RC TME signatures may be clustered again (e.g., using any of the clustering algorithms described herein or any other suitable clustering algorithm) to obtain an updated set of RC TME signature clusters.

In this way, data obtained from a future patient may be analyzed in a way that takes advantage of information learned from patients whose RC TME signature was computed prior to that of the future patient. In this sense, the machine learning techniques described herein (e.g., the unsupervised clustering machine learning techniques) are adaptive and learn with the accumulation of new patient data. This facilitates improved characterization of the RC TME type that future patients may have and may improve the selection of treatment for those patients.

### Myogenesis Signature

Aspects of the disclosure relate to methods for generating a myogenesis signature for a subject. The disclosure is based, in part, on the recognition that a myogenesis signature calculated as described herein can be used, in some embodiments, to identify subjects that have an increased likelihood of being a non-responder to treatment with immuno-oncology (IO) agents. In some embodiments, a myogenesis signature is generated using RNA expression data for at least some (e.g., 2, 3, 4, 5, 6, 7, 8, 9, 10, 11, 12, 13, or 14) of the genes listed in Table 2.

In some embodiments, a myogenesis signature comprises a myogenesis gene group score. In some embodiments, a myogenesis gene group score is generated using RNA expression levels of at least some (e.g., 2, 3, 4, 5, 6, 7, 8, 9, 10, 11, 12, 13, or 14) of the genes listed in Table 2). In some embodiments, generating the myogenesis gene group score comprises performing GSEA (e.g., ssGSEA) using RNA expression data for two or more genes listed in Table 2. In some embodiments, median scaling is performed on the gene expression (e.g., gene enrichment) scores resulting from the GSEA. A myogenesis signature may be expressed as a score ranging from −3 to 20 (e.g., −3, −2, −1, 0, 1, 2, 3, 4, 5, 6, 7, 8, 9, 10, 11, 12, 13, 14, 15, 16, 17, 18, 19, or 20). In some embodiments, a subject having a myogenesis signature (score) higher than 4 is considered as having a “high myogenesis score”. In some embodiments, a subject having a myogenesis signature (score) higher than 8 is considered as having a “high myogenesis score”. In some embodiments, a subject having a myogenesis signature (score) higher than 10 is considered as having a “high myogenesis score”. In some embodiments, a subject having a myogenesis signature (score) higher than 15 is considered as having a “high myogenesis score”. In some embodiments, a subject having a “high” myogenesis signature (score) is considered to be a “non-responder to IO therapy”. A “non-responder to IO therapy” is a subject having RC (e.g., ccRCC) that is significantly less likely to respond to treatment with an immuno-therapeutic (IO) agent relative to a subject (e.g., an RC subject) not having a “high” myogenesis signature (score).

The number of genes in the gene group used to determine the myogenesis signature may vary. In some embodiments, all RNA expression levels for all genes in a myogenesis signature gene group may be used to determine the myogenesis gene group score. In other embodiments, RNA expression data for fewer than all genes may be used (e.g., RNA expression levels for 2, 3, 4, 5, 6, 7, 8, 9, 10, 11, 12, or 13 genes). A list of genes in the Myogenesis Signature gene group is provided in Table 2 below:

TABLE 2
List of Myogenesis Signature Gene Groups, the left column
providing the name of the Gene Group and the right column
providing examples of genes in the Gene Group

| Gene Group | Gene Group Genes |
|---|---|
| Myogenesis signature | CASQ1 |
| | TNNI1 |
| | MB |
| | MYLPF |
| | MYH7 |
| | CKM |
| | MYL2 |
| | MYL1 |
| | CSRP3 |
| | ACTA1 |
| | MYOZ1 |
| | TNNT3 |
| | TNNC2 |
| | TNNC1 |

### Generating RC TME Signature and Identifying TME Type

As described herein, FIG. 1 illustrates the determination of a subject's RC TME signature and, optionally, identification of the subject's prognosis using the identified RC TME signature.

As described herein, in some embodiments, one of a plurality of different RC TME types may be identified for the subject using the RC TME signature determined for the subject using the techniques described herein. In some embodiments, the RC TME type comprises RC TME type A, RC TME type B, RC TME type C, RC TME type D, and RC TME type E.

In some embodiments, each of the plurality of RC TME types is associated with a respective RC TME signature cluster in a plurality of RC TME signature clusters. The RC TME type for a subject may be determined by: (1) associating the RC TME signature of the subject with a particular one of the plurality of RC TME signature clusters; and (2) identifying the RC TME type for the subject as the RC TME type corresponding to the particular one of the plurality of RC TME signature clusters to which the RC TME signature of the subject is associated.

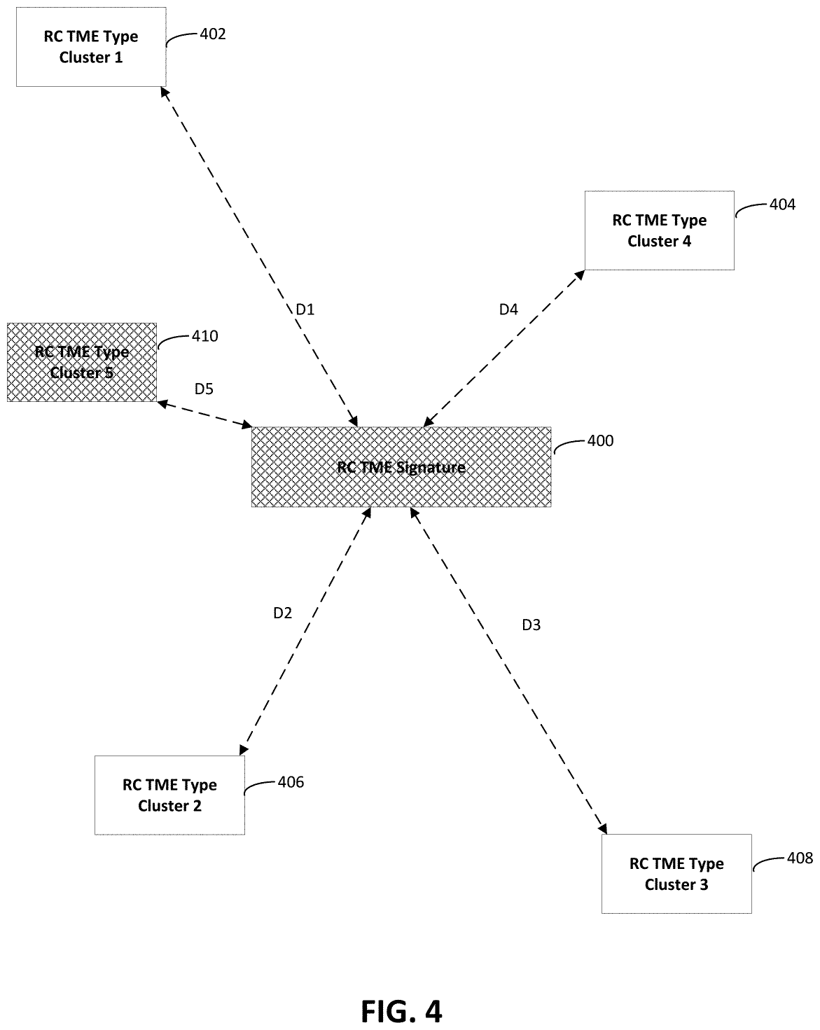

FIG. 4 shows an illustrative RC TME signature 400. In some embodiments, the RC TME signature comprises at least three gene group scores for the gene groups listed in Table 1. However, it should be appreciated, that an RC TME signature may include more or fewer scores than the number of scores shown in FIG. 4 (e.g., by omitting scores for one or more of the gene groups listed in Table 1 or by including scores for one or more other gene groups in addition to or instead of the gene groups listed in Table 1). In some embodiments, an RC TME signature may be embodied in at least one data structure comprising fields storing the gene group scores part of the RC TME signature.

In some embodiments, the RC TME signature clusters may be generated by: (1) obtaining RC TME signatures (using the techniques described herein) for a plurality of subjects; and (2) clustering the RC TME signatures so obtained into the plurality of clusters. Any suitable clustering technique may be used for this purpose including, but not limited to, a dense clustering algorithm, spectral clustering algorithm, k-means clustering algorithm, hierarchical clustering algorithm, and/or an agglomerative clustering algorithm.

For example, inter-sample similarity may be calculated using a Pearson correlation. A distance matrix may be converted into a graph where each sample forms a node and two nodes form an edge with a weight equal to their Pearson correlation coefficient. Edges with weight lower than a specified threshold may be removed. A Louvain community detection algorithm may be applied to calculate graph partitioning into clusters. To mathematically determine the optimum weight threshold for observed clusters minimum Davies Bouldin, maximum Calinski-Harabasz, and Silhouette techniques may be employed. Separations with low-populated clusters (<5% of samples) may be excluded.

Accordingly, in some embodiments, generating the RC TME signature clusters involves: (A) obtaining multiple sets of RNA expression data obtained by sequencing biological samples from multiple respective subjects, each of the multiple sets of RNA expression data indicating RNA expression levels for genes in a first plurality of gene groups (e.g., one or more of the gene groups in Table 1); (B) generating multiple RC TME signatures from the multiple sets of RNA expression data, each of the multiple RC TME signatures comprising gene group scores for respective gene groups, the generating comprising, for each particular one of the multiple RC TME signatures: (i) determining the RC TME signature by determining the gene group scores using the RNA expression levels in the particular set of RNA expression data for which the particular one RC TME signature is being generated, and (ii) clustering the multiple RC signatures to obtain the plurality of RC TME signature clusters.

The resulting RC TME signature clusters may each contain any suitable number of RC TME signatures (e.g., at least 10, at least 100, at least 500, at least 500, at least 1000, at least 5000, between 100 and 10,000, between 500 and 20,000, or any other suitable range within these ranges), as aspects of the technology described herein are not limited in this respect. The number of RC TME signature clusters in this example is five. And although, in some embodiments, it may be possible that the number of clusters is different, it should be appreciated that an important aspect of the present disclosure is the inventors' discovery that RC may be characterized into five types based upon the generation of RC TME signatures using methods described herein.

For example, as shown in FIG. 4, a subject's RC TME signature 400 may be associated with one of five RC TME clusters: 402, 404, 406, 408, and 410. Each of the clusters 402, 404, 406, 408 and 410 may be associated with respective RC TME type. In this example, the RC TME signature 400 is compared to each cluster (e.g., using a distance-based comparison or any other suitable metric) and, based on the result of the comparison, the RC TME signature 400 is associated with the closest RC signature cluster (when a distance-based comparison is performed, or the “closest” in the sense of whatever metric or measure of distance is used). In this example, RC TME signature 400 is associated with RC TME Type Cluster 5 410 (as shown by the consistent shading) because the measure of distance D5 between the RC TME signature 400 and (e.g., a centroid or other point representative of) cluster 410 is smaller than the measures of the distance D1, D2, D3, and D4 between the RC TME signature 400 and (e.g., a centroid or other point(s) representative of) clusters 402, 404, 406, and 408, respectively.

In some embodiments, a subject's RC TME signature may be associated with one of five RC TME signature clusters by using a machine learning technique (e.g., such as k-nearest neighbors (KNN) or any other suitable classifier) to assign the RC TME signature to one of the five RC TME signature clusters. The machine learning technique may be trained to assign RC TME signatures on the meta-cohorts represented by the signatures in the clusters.

In some embodiments, RC TME types include RC TME type A, RC TME type B, RC TME type C, RC TME type D, and RC TME type E. The RC TME types described herein may be described by qualitative characteristics, for example high signals for certain gene expression signatures or scores or low signals for certain other gene expression signatures or scores. In some embodiments, a “high” signal refers to a gene expression signal or score (e.g., an enrichment score) that is at least 1-fold, 2-fold, 3-fold, 4-fold, 5-fold, 6-fold, 7-fold, 8-fold, 9-fold, 10-fold, 20-fold, 50-fold, 100-fold, 1000-fold, or more increased relative to the score of the same gene or gene group in a subject having a different type of RC. In some embodiments, a “low” signal refers to a gene expression signal or score (e.g., an enrichment score) that is at least 1-fold, 2-fold, 3-fold, 4-fold, 5-fold, 6-fold, 7-fold, 8-fold, 9-fold, 10-fold, 20-fold, 50-fold, 100-fold, 1000-fold, or more decreased relative to the score of the same gene or gene group in a subject having a different type of RC TME.

In some embodiments, a subject is identified as having “Immune-enriched, fibrotic (IE/F)”, also referred to as “RC TME type A” RC. In some embodiments, RC TME type A is characterized by a high prevalence of immune cells and a high percentage of cancer-associated fibroblasts (CAF) relative to other RC TME types. In some embodiments, RC TME type A comprises abundant pro-tumor immune-suppressive infiltrate, including a significant number of regulatory T cells. In some embodiments, the percentage of malignant cells in RC TME type A is low relative to other RC TME types. In some embodiments, mutations in tumor suppressor BAP1 are frequent in RC TME type A. In some embodiments, subjects having RC TME type A are responsive to immune checkpoint inhibitors, alone or in combination with tyrosine kinase inhibitors. In some embodiments RC TME type A is characterized by a high tumor proliferation rate relative to other RC TME types. In some embodiments, subjects having RC TME type A have a poor prognosis relative to subjects having other RC TME types.

In some embodiments, a subject is characterized as having “Immune-enriched, non-fibrotic (IE)”, also referred to as “RC TME type B”. In some embodiments, RC TME type B is characterized by abundant immune-active infiltrate including cytotoxic effector cells, and low prevalence of stromal and fibrotic elements relative to other RC TME types. In some embodiments, RC TME type B is characterized by immune-induced inflammation. In some embodiments, subjects having RC TME type B comprise mutations in tumor suppressor BAP1. In some embodiments, subjects having RC TME type B are responsive to immune checkpoint inhibitors, alone or in combination with tyrosine kinase inhibitors.

In some embodiments, a subject is characterized as having “Fibrotic (F)” also referred to as “RC TME type C”. In some embodiments, RC TME type C is highly fibrotic (relative to other RC TME types), with dense collagen formation. In some embodiments, RC TME type C is characterized as having less inflammation than certain other RC TME types. In some embodiments, RC TME type C is characterized by minimal leukocyte/lymphocyte infiltration relative to other RC TME types. In some embodiments, Cancer-associated fibroblasts (CAF) are abundant in type C RC. In some embodiments, signs of epithelial-mesenchymal transition (EMT) are present in subjects having RC TME type C. In some embodiments, RC TME type C is associated with poor prognosis relative to other RC TME types.

In some embodiments, a subject is characterized as having “Immune desert with metabolic content (D)”, also referred to as “RC TME type D”. In some embodiments, the RC TME D type contains the highest malignant cell percentage relative to other RC TME types, and is characterized by minimal or complete absence of leukocyte/lymphocyte infiltration. In some embodiments, immune-mediated inflammation is not present. In some embodiments, signs of metabolic activation are present in subjects having RC TME type D. In some embodiments, RC TME type D is associated with a good prognosis relative to other RC TME types.

In some embodiments, a subject is characterized as having “Angiogenic, non-inflamed”, also referred to as “RC TME type E”. In some embodiments, RC TME type E is characterized by intense angiogenesis and low levels of immune infiltrate relative to other RC TME types. In some embodiments, signs of epithelial-mesenchymal transition (EMT) are present in subjects having RC TME type E. In some embodiments, RC TME type E is associated with low cancer stages and usually does not need to be treated. In some embodiments, subjects having RC TME type E are often responsive to tyrosine kinase inhibitors (TKIs). In some embodiments, RC TME type E is associated with good prognosis relative to other RC TME types.

In some embodiments, the present disclosure provides methods for identifying an RC subject's prognosis using an RC TME signature generated using methods described herein.

In some embodiments, the methods comprise identifying the subject as having a decreased risk of RC progression relative to other RC TME types when the subject is assigned RC TME type E or RC TME D. In some embodiments, “decreased risk of RC progression” may indicate better prognosis of RC or decreased likelihood of having advanced disease in a subject. In some embodiments, “decreased risk of RC progression” may indicate that the subject who has RC is expected to be more responsive to certain treatments. For instance, “decreased risk of RC progression” indicates that a subject is at least 10%, 20%, 30%, 40%, 50%, 60%, 70%, 80%, 90%, or 100% likely to experience a progression-free survival event (e.g., relapse, retreatment, or death) than another RC patient or population of RC patients (e.g., patients having RC, but not the same RC TME type as the subject).

In some embodiments, the methods further comprise identifying the subject as having an increased risk of RC progression relative to other RC TME types when the subject is assigned a RC TME type other than RC TME type E, for example RC TME type A. In some embodiments, “increased risk of RC progression” may indicate less positive prognosis of RC or increased likelihood of having advanced disease in a subject. In some embodiments, “increased risk of RC progression” may indicate that the subject who has RC is expected to be less responsive or unresponsive to certain treatments and show less or no improvements of disease symptoms. For instance, “increased risk of RC progression” indicates that a subject is at least 10%, 20%, 30%, 40%, 50%, 60%, 70%, 80%, 90%, or 100% more likely to experience a progression-free survival event (e.g., relapse, retreatment, or death) than another RC patient or population of RC patients (e.g., patients having RC, but not the same RC TME type as the subject).

### Responder Scores

Aspects of the disclosure relate to methods for determining whether or not a subject having RC (e.g., ccRCC) is likely to respond to certain therapeutic agents, such as immune-therapeutic (IO) agents or TKIs.

In some embodiments, the therapeutic agents are immuno-oncology (IO) agents. An IO agent may be a small molecule, peptide, protein (e.g., antibody, such as monoclonal antibody), interfering nucleic acid, or a combination of any of the foregoing. In some embodiments, the IO agents comprise a PD1 inhibitor, PD-L1 inhibitor, or PD-L2 inhibitor. Examples of IO agents include but are not limited to cemiplimab, nivolumab, pembrolizumab, avelumab, durvalumab, atezolizumab, BMS1166, BMS202, ipilimumab, etc.

In some embodiments, the therapeutic agents are tyrosine kinase inhibitors (TKIs). A TKI may be a small molecule, peptide, protein (e.g., antibody, such as monoclonal antibody), interfering nucleic acid, or a combination of any of the foregoing. Examples of TKIs include but are not limited to Imatinib mesylate (Gleevec®), Dasatinib (Sprycel®), Nilotinib (Tasigna®), Bosutinib (Bosulif®), Sunitinib (Sutent®), etc.

Aspects of the disclosure relate to methods for determining the likelihood of a subject having RC (e.g., ccRCC) responding to an IO agent. The disclosure is based, in part, on the identification of certain subgroups of RC patients that comprise biomarkers indicative of their response to IO agents. In some embodiments, when it is determined (e.g., a subject is identified as having, using methods described herein) that the subject comprises one or more of the following biomarkers, that subject is unlikely to respond to IO therapy: high Ploidy (e.g., as calculated by RTumor bioinformatics analysis), a high Myogenic Signature (e.g., as described throughout the specification for example in the section entitled Myogenesis Signature), RC TME type E (as determined by methods described throughout the specification), presence of mTOR activating mutations, or presence of mutations in antigen presentation machinery. Examples of mTOR activating mutations include but are not limited to mutations in MTOR, mutations in TSC1/2, mutations in PTEN, and mutations in MET, and those described in Cancer Discov. 2014 May; 4(5):554-63. Doi: 10.1158/2159-8290.CD-13-0929. Epub 2014 Mar. 14. Examples of mutations in antigen presentation machinery include but are not limited to mutations in PSMB5, PSMB6, PSMB7, PSMB8, PSMB9, PSMB10, TAP1, TAP2, ERAP1, ERAP2, CANX, CALR, PDIA3, TAPBP, B2M, HLA-A, HLA-B, and HLA-C. In some embodiments, a subject having one or more of the aforementioned biomarkers is referred to as an “IO non-responder”.

In some aspects, the disclosure provides a method for predicting the likelihood of a subject responding to an immuno-oncology (IO) agent by identifying an RC TME type for the subject using gene expression data for the subject, and then using a machine learning model to obtain a responder score which is indicative of the subject's likelihood of responding to IO. In some embodiments, the machine learning model comprises a gradient boosting model. In some embodiments, a machine learning model comprises a CatBoost classifier. In some embodiments, the machine learning model is trained using the following inputs from a plurality of samples (e.g., samples derived from a cohort of patients): RC TME type; expression level of PD1, PD-L1, and/or PD-L2 obtained from the gene expression data; an ECM associated signature (e.g., a gene group score generated using two or more, such as 2, 3, 4, 5, 6, or more genes from the ECM associated gene group of Table 1); an Angiogenesis signature (e.g., a gene group score generated using two or more, such as 2, 3, 4, 5, 6, or more genes from the Angiogenesis gene group of Table 1); a Proliferation rate signature (e.g., a gene group score generated using two or more, such as 2, 3, 4, 5, 6, or more genes from the Proliferation rate gene group of Table 1); and a similarity score produced by comparing the RC TME type identified for the subject to gene group scores of RC TME type B and/or RC TME type C gene group scores from other subjects. In some embodiments, the similarity score is produced by by comparing the gene group scores of the RC TME signature of the subject to an average of gene group scores of a plurality of RC TME signatures from RC TME type B samples and/or an average of gene group scores of a plurality of RC TME signatures from RC TME type C samples. In some embodiments subjects are identified as “TO non-responders” are excluded as inputs from the machine learning algorithms. In some embodiments, an ECM associated signature comprises gene enrichment scores for 1, 2, 3, 4, 5, 6, 7, 8, 9, or 10 genes listed in Table 1. In some embodiments, an Angiogenesis signature comprises gene enrichment scores for 1, 2, 3, 4, 5, 6, 7, 8, 9, 10, 11, 12, 13, 14, or 15 genes listed in Table 1. In some embodiments, a Proliferation rate signature comprises gene enrichment scores for 1, 2, 3, 4, 5, 6, 7, 8, 9, 10, 11, 12, 13, 14, or 15 genes listed in Table 1.

A responder score (e.g., an TO responder score) produced for a subject is then compared to a specified threshold in order to determine whether or not a subject is likely to respond to an immuno-therapeutic agent. In some embodiments, the specified threshold is used to determine (e.g., classify) a subject as being “IO-low”, “IO-medium”, or “IO-high”. The value of the specified threshold may range from between 0.2 to about 0.8 units (e.g., 0.2, 0.3, 0.4, 0.5, 0.6, 0.7, 0.8, or any unit value therebetween). In some embodiments, a specified threshold ranges from about 0 to about 1 units. In some embodiments, a specified threshold is 0.2, 0.3, 0.4, 0.5, 0.6, 0.7, 0.8, 0.9, or 1.0. In some embodiments, a responder score is used to identify the subject as “IO-low” when the responder score is ≤0.05; “IO-medium” when the responder score is ≥0.05, or <0.5; or “IO-high” when the responder score is ≥0.5. In some embodiments, a subject identified as having a responder score above the specified threshold is identified as being likely to respond to treatment with an TO agent. In some embodiments, a subject having a responder score that is below a specified threshold (e.g., less than 0.5) is unlikely to respond positively to an IO therapy (e.g., the IO agent is unlikely to be therapeutically effective in the subject). In some embodiments, a responder score equal to or greater than 0.5 indicates that a subject is likely to respond positively to an IO therapy (e.g., the IO agent is unlikely to be therapeutically effective in the subject).

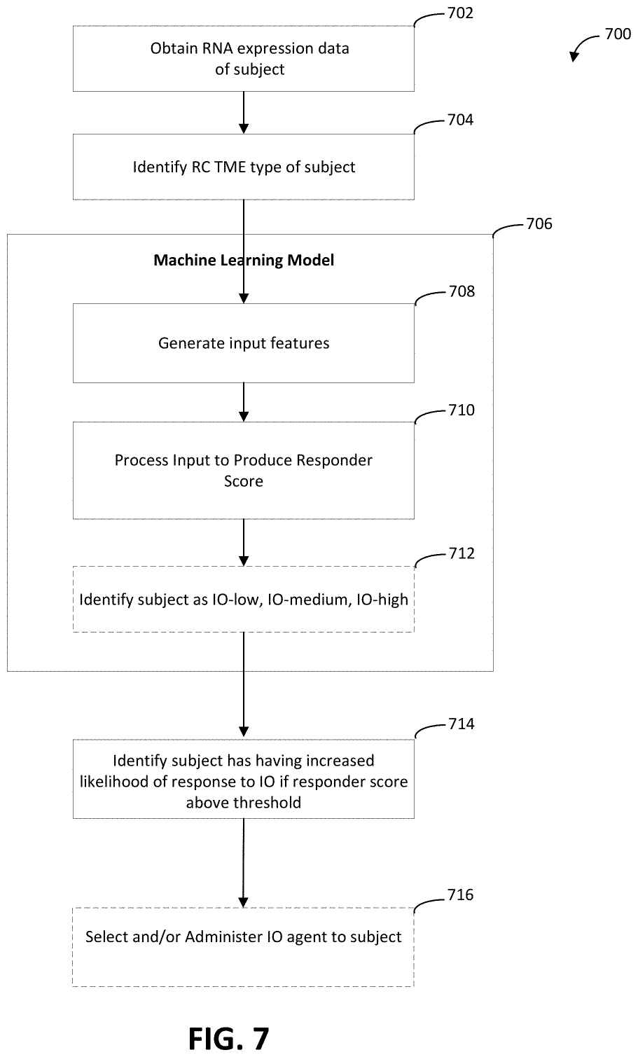

Turning to the Figures, FIG. 7 provides a description of one example of a process for using a computer hardware processor for predicting the likelihood of a subject responding to an immuno-oncology (IO) agent, 700. First, sequencing data of a subject is obtained in act 702. Methods of obtaining sequencing data are described throughout the specification including in the section entitled Sequencing Data and Gene Expression Data. Sequencing data may be processed to obtain gene expression data. The gene expression data is then used to identify the RC TME type of the subject in act 704. In some embodiments, the gene expression data is RNA expression data. In some embodiments, the RC TME type of the subject is identified by using the RNA expression data to generate an RC TME signature of the subject (e.g., using RNA levels of the expression data to generate one or more gene group scores for one or more gene groups listed in Table 1), and then using the RC TME signature of the subject to identify the RC TME type of the subject.

After the RC TME type of the subject has been identified, a machine learning model is used to obtain an output indicating a responder score (the responder score indicative of a likelihood that the subject responds to an IO agent) in process 706. In some embodiments, the obtaining comprises generating, using RNA expression data that has been obtained from the subject, a set of input features, the set of input features comprising at least two (e.g., 2, 3, 4, 5, or 6) of the following features: an RC TME type for the subject; RNA expression levels for one or more of the following genes: PD1, PD-L1, and PD-L2; an ECM associated signature for the subject; an Angiogenesis signature for the subject; a Proliferation rate signature for the subject; and a similarity score indicative of a similarity of an RC TME signature for the subject to RC TME signatures associated with RC TME type B and/or RC TME Type C samples in act 708.

In some embodiments, generating the set of input features comprises determining the RNA expression levels for one or more of the following genes: PD1, PD-L1, and PD-L2. In some embodiments, generating the set of input features comprises determining the ECM associated signature for the subject using the RNA expression data by performing ssGSEA on the RNA expression data for at least three (e.g., 3, 4, 5, 6, or all) of the “ECM associated signature” genes listed in Table 1 to produce an ECM associated gene group score. In some embodiments, generating the set of input features comprises determining the Angiogenesis signature for the subject using the RNA expression data by performing ssGSEA on the RNA expression data for at least three (e.g., 3, 4, 5, 6, or all) of the “Angiogenesis” genes listed in Table 1 to produce an Angiogenesis gene group score. In some embodiments, generating the set of input features comprises determining the Proliferation rate signature for the subject using the RNA expression data by performing ssGSEA on the RNA expression data for at least three (e.g., 3, 4, 5, 6, or all) of the “Proliferation rate” genes listed in Table 1 to produce a Proliferation rate gene group score. In some embodiments, generating the set of input features comprises determining the similarity score by comparing the gene group scores of the RC TME signature of the subject to an average of gene group scores of a plurality of RC TME signatures from RC TME type B samples and/or an average of gene group scores of a plurality of RC TME signatures from RC TME type C samples.

Next, the set of input features generated in act 708 is used as input for a machine learning model comprising a gradient boosting model, which is used to obtain a corresponding output indicating a responder score in act 710. In some embodiments, a gradient boosting model comprises a CatBoost classifier.

The machine learning model used to generate an immuno-oncology (IO) responder score may be of any suitable type. For example, in some embodiments, the machine learning model may be a gradient boosted machine learning model. Non-limiting examples of a gradient boosted machine learning model include an XGBoost model, a LightGBM model, a CatBoost model, an Adaboost model, or a random forest model. However, the machine learning model may be of any other suitable type and, for example, may be a non-linear regression model (e.g., a logistic regression model), a neural network model, a support vector machine, a Gaussian mixture model, a random forest model, a decision tree model, or any other suitable type of machine learning model, as aspects of the technology described herein are not limited in this respect.

In some embodiments, the machine learning model may comprise between 10 and 100 parameters, between 100 and 1000 parameters, between 1000 and 10,000 parameters, between 10,000 and 100,000 parameters or more than 100K parameters. Processing input data with a machine learning model comprises performing calculations using values of the machine learning model parameters and the values of the input to the machine learning model to obtain the corresponding output. Such calculations may involve hundreds, thousands, tens of thousands, hundreds of thousands or more calculations, in some embodiments.

In some embodiments, the machine learning model may include multiple parameters whose values may be estimated using training data. The process of estimating parameter values using training data is termed “training”. In some embodiments, a machine learning model may include one or more hyperparameters in addition to the multiple parameters. Values of the hyperparameters may be estimated during training as well.

After a responder score has been output from the machine learning model, the subject may optionally be identified as “IO-low”, IO-medium”, or “IO-high” based upon the responder score in act 712. The responder score of the subject is then compared to a specified threshold in order to determine whether or not a subject is likely to respond to an immuno-therapeutic agent in act 714. The value of the specified threshold may vary. In some embodiments, the value of the specified threshold ranges from about 0.2 to about 0.8 units. In some embodiments, the specified threshold is 0.5 units. If a subject is identified as having a responder score above the specified threshold, then the subject is identified as having an increase likelihood of responding to an IO agent. In some embodiments, the subject is identified as being “IO-low” when the subject has a responder score that is less than ≤0.05. In some embodiments, the subject is identified as being “IO-medium” when the responder score is ≥0.05 and <0.5. In some embodiments, the subject is identified as being “IO-high” when the responder score is ≥0.5. However, it should be appreciated that depending on the value of the specified threshold, a subject having a responder score of ≤0.5 may be identified as being “IO-high” (e.g., if the threshold value is 0.4, then a subject identified as having a responder score >0.4 will be identified as being “IO-high”). In some embodiments, the method further comprises administering an IO therapy to the subject in act 716.

In some aspects, the disclosure provides a method for predicting the likelihood of a subject responding to a tyrosine kinase inhibitor (TKI). In some embodiments, the method comprises generating, using RNA expression data that has been obtained from a subject, a set of input features, the set of input features comprising at least two (e.g., 2, 3, or 4) of the following features: a Macrophage signature for the subject; an Angiogenesis signature for the subject; a Proliferation rate signature for the subject; and a similarity score indicative of a similarity of an RC TME signature for the subject to RC TME signatures associated with RC TME type B samples. In some embodiments, the set of input features is used to train a machine learning model to obtain a corresponding output indicating a responder score, which is indicative of a likelihood that the subject responds to the TKI. In some embodiments, the machine learning model comprises a logistic regression model.

In some embodiments, a Macrophage signature comprises a Macrophages gene group score generated using RNA levels for 1, 2, 3, 4, 5, 6, 7, or 8 of the Macrophages group genes listed in Table 1. In some embodiments, an Angiogenesis signature comprises an Angiogenesis gene group score for 1, 2, 3, 4, 5, 6, 7, 8, 9, 10, 11, 12, 13, 14, or 15 Angiogenesis genes listed in Table 1. In some embodiments, a Proliferation rate signature comprises gene enrichment scores for 1, 2, 3, 4, 5, 6, 7, 8, 9, 10, 11, 12, 13, 14, or 15 genes listed in Table 1.

A responder score (e.g., a TKI responder score) produced for a subject is then compared to a specified threshold in order to determine whether or not a subject is likely to respond to a TKI. In some embodiments, the specified threshold is used to determine (e.g., classify) a subject as being “TKI-low”, “TKI-medium”, or “TKI-high”. The value of the specified threshold may range from between 0.1 to about 1.5 units (e.g., 0.2, 0.3, 0.4, 0.5, 0.6, 0.7, 0.8, 0.9, 1.0, 1.1, 1.2, 1.3, 1.4, or 1.5, or any unit value therebetween). In some embodiments, a specified threshold ranges from about 0 to about 1 units. In some embodiments, a specified threshold is 0.25, 0.5, 0.75, 0.85, 0.95, or 1.0. In some embodiments, a subject identified as having a responder score above the specified threshold is identified as being likely to respond to treatment with a TKI agent. In some embodiments, a subject having a responder score that is less than 0.6 is unlikely to respond positively to a TKI (e.g., the TKI is unlikely to be therapeutically effective in the subject). In some embodiments, a responder score equal to or greater than 0.6 indicates that a subject is likely to respond positively to a TKI (e.g., the TKI is unlikely to be therapeutically effective in the subject). In some embodiments, a responder score is used to identify the subject as “TKI-low” when the responder score is ≤0.75; “TKI-medium” when the responder score is ≥0.75, or <0.95; or “TKI-high” when the responder score is ≥0.95.

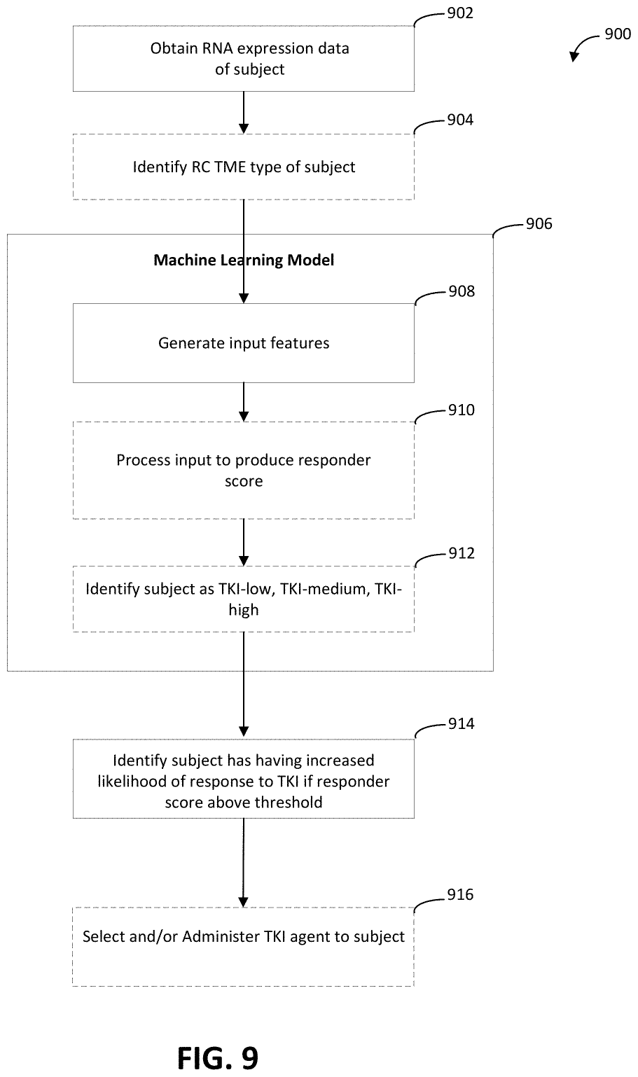

FIG. 9 provides a description of one example of a process for using a computer hardware processor to perform a method 900 for predicting the likelihood of a subject responding to a tyrosine kinase inhibitor (TKI), according to some embodiments of the technology described herein. First, RNA expression data of the subject is obtained, 902. Methods of obtaining sequencing data are described throughout the specification including in the section entitled Sequencing Data and Gene Expression Data. The sequencing data is processed to obtain RNA expression data. Optionally, the RC TME type of the subject is identified using the RNA expression data in act 904. In some embodiments, the gene expression data is RNA expression data. In some embodiments, the RC TME type of the subject is identified by using the RNA expression data to generate an RC TME signature of the subject (e.g., using RNA levels of the expression data to generate one or more gene group scores for one or more gene groups listed in Table 1), and then using the RC TME signature of the subject to identify the RC TME type of the subject.

After the RC TME type of the subject has been identified, a machine learning model is used to obtain an output indicating a responder score (the responder score indicative of a likelihood that the subject responds to a TKI) from a set of input features in process 906. In some embodiments, the obtaining comprises generating, using RNA expression data that has been obtained from the subject, a set of input features, the set of input features comprising at least two (e.g., 2, 3, or 4) of the following features: a Macrophage associated signature for the subject; an Angiogenesis signature for the subject; a Proliferation rate signature for the subject; and a similarity score indicative of a similarity of an RC TME signature for the subject to RC TME signatures associated with RC TME type B samples in, act 908.

In some embodiments, generating the set of input features comprises determining the Macrophage signature for the subject using the RNA expression data by performing ssGSEA on the RNA expression data for at least three (e.g., 3, 4, 5, 6, or all) of the “Macrophages” genes listed in Table 1 to produce a Macrophage gene group score. In some embodiments, generating the set of input features comprises determining the Angiogenesis signature for the subject using the RNA expression data by performing ssGSEA on the RNA expression data for at least three (e.g., 3, 4, 5, 6, or all) of the “Angiogenesis” genes listed in Table 1 to produce an Angiogenesis gene group score. In some embodiments, generating the set of input features comprises determining the Proliferation rate signature for the subject using the RNA expression data by performing ssGSEA on the RNA expression data for at least three (e.g., 3, 4, 5, 6, or all) of the “Proliferation rate” genes listed in Table 1 to produce a Proliferation rate gene group score. In some embodiments, generating the set of input features comprises determining the similarity score by comparing the gene group scores of the RC TME signature of the subject to an average of gene group scores of a plurality of RC TME signatures from RC TME type B samples.

Next, the set of input features generated in act 908 is used as input for a machine learning model comprising a logistic regression model, which is used to obtain a corresponding output indicating a responder score in act 910.

The machine learning model used to generate a TKI responder score may be of any suitable type. For example, in some embodiments, the machine learning model may be a non-linear regression model (e.g., a logistic regression model). However, the machine learning model may be of any other suitable type and, for example, may be a gradient boosting model (e.g., XGBoost, CatBoost, LightGBM, etc.), a neural network model, a support vector machine, a Gaussian mixture model, a random forest model, a decision tree model, or any other suitable type of machine learning model, as aspects of the technology described herein are not limited in this respect.

In some embodiments, the machine learning model may comprise between 10 and 100 parameters, between 100 and 1000 parameters, between 1000 and 10,000 parameters, between 10,000 and 100,000 parameters or more than 100K parameters. Processing input data with a machine learning model comprises performing calculations using values of the machine learning model parameters and the values of the input to the machine learning model to obtain the corresponding output. Such calculations may involve hundreds, thousands, tens of thousands, hundreds of thousands or more calculations, in some embodiments.

In some embodiments, the machine learning model may include multiple parameters whose values may be estimated using training data. The process of estimating parameter values using training data is termed “training”. In some embodiments, a machine learning model may include one or more hyperparameters in addition to the multiple parameters. Values of the hyperparameters may be estimated during training as well.

After a responder score has been output from the machine learning model, the subject may optionally be identified as “TKI-low”, “TKI-medium”, or “TKI-high” based upon the responder score in act 912.

The responder score of the subject is then compared to a specified threshold in order to determine whether or not a subject is likely to respond to an immuno-therapeutic agent in act 914. The value of the specified threshold may vary. In some embodiments, the value of the specified threshold ranges from about 0.1 to about 1.5 units. In some embodiments, the specified threshold is 0.6 units. If a subject is identified as having a responder score above the specified threshold, then the subject is identified as having an increase likelihood of responding to a TKI. In some embodiments, the subject is identified as being “TKI-low” when the subject has a responder score that is less than ≤0.75. In some embodiments, the subject is identified as being “TKI-medium” when the responder score is ≥0.75 and <0.95. In some embodiments, the subject is identified as being “TKI-high” when the responder score is ≥0.95. However, it should be appreciated that depending on the value of the specified threshold, a subject having a responder score of <0.95 may be identified as being “TKI-high” (e.g., if the threshold value is 0.6, then a subject identified as having a responder score >0.6 will be identified as being “TKI-high”). In some embodiments, the method further comprises administering a TKI to the subject in act 916.

### Immuno-Oncology (IO)/TKI Decision Tree

Aspects of the disclosure relate to methods for selecting one or more therapeutic agents for a subject having a renal cancer (e.g., ccRCC). The disclosure is based, in part, on methods that identify the likelihood of a patient's response to either an immune-oncology (IO) agent and/or a tyrosine kinase inhibitor (TKI) using RNA sequencing data obtained from the subject to produce one or more responder scores (e.g., a responder score for IO, a responder score for TKI, etc.) for the subject. Without wishing to be bound by any particular theory, methods of selecting a therapeutic agent described herein provide physicians increased confidence in identifying classes of therapeutic agents, or combinations of therapeutic agents, to which their patients have an increased likelihood of responding (and conversely, allow physicians to avoid prescribing therapeutic agents to which their patients are unlikely to respond), thereby improving patient care technology. A schematic depicting an example of methods described in this section is provided in FIG. 14.

In some aspects, the disclosure provides a method for identifying one or more therapeutic agents for administration to a subject having renal cancer, the method comprising: generating an International Metastatic RCC Database Consortium (IMDC) Risk Score for the subject; when the subject is identified as having a Poor IMDC Risk Score, identifying a combination of immuno-oncology (IO) agent and TKI as the one or more therapeutic agents for administration to the subject; when the subject is identified as having a Favorable or Intermediate IMDC Risk Score, generating: an IO responder score according to a method as described herein; a TKI responder score according to a method as described herein; and identifying the one or more therapeutic agents for the subject using the IO responder score and the TKI responder score.

An IMDC Risk Score may be calculated using any suitable method, for example as described by Guida et al. Oncotarget. 2020; 11:4582-4592. Typically, an IMDC Risk Score classifies patients as one of the following categories: “Good” (also referred to as “Favorable”), “Intermediate”, or “Poor”, based upon six negative clinical prognostic factors: performance status (e.g., a score of <80 for Karnofsky Performance Status [KPS]); a hemoglobin level <low normal level [LNL]), the time from diagnosis to start of systemic treatment [DTT] (<1 year), a corrected serum calcium level (>upper normal level [UNL]), neutrophil count (>UNL), and platelet count (>UNL)). Patients lacking these negative factors are identified as having a “good” (or “favourable”) prognosis; patients presenting 1 or 2 of the factors have an “intermediate” risk of death; and patients with 3 or more factors have an expected “poor” risk outcome. In some embodiments of methods described by the disclosure, when a sample from a subject identified as having a “poor” IMDC Risk Score, a combination of TKI and IO agents are selected for the subject without further analysis of an IO responder score or a TKI responder score.

In some embodiments, the methods comprise a step of generating a TKI responder score for a subject having an “Intermediate” or “Favourable” IMDC Risk Score. Methods of generating IO responder scores are described elsewhere in the specification, for example in FIG. 9 and the section entitled “Responder Scores”. In some embodiments, a subject is identified as having a TKI responder score between 0 and 1 (e.g., any value between and including 0 and 1). In some embodiments, the subject is identified as being “TKI-low” when the subject has a responder score that is less than ≤0.75. In some embodiments, the subject is identified as being “TKI-medium” when the responder score is ≥0.75 and <0.95. In some embodiments, the subject is identified as being “TKI-high” when the responder score is ≥0.95.

In some embodiments, the methods comprise a step of generating an IO responder score for a subject having an “Intermediate” or “Favourable” IMDC Risk Score. In some embodiments, prior to generating the IO responder score, it is determined whether the subject is an “IO non-responder”. Methods of identifying “IO non-responders” are described elsewhere in the disclosure, for example in the section entitled “Responder Scores”. In some embodiments, identifying the subject as an “IO non-responder” comprises identifying that the subject (e.g., a biological sample obtained from the subject) has one or more of the following biomarkers: high Ploidy (e.g., as calculated by RTumor bioinformatics analysis), a high Myogenic Signature (e.g., as described throughout the specification for example in the section entitled Myogenesis Signature), RC TME type E (as determined by methods described throughout the specification), presence of mTOR activating mutations, or presence of mutations in antigen presentation machinery. In some embodiments, the method comprises selecting one or more TKIs for a subject identified as an “IO non-responder”.

If the subject is not identified as an “IO non-responder”, an IO responder score is generated for the subject. Methods of generating IO responder scores are described elsewhere in the specification, for example in FIG. 7 and the section entitled “Responder Scores”. In some embodiments, a subject is identified as having an IO responder score between 0 and 1 (e.g., any value between and including 0 and 1). In some embodiments, the subject is identified as being “IO-low” when the subject has a responder score that is less than ≤0.05. In some embodiments, the subject is identified as being “IO-medium” when the responder score is ≥0.05 and <0.5. In some embodiments, the subject is identified as being “IO-high” when the responder score is ≥0.5.

In some embodiments, the method comprises selecting (or providing a recommendation to select) one or more therapeutic agents for the subject (e.g., producing a report recommending selection of one or more therapeutic agents for the subject) using the TKI and IO responder scores. In some embodiments, when a subject is identified as “TKI-low” and “IO-low”, a TKI agent is selected for the subject. In some embodiments, when a subject is identified as “TKI-low” and “IO-low”, a combination of a TKI agent and an IO agent is selected for the subject. In some embodiments, when a subject is identified as “TKI-medium” and “IO-low” or “IO-medium”, a combination of a TKI agent and an IO agent is selected for the subject. In some embodiments, when a subject is identified as “TKI-medium” and “IO-low” or “IO-medium”, a TKI agent is selected for the subject. In some embodiments, when a subject is identified as “TKI-medium” and “IO-high” a combination of a TKI agent and an IO agent is selected for the subject. In some embodiments, when a subject is identified as “TKI-high” and “IO-low” or “IO-medium”, a TKI agent is selected for the subject. In some embodiments, when a subject is identified as “TKI-high” and “IO-high” a combination of a TKI agent and an IO agent is selected for the subject. In some aspects, the methods further comprise a step of administering the identified one or more therapeutic agents (e.g., TKI agent, or combination of TKI agent and IO agent) to the subject. Methods of administering a TKI agent or a combination of TKI agent and IO agent to a subject are described further herein, for example in the section entitled “Therapeutic Indications”.

### Therapeutic Indications

Aspects of the disclosure relate to methods of identifying or selecting a therapeutic agent for a subject based upon determination of the subject's RC TME type and/or responder score (e.g., IO responder score or TKI responder score). The disclosure is based, in part, on the recognition that subjects having RC TME type E have a decreased likelihood of responding to certain therapies (e.g., an IO agent) relative to subjects having other RC TME types but may still respond to other therapies, for example TKIs. The disclosure is based, in part, on the recognition that subjects having RC TME type A or RC TME type B have an increased likelihood of responding to certain therapies (e.g., an IO agent) relative to subjects having other RC TME types.

In some embodiments, the therapeutic agents are immuno-oncology (IO) agents. An IO agent may be a small molecule, peptide, protein (e.g., antibody, such as monoclonal antibody), interfering nucleic acid, or a combination of any of the foregoing. In some embodiments, the IO agents comprise a PD1 inhibitor, PD-L1 inhibitor, or PD-L2 inhibitor. Examples of IO agents include but are not limited to cemiplimab, nivolumab, pembrolizumab, avelumab, durvalumab, atezolizumab, BMS1166, BMS202, etc.

In some embodiments, the therapeutic agents are tyrosine kinase inhibitors (TKIs). A TKI may be a small molecule, peptide, protein (e.g., antibody, such as monoclonal antibody), interfering nucleic acid, or a combination of any of the foregoing. Examples of TKIs include but are not limited to Axitinib (Inlyta®), Cabozantinib (Cabometyx®), Imatinib mesylate (Gleevec®), Dasatinib (Sprycel®), Nilotinib (Tasigna®), Bosutinib (Bosulif®), Sunitinib (Sutent®), etc.

In some embodiments, methods described by the disclosure further comprise a step of administering one or more therapeutic agents to the subject based upon the determination of the subject's RC TME type and/or responder score. In some embodiments, a subject is administered one or more (e.g., 1, 2, 3, 4, 5, or more) IO agents. In some embodiments, a subject is administered one or more (e.g., 1, 2, 3, 4, 5, or more) TKIs. In some embodiments, a subject is administered a combination of one or more IO agents and one or more TKIs.

Aspects of the disclosure relate to methods of treating a subject having (or suspected or at risk of having) RC based upon a determination of the RC TME type of the subject. In some embodiments, the methods comprise administering one or more (e.g., 1, 2, 3, 4, 5, or more) therapeutic agents to the subject. In some embodiments, the therapeutic agent (or agents) administered to the subject are selected from small molecules, peptides, nucleic acids, radioisotopes, cells (e.g., CAR T-cells, etc.), and combinations thereof. Examples of therapeutic agents include chemotherapies (e.g., cytotoxic agents, etc.), immunotherapies (e.g., immune checkpoint inhibitors, such as PD-1 inhibitors, PD-L1 inhibitors, etc.), antibodies (e.g., anti-HER2 antibodies), cellular therapies (e.g. CAR T-cell therapies), gene silencing therapies (e.g., interfering RNAs, CRISPR, etc.), antibody-drug conjugates (ADCs), and combinations thereof.

In some embodiments, a subject is administered an effective amount of a therapeutic agent. “An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons, or for virtually any other reasons.

Empirical considerations, such as the half-life of a therapeutic compound, generally contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally (but not necessarily) based on treatment, and/or suppression, and/or amelioration, and/or delay of a cancer. Alternatively, sustained continuous release formulations of an anti-cancer therapeutic agent may be appropriate. Various formulations and devices for achieving sustained release are known in the art.

In some embodiments, dosages for an anti-cancer therapeutic agent as described herein may be determined empirically in individuals who have been administered one or more doses of the anti-cancer therapeutic agent. Individuals may be administered incremental dosages of the anti-cancer therapeutic agent. To assess efficacy of an administered anti-cancer therapeutic agent, one or more aspects of a cancer (e.g., tumor microenvironment, tumor formation, tumor growth, or RC TME types, etc.) may be analyzed.

Generally, for administration of any of the anti-cancer antibodies described herein, an initial candidate dosage may be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression or amelioration of symptoms occurs or until sufficient therapeutic levels are achieved to alleviate a cancer, or one or more symptoms thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner (e.g., a medical doctor) wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy may be monitored by conventional techniques and assays and/or by monitoring RC TME types as described herein. The dosing regimen (including the therapeutic used) may vary over time.

When the anti-cancer therapeutic agent is not an antibody, it may be administered at the rate of about 0.1 to 300 mg/kg of the weight of the patient divided into one to three doses, or as disclosed herein. In some embodiments, for an adult patient of normal weight, doses ranging from about 0.3 to 5.00 mg/kg may be administered. The particular dosage regimen, e.g., dose, timing, and/or repetition, will depend on the particular subject and that individual's medical history, as well as the properties of the individual agents (such as the half-life of the agent, and other considerations well known in the art).

For the purpose of the present disclosure, the appropriate dosage of an anti-cancer therapeutic agent will depend on the specific anti-cancer therapeutic agent(s) (or compositions thereof) employed, the type and severity of cancer, whether the anti-cancer therapeutic agent is administered for preventive or therapeutic purposes, previous therapy, the patient's clinical history and response to the anti-cancer therapeutic agent, and the discretion of the attending physician. Typically, the clinician will administer an anti-cancer therapeutic agent, such as an antibody, until a dosage is reached that achieves the desired result.

Administration of an anti-cancer therapeutic agent can be continuous or intermittent, depending, for example, upon the recipient's physiological condition, whether the purpose of the administration is therapeutic or prophylactic, and other factors known to skilled practitioners. The administration of an anti-cancer therapeutic agent (e.g., an anti-cancer antibody) may be essentially continuous over a preselected period of time or may be in a series of spaced dose, e.g., either before, during, or after developing cancer.

As used herein, the term “treating” refers to the application or administration of a composition including one or more active agents to a subject, who has a cancer, a symptom of a cancer, or a predisposition toward a cancer, with the purpose to cure, heal, alleviate, relieve, alter, remedy, ameliorate, improve, or affect the cancer or one or more symptoms of RC, or the predisposition toward RC.

Alleviating RC includes delaying the development or progression of the disease, or reducing disease severity. Alleviating the disease does not necessarily require curative results. As used therein, “delaying” the development of a disease (e.g., a cancer) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detected and assessed using clinical techniques known in the art. Alternatively, or in addition to the clinical techniques known in the art, development of the disease may be detectable and assessed based on other criteria. However, development also refers to progression that may be undetectable. For purpose of this disclosure, development or progression refers to the biological course of the symptoms. “Development” includes occurrence, recurrence, and onset. As used herein “onset” or “occurrence” of a cancer includes initial onset and/or recurrence.

Examples of the antibody anti-cancer agents include, but are not limited to, alemtuzumab (Campath), trastuzumab (Herceptin), Ibritumomab tiuxetan (Zevalin), Brentuximab vedotin (Adcetris), Ado-trastuzumab emtansine (Kadcyla), blinatumomab (Blincyto), Bevacizumab (Avastin), Cetuximab (Erbitux), ipilimumab (Yervoy), nivolumab (Opdivo), pembrolizumab (Keytruda), atezolizumab (Tecentriq), avelumab (Bavencio), durvalumab (Imfinzi), and panitumumab (Vectibix).

Examples of an immunotherapy include, but are not limited to, a PD-1 inhibitor or a PD-L1 inhibitor, a CTLA-4 inhibitor, adoptive cell transfer, therapeutic cancer vaccines, oncolytic virus therapy, T-cell therapy, and immune checkpoint inhibitors.

Examples of radiation therapy include, but are not limited to, ionizing radiation, gamma-radiation, neutron beam radiotherapy, electron beam radiotherapy, proton therapy, brachytherapy, systemic radioactive isotopes, and radiosensitizers.

Examples of a surgical therapy include, but are not limited to, a curative surgery (e.g., tumor removal surgery), a preventive surgery, a laparoscopic surgery, and a laser surgery.

Examples of the chemotherapeutic agents include, but are not limited to, R-CHOP, Carboplatin or Cisplatin, Docetaxel, Gemcitabine, Nab-Paclitaxel, Paclitaxel, Pemetrexed, and Vinorelbine. Additional examples of chemotherapy include, but are not limited to, Platinating agents, such as Carboplatin, Oxaliplatin, Cisplatin, Nedaplatin, Satraplatin, Lobaplatin, Triplatin, Tetranitrate, Picoplatin, Prolindac, Aroplatin and other derivatives; Topoisomerase I inhibitors, such as Camptothecin, Topotecan, irinotecan/SN38, rubitecan, Belotecan, and other derivatives; Topoisomerase II inhibitors, such as Etoposide (VP-16), Daunorubicin, a doxorubicin agent (e.g., doxorubicin, doxorubicin hydrochloride, doxorubicin analogs, or doxorubicin and salts or analogs thereof in liposomes), Mitoxantrone, Aclarubicin, Epirubicin, Idarubicin, Amrubicin, Amsacrine, Pirarubicin, Valrubicin, Zorubicin, Teniposide and other derivatives; Antimetabolites, such as Folic family (Methotrexate, Pemetrexed, Raltitrexed, Aminopterin, and relatives or derivatives thereof); Purine antagonists (Thioguanine, Fludarabine, Cladribine, 6-Mercaptopurine, Pentostatin, clofarabine, and relatives or derivatives thereof) and Pyrimidine antagonists (Cytarabine, Floxuridine, Azacitidine, Tegafur, Carmofur, Capacitabine, Gemcitabine, hydroxyurea, 5-Fluorouracil (5FU), and relatives or derivatives thereof); Alkylating agents, such as Nitrogen mustards (e.g., Cyclophosphamide, Melphalan, Chlorambucil, mechlorethamine, Ifosfamide, mechlorethamine, Trofosfamide, Prednimustine, Bendamustine, Uramustine, Estramustine, and relatives or derivatives thereof); nitrosoureas (e.g., Carmustine, Lomustine, Semustine, Fotemustine, Nimustine, Ranimustine, Streptozocin, and relatives or derivatives thereof); Triazenes (e.g., Dacarbazine, Altretamine, Temozolomide, and relatives or derivatives thereof); Alkyl sulphonates (e.g., Busulfan, Mannosulfan, Treosulfan, and relatives or derivatives thereof); Procarbazine; Mitobronitol, and Aziridines (e.g., Carboquone, Triaziquone, ThioTEPA, triethylenemalamine, and relatives or derivatives thereof); Antibiotics, such as Hydroxyurea, Anthracyclines (e.g., doxorubicin agent, daunorubicin, epirubicin and relatives or derivatives thereof); Anthracenediones (e.g., Mitoxantrone and relatives or derivatives thereof); Streptomyces family antibiotics (e.g., Bleomycin, Mitomycin C, Actinomycin, and Plicamycin); and ultraviolet light.

In some aspects, the disclosure provides a method for treating renal cancer (RC), the method comprising administering one or more therapeutic agents (e.g., one or more anti-cancer agents, such as one or more chemotherapeutic agents) to a subject identified as having a particular RC TME type, wherein the RC TME type of the subject has been identified by method as described by the disclosure.

In some embodiments, a subject has been identified as having RC TME type A, RC TME type B, RC TME type C, RC TME type D, or RC TME type E. In some embodiments, a subject has been identified as having RC TME type B or RC TME type A.

The disclosure is based, in part, on the inventors' recognition that subjects having certain RC TME types are likely to respond well to certain immunotherapies (e.g., immune checkpoint inhibitors, such as pembrolizumab or nivolumab, or TKIs). Dosing of immuno-oncology agents is well-known, for example as described by Louedec et al. Vaccines (Basel). 2020 December; 8(4): 632. For example, dosages of pembrolizumab, for example, include administration of 200 mg every 3 weeks or 400 mg every 6 weeks, by infusion over 30 minutes. Dosing of TKIs is also well-known, for example as described by Gerritse et al. Cancer Treat Rev. 2021 June; 97:102171. doi: 10.1016/j.ctrv.2021.102171. Combination dosing of TKIs and IO agents is also known, for example as described by Rassy et al. Ther Adv Med Oncol. 2020; 12: 1758835920907504.

Aspects of the disclosure are based on the inventors' recognition that subjects having certain RC TME types are unlikely to respond well to certain therapeutic agents, such as immunotherapeutic agents or TKIs. Thus, in some embodiments, the therapeutic agent comprises a therapeutic agent other than an immunotherapy when the subject has been identified as having an RC TME type E, or when the subject has been identified as a “clear IO non-responder”. In some embodiments, the other therapeutic agent is a TKI.

### Reports

In some aspects, methods disclosed herein comprise generating a report for assisting with the preparation of recommendation for prognosis and/or treatment. The generated report can provide summary of information, so that the clinician can identify the RC TME type or suitable therapy. The report as described herein may be a paper report, an electronic record, or a report in any format that is deemed suitable in the art. The report may be shown and/or stored on a computing device known in the art (e.g., handheld device, desktop computer, smart device, website, etc.). The report may be shown and/or stored on any device that is suitable as understood by a skilled person in the art.

In some embodiments, methods disclosed herein can be used for commercial diagnostic purposes. For example, the generated report may include, but is limited to, information concerning expression levels of one or more genes from any of the gene groups described herein, clinical and pathologic factors, patient's prognostic analysis, predicted response to the treatment, classification of the RC TME environment (e.g., as belonging to one of the types described herein), the alternative treatment recommendation, and/or other information. In some embodiments, the methods and reports may include database management for the keeping of the generated reports. For instance, the methods as disclosed herein can create a record in a database for the subject (e.g., subject 1, subject 2, etc.) and populate the specific record with data for the subject. In some embodiments, the generated report can be provided to the subject and/or to the clinicians. In some embodiments, a network connection can be established to a server computer that includes the data and report for receiving or outputting. In some embodiments, the receiving and outputting of the date or report can be requested from the server computer.

### Computer Implementation

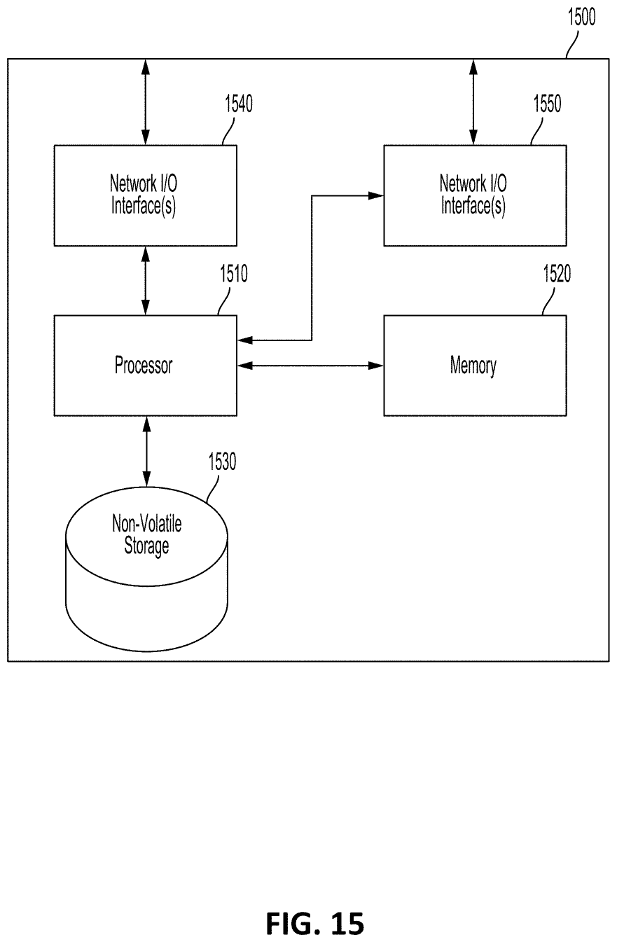

An illustrative implementation of a computer system 1500 that may be used in connection with any of the embodiments of the technology described herein (e.g., such as the method of FIG. 1) is shown in FIG. 15. The computer system 1500 includes one or more processors 1510 and one or more articles of manufacture that comprise non-transitory computer-readable storage media (e.g., memory 1520 and one or more non-volatile storage media 1530). The processor 1510 may control writing data to and reading data from the memory 1520 and the non-volatile storage device 1530 in any suitable manner, as the aspects of the technology described herein are not limited to any particular techniques for writing or reading data. To perform any of the functionality described herein, the processor 1510 may execute one or more processor-executable instructions stored in one or more non-transitory computer-readable storage media (e.g., the memory 1520), which may serve as non-transitory computer-readable storage media storing processor-executable instructions for execution by the processor 1510.

Computing device 1500 may also include a network input/output (I/O) interface 1540 via which the computing device may communicate with other computing devices (e.g., over a network), and may also include one or more user I/O interfaces 1550, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel. It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

### EXAMPLES

### Example 1

Kidney cancer is among the top 10 most frequently diagnosed cancers worldwide, with clear cell renal cell carcinoma (ccRCC) comprising ˜75% of all cases. While the emergence of combination strategies of immuno-oncology (IO) agents with anti-angiogenic tyrosine kinase inhibitors (TKI) has significantly improved the clinical outcomes in this patient population, currently no reliable biomarkers exist to guide treatment decisions.

This example describes a novel approach that integrated whole exome and transcriptome sequencing data from ccRCC samples (n=1,527) to classify the RCC tumor microenvironment (TME) into five major types (referred to as RCC types or RCTs) with distinct immunological composition: “immune-enriched, fibrotic (IE/F)”, “immune-enriched (IE)”, “fibrotic (F)”, “desert with metabolic content (D)”, and “desert with high endothelial cell content (D/E)”. In addition, multiple genomic and transcriptomic-based biomarkers that correlated with lack of IO response were identified. Furthermore, machine learning-based algorithms were employed to generate multifaceted IO and TKI responder scores that combined factors including the RCCTs, angiogenesis, proliferation, macrophage signature and the expression of PD-1, PD-L1 and PD-L2 genes.

Data indicate association of the RCT “IE/F” with superior clinical response (41% response rate) in the IO+TKI cohort. Conversely, the RCT “DIE”, characterized by elevated angiogenesis and the absence of immune cell infiltration, responded significantly better to single agent TKI (50% response rate). Among the genomic and transcriptomic biomarkers, activating mutations of genes within the mTOR signaling pathway, mutations in antigen presentation machinery, high ploidy (>4), and a high myogenesis signature were meaningfully enriched in the IO-resistant patients.

Multi-Omics Responder Scores were then retrospectively constructed and validated in multiple public cohorts, including patients treated with sunitinib (Beuselinck, n=53), pazopanib or sunitinib (COMPARZ trial, n=341), avelumab plus axitinib or sunitinib (JAVELIN Renal 101, n=726), atezolizumab or atezolizumab plus bevacizumab (IMmotion 150, n=160), nivolumab (CheckMate 009/010/025, n=172) and a mixed cohort of patients from Washington University (WUSMRCC, n=75). In all cohorts, implementation of the scoring system described herein led to improved progression free survival and overall survival and appeared superior to all currently available approaches.

In conclusion, a machine learning-based multifaceted approach based on genomic and transcriptomic analyses plus TME composition of ccRCC tumors was used to predict response to IO and TKIs. Retrospective analyses of multiple different cohorts supported the clinical utility of these novel biomarkers.

### Example 2

This example describes a novel approach that integrates whole exome and transcriptome sequencing data from ccRCC samples (n˜1,500) to classify the tumor microenvironment (TME) into five major RC types (also referred to as RC TME types) with distinct immunological composition: immune-enriched, fibrotic (IE/F or “type A”), immune-enriched (IE or “type B”), fibrotic (F or “type C”), desert with metabolic content (type “D”), and desert with high endothelial cell content (“type E”). RC TME types were identified using the gene signatures shown in Table 1, which reflected immune and stromal part of tumor and metabolism pathways activity. ccRCC is considered a cancer caused by metabolic changes due to a high frequency of mutations in genes that control aspects of metabolism, such as a VHL mutation in the hypoxia pathway and mutations in the PI3K-AKT-mTOR pathway (MTOR, TSC1/2, PTEN, and MET) that dysregulate the control of growth in response to nutrient levels. Metabolic shift in glycolysis, oxidative phosphorylation, TCA cycle, fatty acid metabolism and other processes have been observed in ccRCC cells and subjects.

For input of the analysis, gene expression data was obtained using standard bioinformatics analysis packages. For example, RNAseq gene expression data was provided in transcripts per million (TPM). In some cases, FPKM/RPKM values were utilized. For all cohorts, only ccRCC samples were selected (other histological types and normal samples were excluded). There were nine datasets (n˜1500) collected from various platforms. In the analysis, 33 gene signatures were used to identify the five RC TME types. The activity of each signature in each sample was measured using a ssGSEA algorithm.

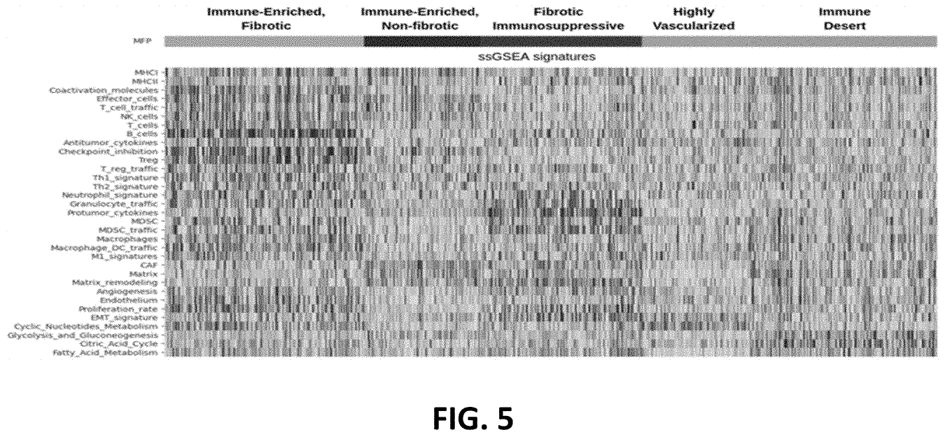

The ssGSEA scores for each signature were medium-scaled inside each cohort. After that, a graph-based clustering algorithm was performed to produce a graph with samples at nodes and correlation of the ssGSEA scores at edges. Each node had 120 neighbors. Then, the Leiden algorithm was applied to the resulting graph and the five RC TME types were identified. A representative heatmap showing clear cell renal carcinoma cancer samples classified into five distinct RC TME types (A, B, C, D, E) based on unsupervised dense clustering of 33 gene expression signatures is shown in FIG. 5.

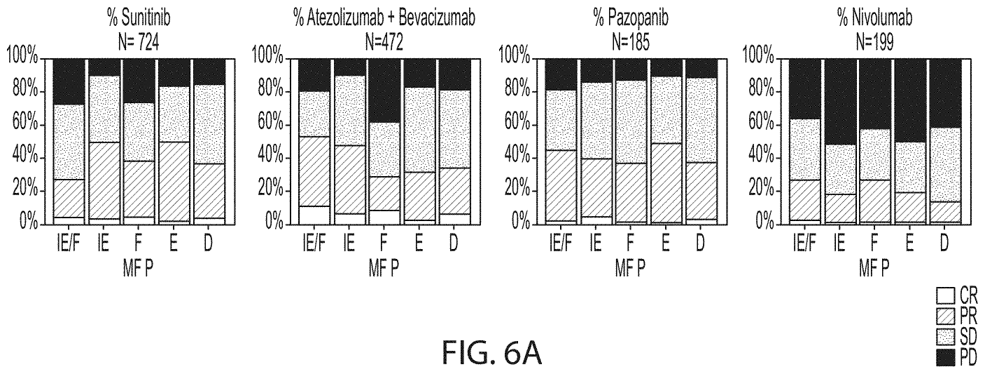

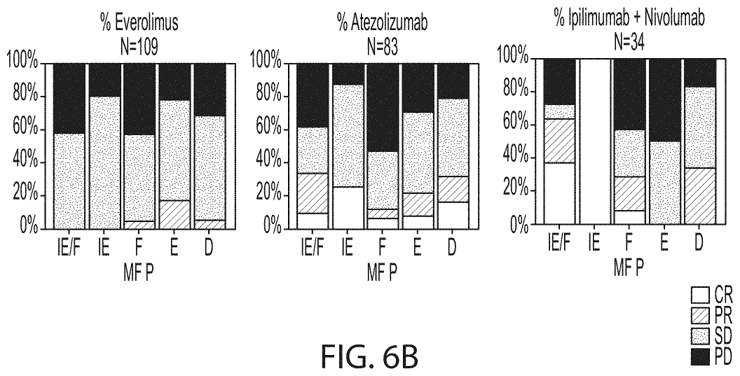

Data, shown in FIGS. 6A-6B, indicate association of the RC TME Type A (also referred to as “IE/F”)) and RC TME type B (also referred to as “IE”) with superior clinical response (>50% response rate) in the IO+TKI (Atezolizumab+Bevacizumab) cohort. Conversely, the RC TME type E, characterized by elevated angiogenesis and the absence of immune cell infiltration, responded significantly better to single agent TKI (˜80% response rate in Sunitinib).

### Example 3

To prescribe the correct treatment for a tumor, a patient typically undergoes a biopsy or a part of the tumor is taken after surgical removal. Then it is sequenced (Targeted Exome-normal, Targeted Exome-tumor, WES-normal, WES-tumor, RNAseq-tumor) and all the necessary molecular functional features are annotated. According to these features, obtained models predict the probability of a response to therapeutic agents. Then, a physician, depending on the patient's previous treatment, current condition, and other clinical factors, decides which drug to use, based upon on the prediction of the models (if there are several alternative therapeutic options).

There are more than a dozen different approved drugs for the treatment of metastatic and advanced clear cell renal carcinoma (ccRCC). When choosing a treatment, a doctor is usually guided by the patient's condition and mono-biomarkers, such as PDL1 IHC, TMB, MSI, which cannot fully account for the complex composition of the tumor as a whole. This example describes a model which is able to assess the likelihood of response to the two most common treatment options for ccRCC: immuno-oncology (IO) agents and tyrosine kinase inhibitors (TKI).

Machine learning-based algorithms that generate multifaceted IO and TKI responder scores were produced. The scores combine factors including the RC TME type, angiogenesis signature, proliferation signature, macrophage signature and the expression of PD-1, PD-L1 and PD-L2 genes. Among the genomic and transcriptomic biomarkers that were meaningfully enriched in the IO-resistant patients were 1) activating mutations of genes within the mTOR signaling pathway, 2) mutations in antigen presentation machinery, 3) high ploidy (>4) and 4) a high Myogenesis signature (described throughout the specification including an Example 4).

To make the models, all currently available public datasets (genomic+transcriptomic) obtained on different platforms of patients with the same diagnosis were collected. The patients in these datasets were treated with the same drug and have known responses or survival rates. All previously published biomarkers of treatment response and other important traits were also collected and used as features for models. Ten public datasets of ccRCC diagnosis and responses to IO and TKI treatments and transcriptomic and genomic data were collected together. For each type of treatment, out of all known features that can be obtained from tumor DNA and RNA using NGS and microarrays, the most important biomarker features were selected using machine learning methods. These features provide the best way to determine the likelihood of a drug response. Thus, for IO, the most important features were determined to be the expression of genes PD1, PDL1, PDL2, and the signatures characterizing ECM associated genes, Angiogenesis and Proliferation rate. For TKI, the most important parameters were determined to be the signatures of Angiogenesis, Macrophages and Proliferation rate.

All features are unified so that they are comparable between datasets. Among all the features in the final model, only those are selected that have the maximum predictive ability and do not introduce noise. Some of the datasets are used for training, and some for validation. The output is a model that provides a single “responder score” predicting the likelihood of a response to treatment (e.g., IO or TKI).

In all cohorts, implementation of scores from the models led to improved progression free survival (PFS) and overall survival (OS) and appeared superior to all currently available approaches. Having NGS cancer biopsy tests and applying these models on the calculated set biomarkers, the physician can assess the likelihood of response to a particular treatment and make a more informed decision.

### IO Model

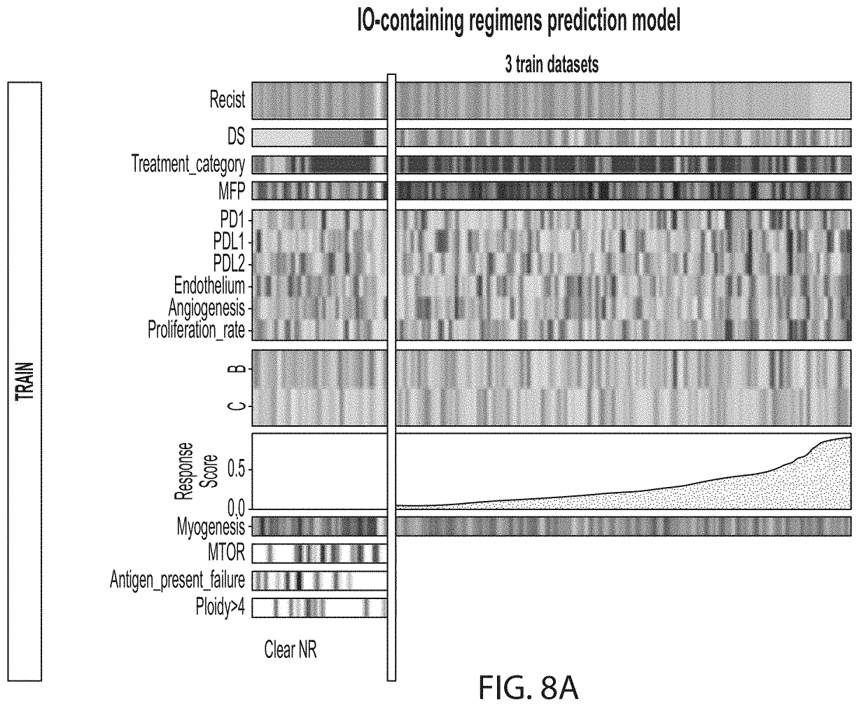

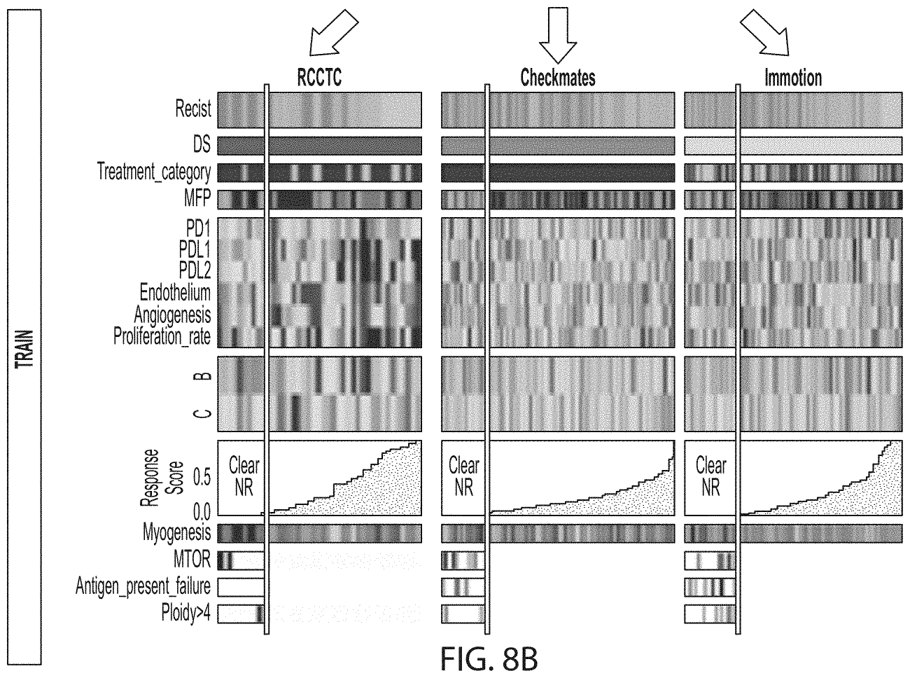

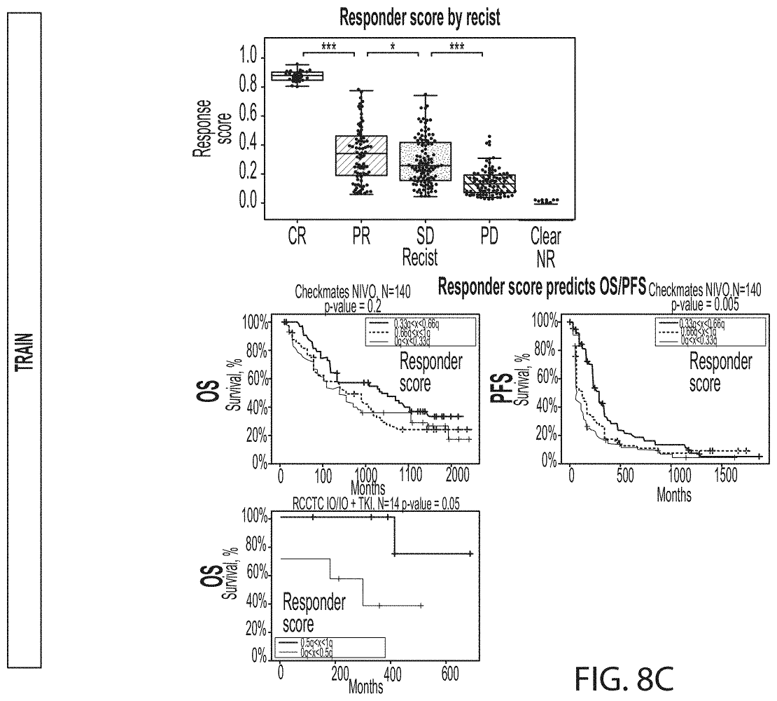

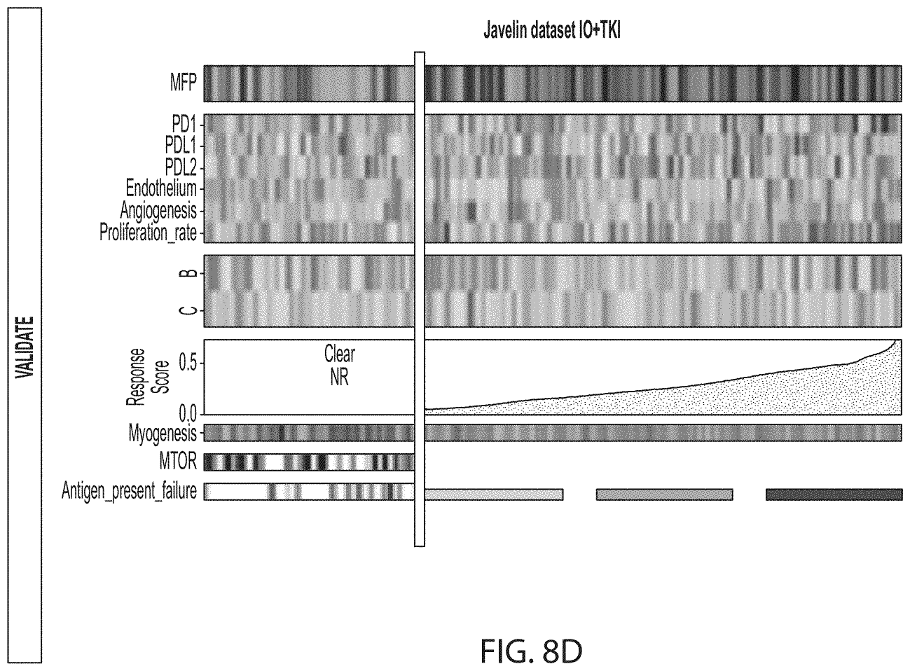

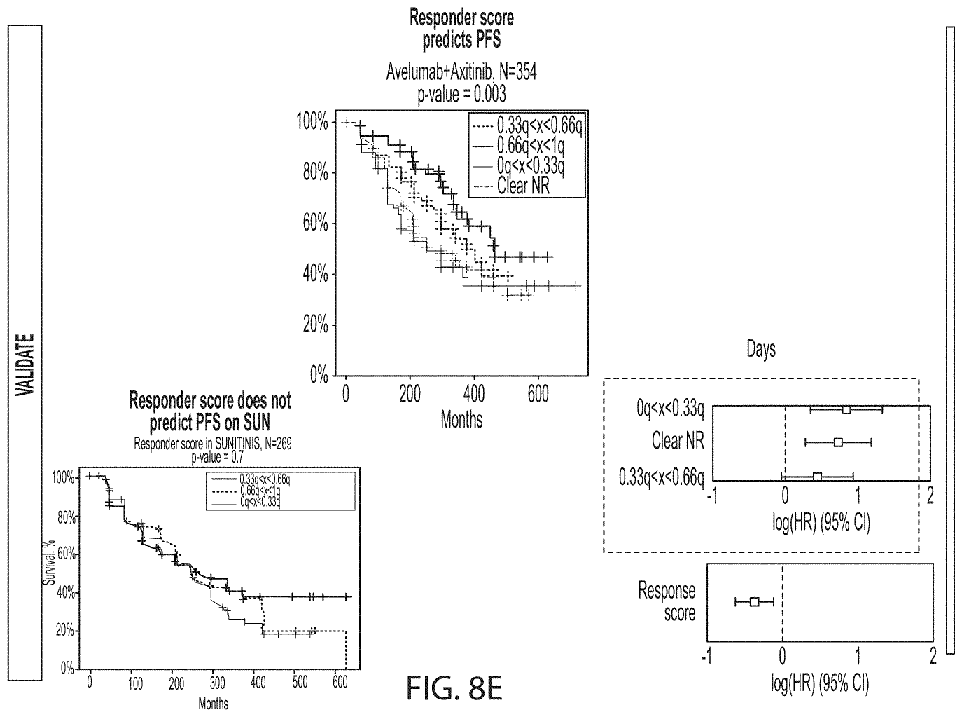

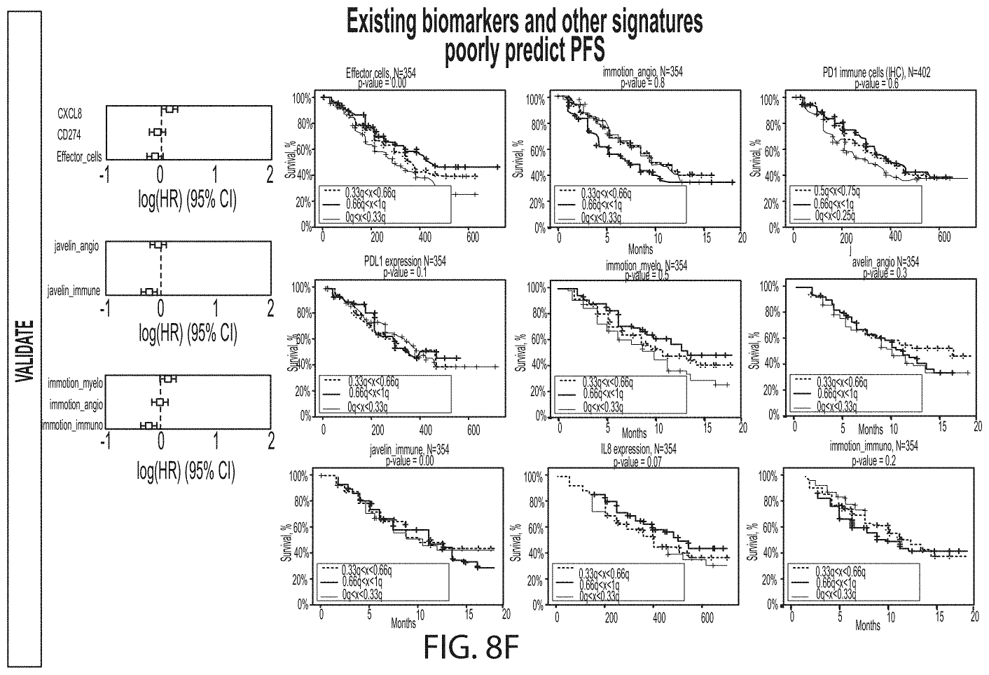

Examples of training and validation of machine learning models for assessing likelihood of response to IO are shown in FIG. 7 and FIGS. 8A-8F. Before model training, a group of biomarkers that were strongly associated with non-response to IO regimens were identified. Patients with these biomarkers were referred to as ‘IO non-responders’. A sample was classified as “TO non-responder” if it met at least one of the following criteria:
Ploidy >4
high Myogenesis signature
RC TME type E
presence of mTOR activating mutations
presence of mutations in antigen presentation machinery

If a sample is classified as ‘IO non-responder’, it was automatically assigned a responder score of 0 and the sample was not used for model training and validation.

A CatBoost classifier model with parameter auto_class_weights set to ‘Balanced’ was used. The model was trained on eight transcriptomic features: expression of PD1, PDL1, PDL2; Endothelium signature, Angiogenesis signature, and Proliferation rate signature. Similarity of a sample to RC TME type “B” and RC TME type “C” was also used for training. Similarity of a sample to a particular RC TME type was calculated as the Spearman correlation coefficient between sample's ssGSEA scores of 33 gene signatures (e.g., based on the gene groups shown in Table 1) and a particular RC TME type's averaged ssGSEA scores of 33 gene signatures (based on the gene groups shown in Table 1). The resulting model score (e.g., responder score) varied from 0 to 1.

The IO model was trained on samples treated with IO-containing regimes from 3 cohorts: WUSMRCC (31 patients IPI+NIVO and 10 patients CABO+NIVO/AXI+PEM), Immotion150 (77 patients ATEZO, 83 ATEZO+BEV), CheckMate25 (172 patients NIVO). For model training patients were selected from these three cohorts with CR (25 patients) and PD (93 patients) RECIST (Response evaluation criteria in solid tumors). These patients were used as two groups for a model to predict. The model was validated on the JAVELIN cohort (354 patients AVE+AXI).

### TKI Model

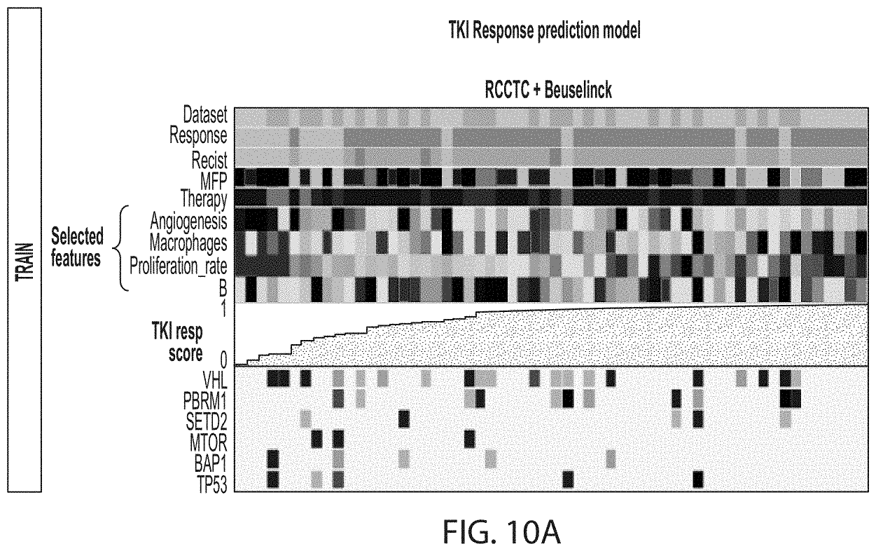

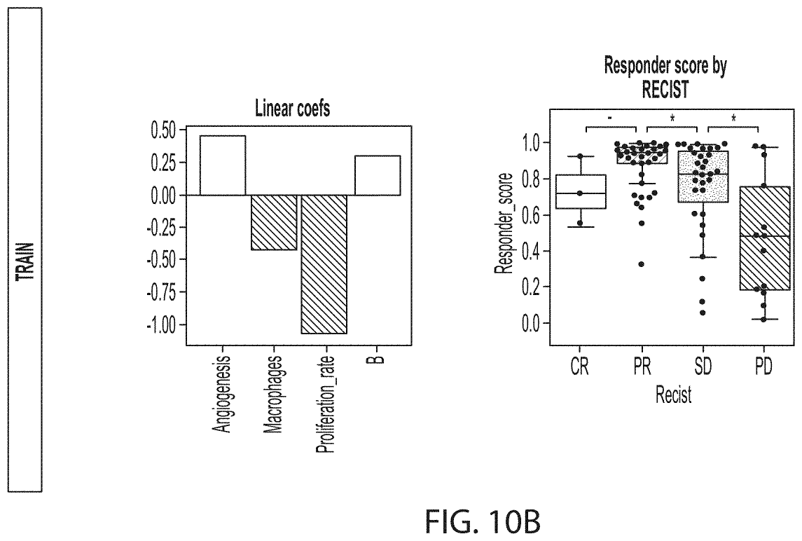

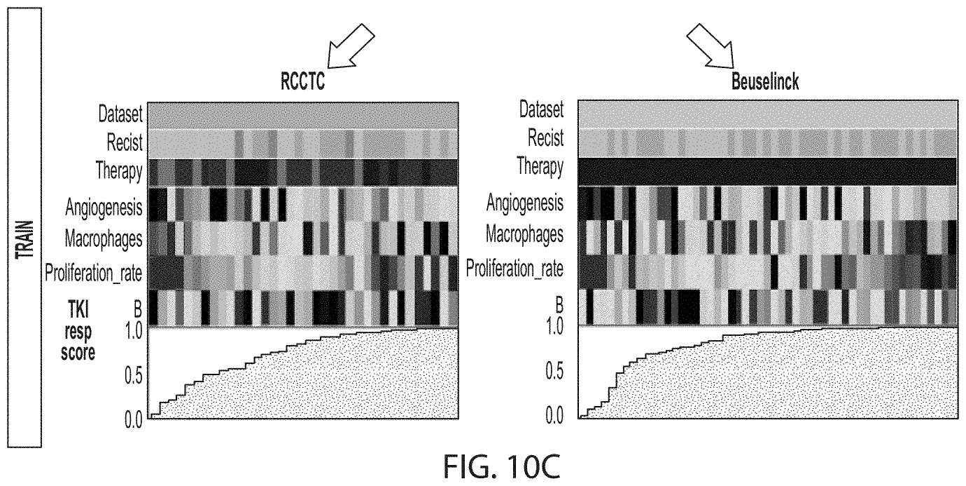

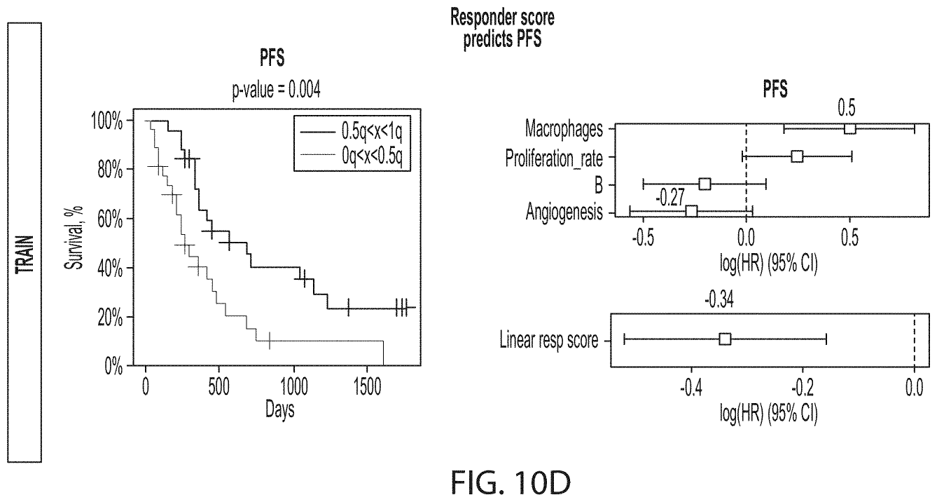

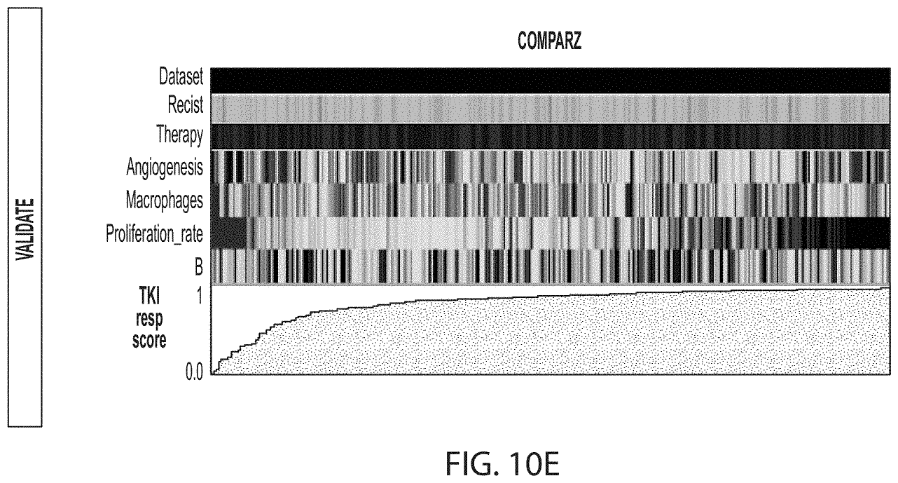

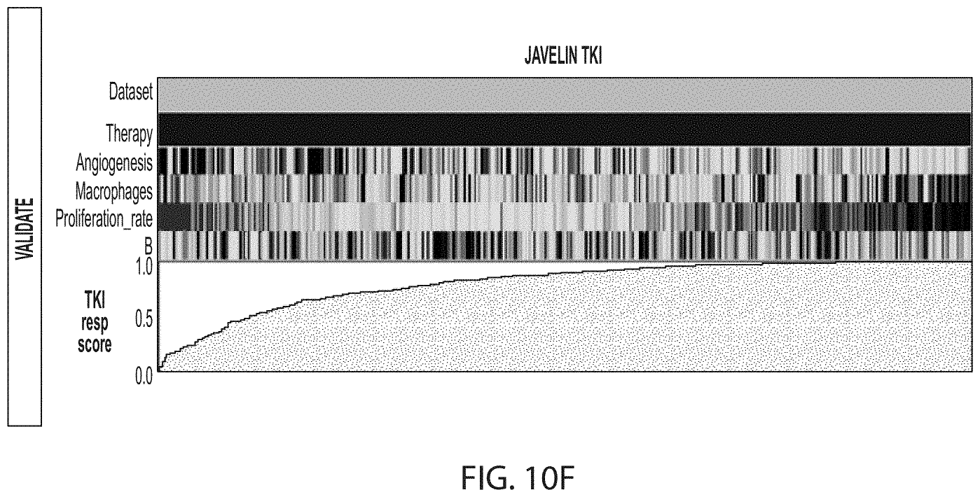

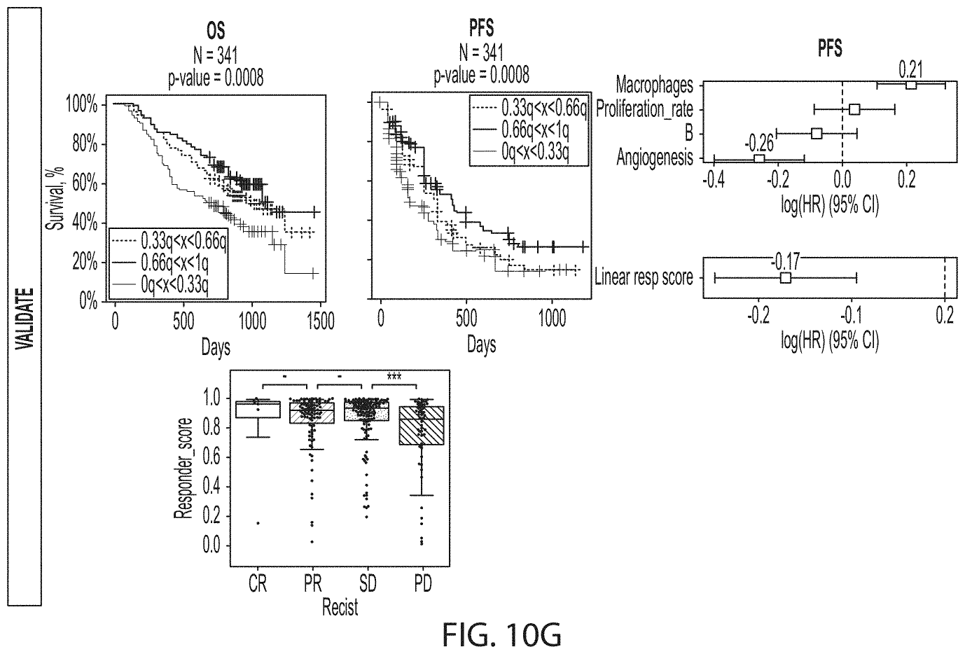

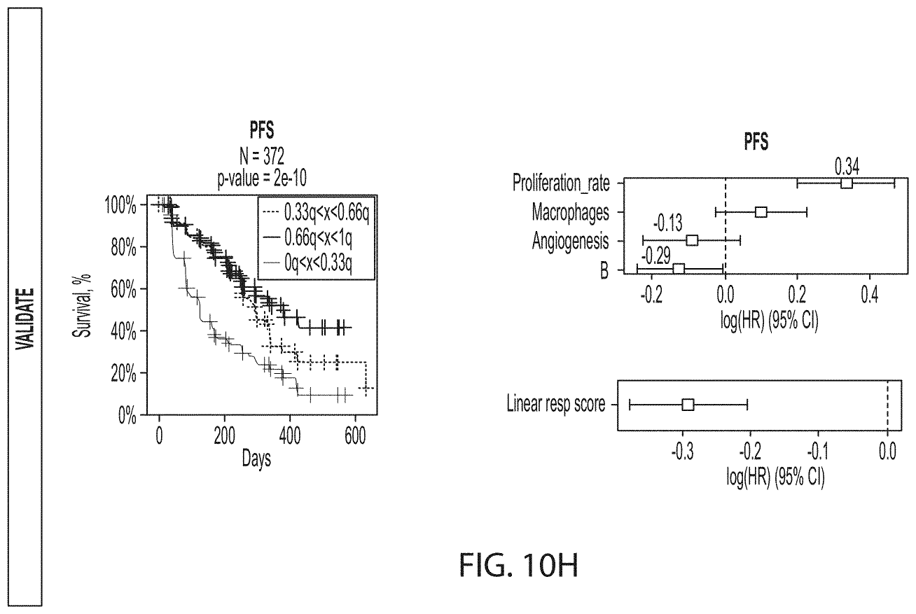

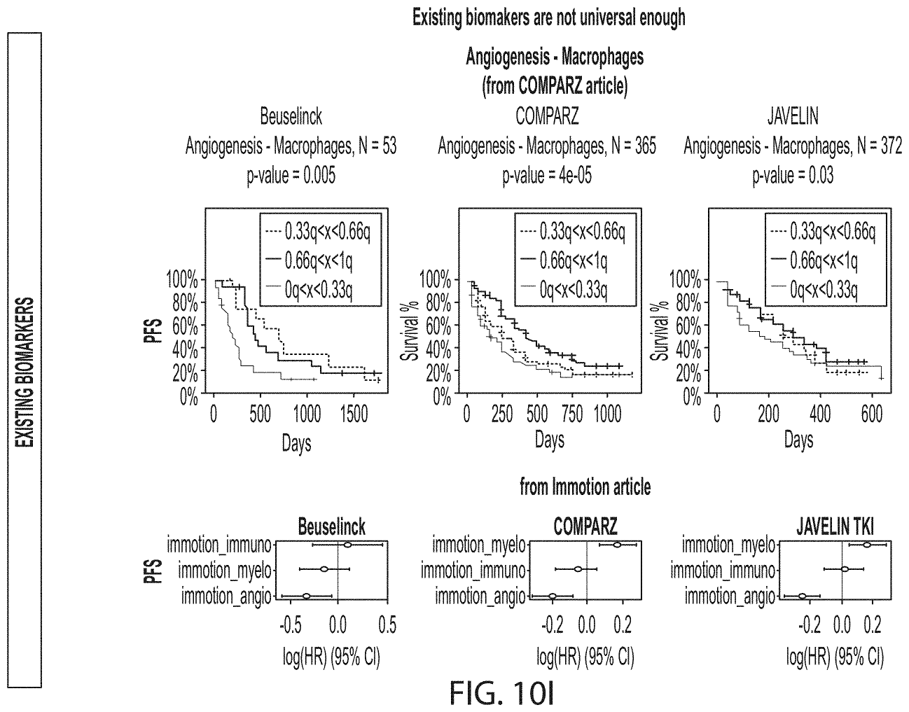

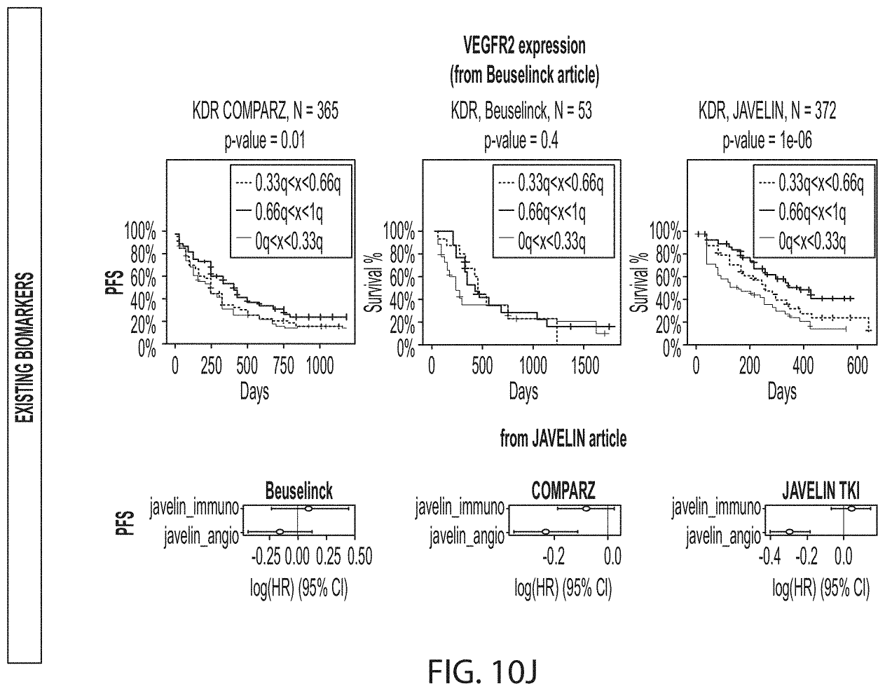

Examples of training and validation of machine learning models for assessing likelihood of response to TKI are shown in FIGS. 9 and 10A-10J. A logistic regression model from the scikit-learn package with default parameters was used. The model was trained on four transcriptomic features: Macrophages signature, Angiogenesis signature and Proliferation rate signature. Similarity of a sample to RC TME type B was also used. Similarity of a sample to a particular RC TME type was calculated as the Spearman correlation coefficient between sample's ssGSEA scores of 33 gene signatures (e.g., based on the gene groups shown in Table 1) and a particular RC TME type's averaged ssGSEA scores of 33 gene signatures (based on the gene groups shown in Table 1). The resulting model score (e.g., responder score) varied from 0 to 1.

The TKI model was trained on samples treated with TKI regimes from two cohorts: MUSMRCC (37 patients PAZ/SUN/CABO/AXI), Beuselinck (53 patients SUN). For model training, patients were from these two cohorts with CR+PR (45 patients) and PD (13 patients) RECIST. These patients were used as two groups for a model to predict. The model was validated on the JAVELIN cohort (372 patients SUN) and COMPARZ cohort (341 patients PAZ/SUN).

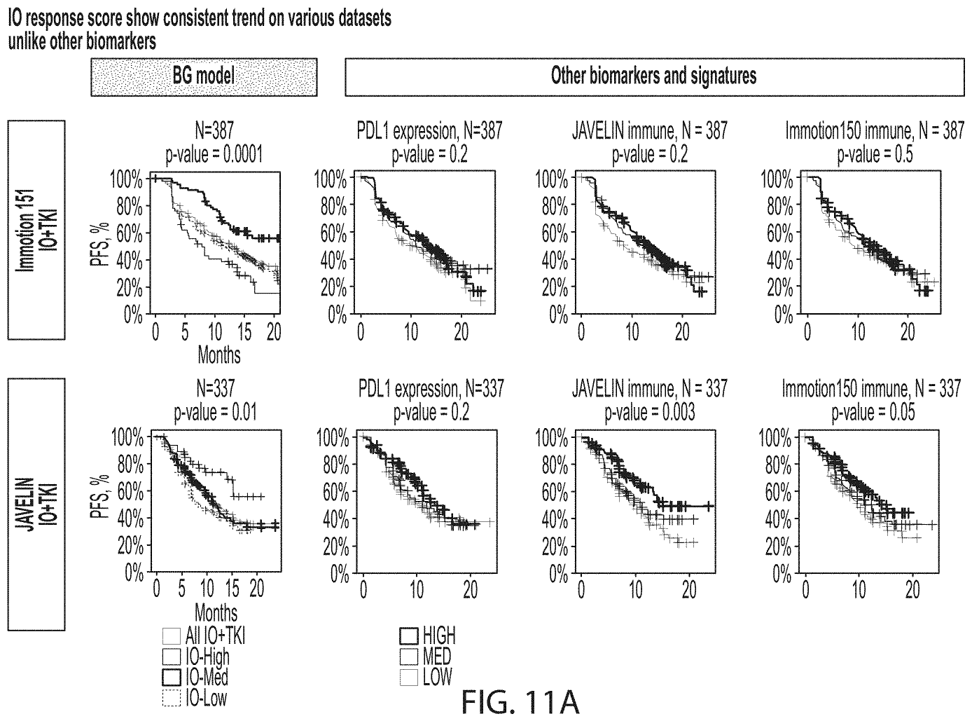

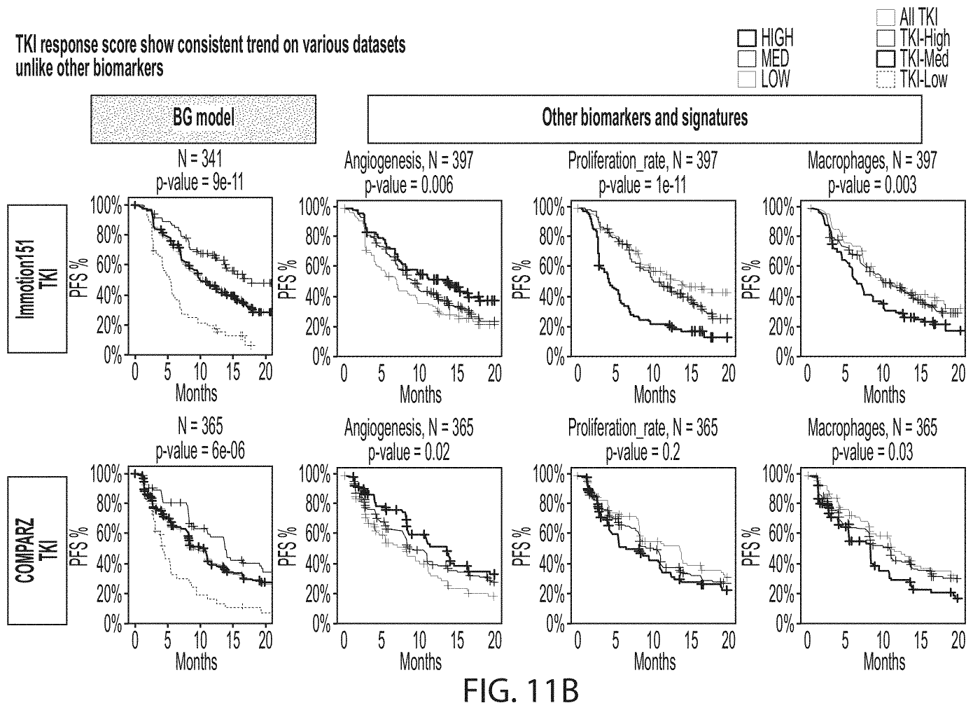

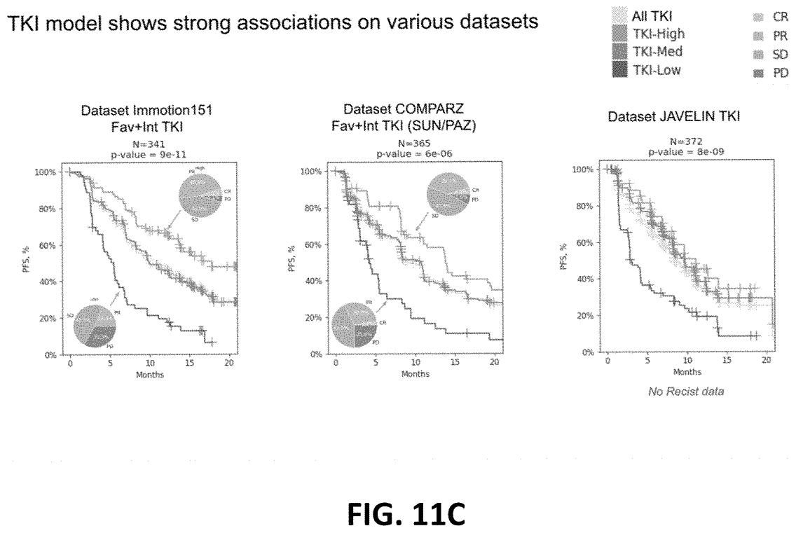

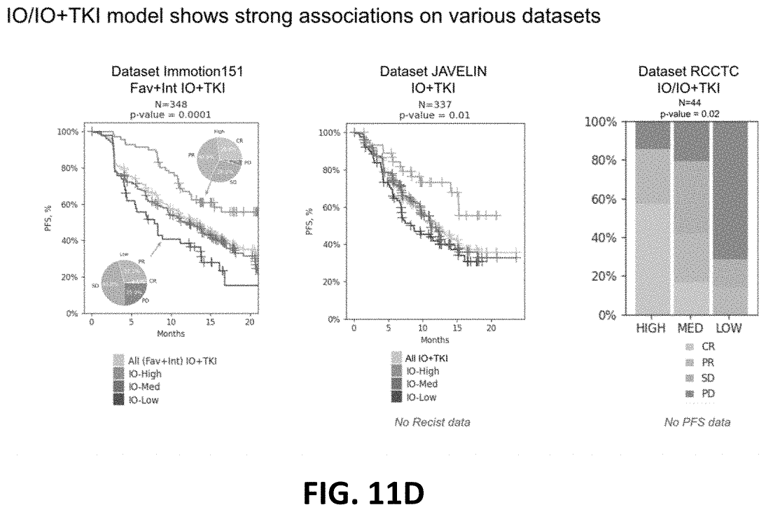

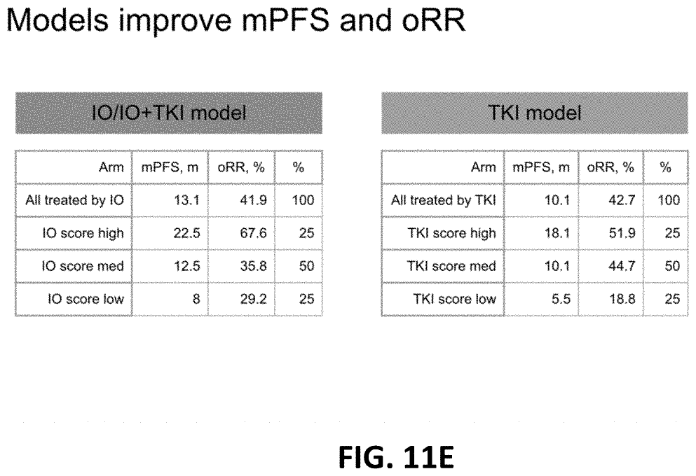

FIGS. 11A-11E provide representative data indicating that IO responder scores and TKI responder scores using machine learning algorithms described by these Examples are more consistent across data sets than previously used classification methods.

### Example 4

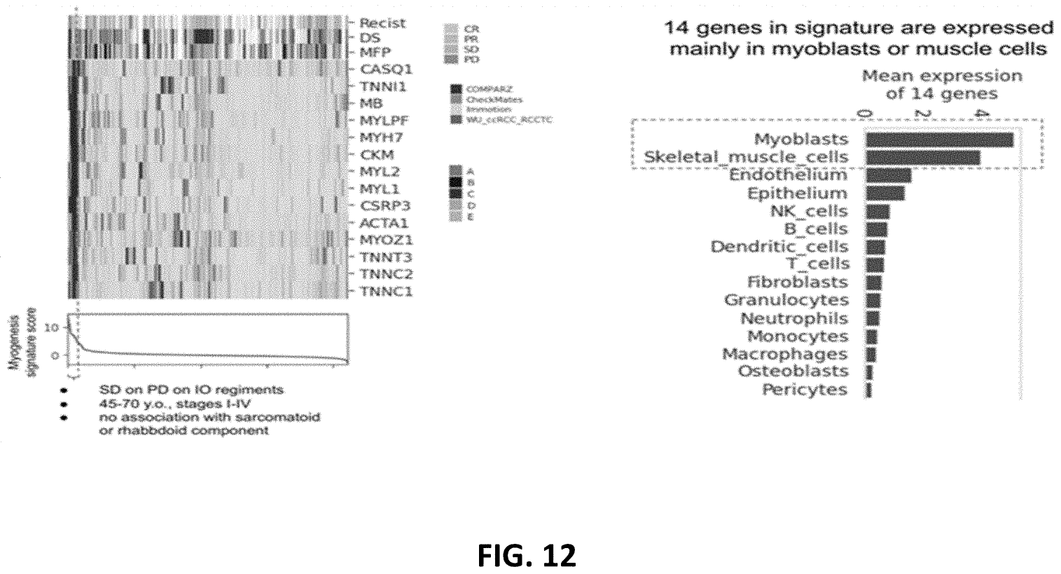

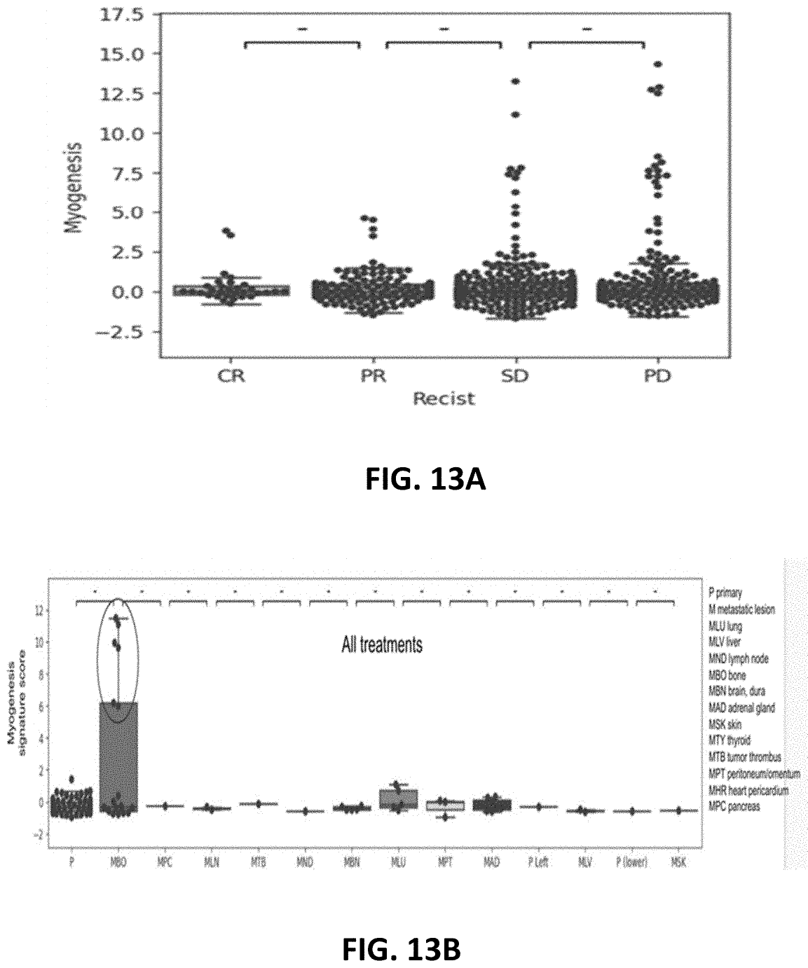

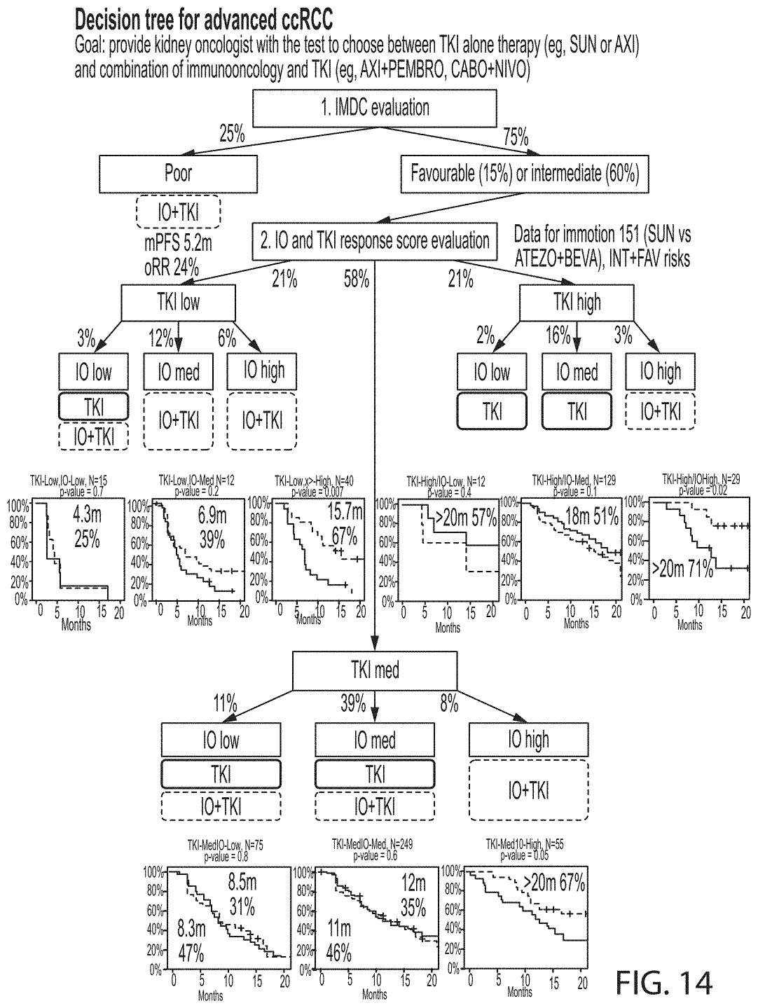

It was observed that a small group of “IO non-responder” patients exhibit an extremely high activity of a Myogenesis signature, which consists of 14 genes (listed in Table 2) that are expressed mainly in myoblasts and skeletal muscle cells. A Myogenesis signature was calculated using the gene group signature pipeline described above: ssGSEA scores were calculated, and the scores inside the cohorts were median scaled. FIG. 12 provides a representative heatmap showing production of a Myogenesis signature for RC (e.g., clear cell renal carcinoma) samples based on ssGSEA analysis and median scaling of 14 genes. This Myogenesis signature was not used for RC TME type identification but was used to identify IO “non-responders” in the IO model described in Example 3. FIG. 13A provides representative data showing RECIST characterization complete response (CR), partial response (PR), stable disease (SD), and progressive disease (PD) plotted against Myogenesis signature. Samples having high Myogenesis score were observed to come from bone metastasis (MBO) patients (FIG. 13B).

TABLE 2

| Gene Group | Gene Group Genes |
|---|---|
| Myogenesis signature | CASQ1 |
| | TNNI1 |
| | MB |
| | MYLPF |
| | MYH7 |
| | CKM |
| | MYL2 |
| | MYL1 |
| | CSRP3 |
| | ACTA1 |
| | MYOZ1 |
| | TNNT3 |
| | TNNC2 |
| | TNNC1 |

TABLE 3
Representative NCBI Accession Numbers for genes listed in Table 1

| gene name | mRNA Accession |
|---|---|
| AADAC | XM_005247104; NM_001086 |
| ACLY | NM_198830; NM_001303275; XM_017024688; XM_005257395; NM_001096; |
| | NM_001303274 |
| ACO1 | NM_002197; XM_011517888; NM_001278352; NM_001362840 |
| ACO2 | XM_017028812; NM_001098; XM_024452250 |
| ACTA2 | NM_001141945; NM_001320855; NM_001613 |
| ACYP1 | NR_126393; NR_126394; NM_203488; NM_001107; NM_001302616; |
| | NM_001302617; NR_126395 |
| ACYP2 | NM_138448; NM_001320589; NM_001320587; NM_001320588; NM_001320590; |
| | NM_001320586 |
| ADAM8 | NM_001164490; NM_001164489; XM_017015466; XM_011539117; NM_001109; |
| | XM_017015465 |
| ADARB2 | NM_018702 |
| ADCY1 | NM_001281768; XM_005249585; NM_021116; XM_005249584 |
| ADCY2 | XR_427657; XR_001741974; XM_011513942; NM_020546; XR_001741973 |
| ADCY3 | XM_011532494; XM_017003188; NM_001320613; NM_001377130; |
| | XM_006711925; XM_011532496; XM_017003189; NM_004036; XM_011532493; |
| | XM_011532492; NM_001377128; NM_001377131; XM_011532490; |
| | XM_017003192; NM_001377129; NM_001377132; XM_011532489; |
| | XM_011532491; XM_017003187; XM_017003191 |
| ADCY5 | XM_017005638; XM_011512360; XM_011512361; XM_006713483; |
| | XM_006713484; XM_011512359; XM_005247078; NM_001199642; |
| | NM_001378259; XM_017005639; NM_183357 |
| ADCY6 | NM_001390831; NM_015270; XM_017018743; XR_001748565; NM_001390830; |
| | XM_006719210; NM_020983 |
| ADCY7 | NM_001114; XM_011522842; XM_011522839; XM_017022896; XM_017022899; |
| | XM_011522837; XM_011522840; XM_011522835; XM_011522836; |
| | XM_017022897; XR_001751822; NM_001286057; XM_017022898; |
| | XM_011522838 |
| ADCY8 | XM_017013006; XM_005250769; XM_017013007; XM_006716501; NM_001115 |
| ADCY9 | XM_011522353; XM_005255079; NM_001116 |
| ADSL | XM_011529977; XM_017028636; XM_017028640; XM_024452166; |
| | XM_017028639; NM_000026; NR_134256; NM_001317923; XR_002958670; |
| | NM_001123378; XR_001755176; NM_001363840; XM_017028637; |
| | XM_017028638; XR_002958671; XM_011529980; XR_937825 |
| ALDH3A1 | NM_000691; XM_005256524; XM_011523731; NM_001135167; NM_001330150; |
| | NM_001135168; XM_005256522; XM_005256523 |
| ALDH1B1 | XM_011517802; NM_000692 |
| ALDH3A2 | NM_001369138; NM_001369148; XM_024450651; NM_000382; NM_001369139; |
| | NM_001369136; NM_001369137; NM_001031806; NM_001369146 |
| ALDOA | NM_184043; NM_001127617; NM_184041; NM_001243175; NM_001355562; |
| | NM_001355565; NM_001355563; NM_001355564; NM_001243177; NM_000034 |
| ALDOB | NM_000035 |
| ALDOC | XM_005257949; NM_005165; XM_011524556 |
| ANGPT1 | NM_001314051; NM_001146; NM_001199859; NM_139290; XR_928319 |
| ANGPT2 | NM_001118888; NM_001386335; NM_001386337; NM_001118887; NM_001147; |
| | NM_001386336 |
| ARG1 | NM_001369020; NM_000045; NM_001244438; NR_160934 |
| ASTN1 | NM_001364856; NM_004319; XR_001737193; NM_207108; XM_017001341; |
| | XR_001737194; NM_001286164; XR_921796 |
| B2M | XR_002957658; XM_005254549; NM_004048 |
| ADGRB1 | XM_017013695; NM_001391987; XM_017013693; NM_001391985; |
| | NM_001391986; NM_001391988; XM_017013692; XM_017013696; |
| | XM_017013697; XM_017013691; NM_001702; XM_017013694; XM_011517202 |
| CCND1 | NM_053056 |
| TNFRSF17 | NM_001192 |
| BPGM | XM_011516527; NM_199186; NM_001293085; NM_001724 |
| BUB1 | NM_004336; XR_923001; NM_001278617; NM_001278616 |
| CA9 | XR_428428; NM_001216; XR_001746374 |
| SLC25A20 | NM_000387 |
| CBR1 | NM_001286789; NM_001757 |
| CCNB1 | NM_001354844; NM_031966; NM_001354845 |
| CCNE1 | XM_011527440; NM_001238; NM_001322259; NM_001322261; NM_001322262; |
| | NM_057182 |
| CD3D | NM_001040651; NM_000732 |
| CD3E | NM_000733; |
| CD3G | XM_005271724; XM_006718941; NM_000073 |
| CD5 | NM_014207; NM_001346456 |
| CD8A | NM_001145873; NM_001382698; NM_001768; NR_168478; NR_168479; |
| | NM_171827; NR_168480; NR_168481; NR_027353 |
| CD8B | NM_172102; NM_172100; NM_001178100; NM_004931; NM_172101; |
| | NM_172213; NM_172099; XM_011533164 |
| CD19 | NM_001178098; NM_001385732; NM_001770; XR_950871; XM_006721103; |
| | NR_169755; XM_011545981 |
| MS4A1 | NM_021950; NM_152866; NM_152867; |
| CD22 | NM_001185100; NM_001185099; NM_024916; NM_001185101; NM_001771; |
| | NM_001278417 |
| CD27 | XM_017020232; XM_017020233; NM_001242; XM_011521042; XM_017020234 |
| CD28 | XM_011512195; NM_006139; XM_011512197; NM_001243078; NM_001243077; |
| | XM_011512194 |
| CD80 | NM_005191 |
| CD86 | NM_001206924; NM_006889; NM_176892; NM_001206925; NM_175862; |
| CD38 | NM_001775; NR_132660 |
| CD40 | NM_001302753; NM_001322422; NM_152854; NM_001322421; NM_001362758; |
| | NM_001250; NR_136327; XM_011529109; XM_005260619; XM_017028135; |
| | XM_017028136; NR_126502 |
| CD40LG | NM_000074 |
| CD68 | NM_001251; NM_001040059 |
| CD70 | NM_001252; NM_001330332 |
| CD79A | NM_021601; NM_001783 |
| CD79B | NM_001039933; NM_021602; NM_000626; NM_001329050 |
| CDH2 | XM_011525788; NM_001308176; XM_017025514; NM_001792 |
| CDH5 | NM_001114117; XM_024450133; NM_001795; XM_011522801 |
| CDH17 | NM_004063; XM_011516790; NM_001144663 |
| CDK2 | ; NM_001290230; XM_011537732; NM_052827; NM_001798 |
| CETN3 | NM_004365; NM_001297765; NM_001297768 |
| CCR3 | NM_001164680; NM_001837; XM_017005686; XM_011533335; NM_178328; |
| | NM_178329; XM_017005685; XM_006712960 |
| CCR4 | XM_017005687; NM_005508 |
| CCR8 | NM_005201 |
| CMKLR1 | NM_001142343; NM_001142345; XM_017018820; NM_001142344; NM_004072 |
| ABCC2 | XM_011539291; XR_945605; XM_006717631; XM_017015675; XR_945604; |
| | NM_000392; XM_006717630 |
| COL1A1 | XM_005257058; XM_005257059; XM_011524341; NM_000088 |
| COL1A2 | NM_000089 |
| COL3A1 | NM_000090; NM_001376916 |
| COL4A1 | NM_001845; XM_011521048; NM_001303110 |
| COL5A1 | XM_017014266; XR_001746183; NM_000093; NM_001278074 |
| COL6A1 | NM_001848 |
| COL6A2 | NM_001849; NM_058175; NM_058174 |
| COL6A3 | NM_057164; NM_057167; NM_057166; NM_004369; NM_057165 |
| COL11A1 | XM_017000337; XM_017000335; XM_017000336; NR_134980; NM_080629; |
| | XM_017000334; NM_001190709; NM_001854; NM_080630 |
| CR2 | NM_001877; NM_001006658; XM_011509206 |
| CS | NM_004077; NM_198324 |
| CSF1 | NM_000757; NM_172210; XM_017000369; NM_172211; NM_172212 |
| CSF1R | NM_001375320; NM_005211; NR_164679; NM_001349736; NM_001288705; |
| | NM_001375321; NR_109969 |
| CSF2 | NM_000758 |
| CSF2RA | NM_001379164; NM_001379166; XM_011545623; NM_001161530; |
| | NM_001379165; NM_006140; NM_172247; NM_172248; XM_011545627; |
| | NM_001161531; NM_001161532; NM_001379168; NM_172245; NM_001161529; |
| | NM_001379158; NM_001379161; NM_001379162; NM_172246; XM_011545620; |
| | XM_011546165; XM_011546167; XM_011546169; XM_011546170; |
| | NM_001379155; NM_001379163; NM_172249; XM_011545622; NM_001379160; |
| | NM_001379167; NR_027760; XM_011545618; XM_011545628; XM_011546174; |
| | NM_001379153; XM_011546175; NM_001379154; NM_001379156; |
| | NM_001379159; NM_001379169 |
| CSF3 | NR_168489; NR_168491; NM_000759; NM_001178147; NM_172219; |
| | NM_172220; NR_168490; NR_033662 |
| CSF3R | NM_000760; XM_005270493; XM_011540750; NM_156039; XM_011540749; |
| | NM_156038; XM_017000370; NM_172313; XM_011540748 |
| SLC25A10 | NM_001270888; XM_017024220; NM_001270953; NM_012140 |
| CTLA4 | NM_001037631; NM_005214 |
| CTSK | NM_000396 |
| CTSW | NM_001335 |
| CX3CR1 | NM_001171174; NM_001337; NM_001171171; NM_001171172 |
| CYP17A1 | NM_000102 |
| AKR1C1 | XM_017015791; NM_001353 |
| AKR1C2 | NM_001354; NM_001321027; NM_001135241; NM_001393392; NM_205845 |
| NQO1 | NM_001286137; NM_001025434; NM_001025433; NM_000903 |
| DLAT | NM_001372032; NM_001372034; NM_001372036; NM_001372038; |
| | NM_001372031; NM_001372035; NM_001372039; NM_001372042; NM_001931; |
| | NR_164072; NM_001372033; NM_001372037; NM_001372040; NM_001372041 |
| DLD | NM_001289751; NM_000108; NM_001289750; NM_001289752 |
| DLST | NM_001244883; XR_001750184; NM_001933; NR_033814; NR_045209 |
| E2F1 | NM_005225 |
| ECH1 | XM_024451408; XM_024451409; XM_017026448; NM_001398 |
| ELN | XM_011515869; XM_011515873; XM_017011814; XM_005250187; |
| | XM_011515871; XM_011515872; NM_001278914; XM_005250188; |
| | XM_011515874; NM_000501; NM_001278912; NM_001278939; XM_011515877; |
| | NM_001081753; XM_011515876; NM_001081754; NM_001278917; |
| | XM_017011813; NM_001278915; NM_001278918; XM_011515868; |
| | XM_011515870; NM_001081755; NM_001278916; XM_011515875; |
| | NM_001081752; NM_001278913 |
| ENG | NM_000118; NM_001114753; NM_001278138 |
| ENO1 | NM_001201483; NM_001353346; NM_001428 |
| ENO2 | NM_001975 |
| ENO3 | NM_001374524; NM_001193503; NM_001374523; NM_053013; NM_001976; |
| | XM_011523729 |
| EPHA5 | XM_005265653; XM_017007880; XM_017007881; NM_001318761; |
| | XM_017007879; NM_001281765; XM_011531735; NM_001281767; NM_004439; |
| | XM_017007878; NM_001281766; NM_182472 |
| ETFB | XM_024451418; NM_001985; NM_001014763 |
| ACSL1 | XM_017007887; NR_167706; NM_001381877; NM_001381878; NM_001381883; |
| | NM_001381885; NM_001381890; NR_167698; NR_167702; NR_167704; |
| | NM_001286708; NM_001381882; NM_001286710; NM_001381881; |
| | NM_001381886; NM_001381888; NM_001381879; NM_001381887; NM_001995; |
| | NM_001286712; NM_001381880; NM_001381889; NR_167708; NR_167709; |
| | NR_167703; NR_167705; NM_001286711; NM_001381884 |
| ACSL3 | NM_001354158; NM_203372; NM_004457; NM_001354159 |
| ACSL4 | XM_011530888; XM_024452351; NM_001318510; NM_022977; XM_005262109; |
| | XM_006724635; XM_011530889; NM_001318509; NM_004458 |
| FAH | NM_001374377; NM_001374380; NM_000137 |
| FBLN1 | NM_006485; NM_006486; NM_001996; NM_006487 |
| FBP1 | XM_006717005; NM_001127628; NM_000507 |
| FCGR3B | NM_001271036; NM_001271037; NM_000570; NM_001244753; NM_001271035 |
| FECH | XM_011525881; NM_001371095; XM_011525882; NM_000140; NM_001012515; |
| | NM_001374778; NM_001371094 |
| FGF2 | NM_001361665; NM_002006 |
| FH | XM_011544132; NM_000143 |
| FLT1 | NM_001160030; NM_001159920; XM_011535014; XM_017020485; |
| | NM_001160031; NM_002019 |
| FN1 | NM_001306129; NM_001365519; NM_212474; NM_001306132; NM_001365517; |
| | NM_001365522; NM_001306131; NM_001365521; NM_212476; NM_212478; |
| | NM_212475; NM_001365523; NM_001365524; NM_002026; NM_001365520; |
| | NM_212482; NM_001365518; NM_054034; NM_001306130 |
| FTH1 | NM_002032 |
| G6PC | NM_000151; NM_001270397 |
| G6PD | NM_001042351; NM_000402; NM_001360016 |
| GAPDH | NM_001256799; NM_001289745; NM_001357943; NR_152150; NM_001289746; |
| | NM_002046; NM_001357938; NM_001357944 |
| GCK | XM_024446707; NM_001354800; NM_001354802; NM_033507; NM_000162; |
| | NM_001354801; NM_001354803; NM_033508 |
| GCLC | XM_017010749; NM_001498; NM_001197115 |
| GCLM | XM_017001057; XM_011541261; XM_017001056; NM_001308253; NM_002061 |
| GOT1 | NM_002079 |
| GOT2 | NM_002080; NM_001286220 |
| GPD2 | NM_001083112; XM_005246469; NM_000408; XM_011510977; XM_024452798; |
| | XM_011510978; XM_017003830 |
| GPI | XM_011526754; NM_001184722; NM_001329910; NM_000175; NM_001289789; |
| | NM_001329909; NM_001329911; NM_001289790 |
| CCR10 | NM_016602 |
| XCR1 | XM_017006197; XM_017006199; XM_017006201; XM_017006198; |
| | NM_001024644; NM_005283; NR_170111; XM_017006200; NM_001381860 |
| CXCR3 | XM_017029435; XM_017029436; NM_001504; NM_001142797; XM_005262256; |
| | XM_005262257 |
| FFAR2 | NM_005306; NM_001370087; XM_017026711 |
| GPX2 | NR_046321; NR_138078; NR_046320; NM_002083 |
| CXCL1 | NM_001511; NR_046035 |
| CXCL2 | NM_002089 |
| GSR | NM_001195103; NM_000637; NM_001195102; NM_001195104; |
| GUCY1A2 | NM_001256424; NM_000855 |
| GUCY1A3 | NM_001379675; NM_001130687; NM_001379672; NM_001379673; |
| | NM_001379676; NM_001256449; NM_001130686; NM_001130683; |
| | NM_001130684; NM_001130685; NM_001379674; XM_005262957; |
| | XM_011531900; NM_001379669; NM_000856; NM_001130682; NM_001379668; |
| | NM_001379671; XM_005262956; NM_001379666; NM_001379667; |
| | NM_001379670 |
| GUCY1B3 | NM_001291955; XM_017008130; NM_001291953; XM_011531901; |
| | NM_001291952; NM_000857; XM_017008132; XM_017008133; NM_001291951; |
| | NM_001291954; XM_017008131 |
| GUCY2C | NM_004963; XM_011520631 |
| GUCY2F | XR_244473; NM_001522 |
| GUCY2D | XM_011523816; NM_000180 |
| HABP2 | NM_004132; NM_001177660 |
| HK1 | NM_001322366; NM_001322367; NM_033498; XM_011539732; NM_000188; |
| | NM_001358263; NM_033497; NM_033500; XM_024447969; NM_033496; |
| | XM_005269737; NM_001322364; NM_001322365 |
| HK2 | NM_000189; XM_005264280; XM_011532807; XM_017003945; NM_001371525 |
| HK3 | XM_011534540; XR_941101; XM_017009411; NM_002115; XR_941102 |
| HLA-DPA1 | NM_001242525; NM_033554; NM_001242524 |
| HLA-DPB1 | NM_002121 |
| HLA-DQA1 | NM_002122; XM_006715079; |
| HLA-DQB1 | NM_001243962; NM_001243961; NM_002123 |
| HLA-DRB1 | XM_024452553; NM_001359194; XR_002958969; NM_001243965; NM_002124; |
| | NM_001359193; XR_002958970 |
| HRG | NM_000412; XM_005247415 |
| IDH1 | NM_001282387; NM_001282386; NM_005896 |
| IDH2 | NM_001290114; NM_002168; NM_001289910 |
| IDH3A | XM_005254336; XM_024449912; NM_005530; XM_024449911 |
| IDH3B | XR_001754267; NM_174855; NM_006899; NM_174856; XR_001754265; |
| | XR_001754266; NM_001330763; NR_136344; NM_001258384 |
| IDH3G | NM_174869; NM_004135 |
| IFNA2 | NM_000605 |
| IFNB1 | NM_002176 |
| JCHAIN | NM_144646 |
| IL1B | NM_000576; XM_017003988 |
| IL2 | NM_000586; |
| IL4 | NM_000589; NM_001354990; NM_172348 |
| IL5 | XM_011543374; XM_005271988; XM_011543373; NM_000879 |
| IL6 | NM_001318095; NM_000600; NM_001371096; XM_011515390; XM_005249745; |
| IL6R | NM_001382771; XM_005245139; NM_001206866; NM_001382770; NM_181359; |
| | NM_001382773; NM_000565; NM_001382769; NM_001382774; XM_005245140; |
| | NM_001382772; XM_017001199; XM_017001201 |
| CXCL8 | NM_000584; NM_001354840 |
| CXCR1 | NM_000634 |
| CXCR2 | XM_017003990; XM_017003992; NM_001168298; NM_001557; XM_005246530; |
| | XM_017003991 |
| IL10 | NM_000572; NR_168467; NR_168466; NM_001382624 |
| IL12A | NM_000882; NM_001354583; NM_001354582; NM_001397992 |
| IL12B | NM_002187 |
| IL12RB2 | NR_047584; XM_011541384; XM_005270827; XM_006710617; NM_001374259; |
| | XM_011541383; NM_001258215; NM_001258216; XM_017001204; |
| | NM_001258214; NM_001319233; XM_005270828; XM_017001203; NM_001559; |
| | NR_047583 |
| IL13 | NM_001354991; NM_001354992; NM_002188; NM_001354993 |
| TNFRSF9 | NM_001561; XM_006710618 |
| IDO1 | NM_002164 |
| CXCL10 | NM_001565; NR_168520 |
| IREB2 | NM_001320942; NM_001320943; NM_004136; NM_001320941; NM_001354994 |
| IRF4 | NM_001195286; NR_046000; NR_036585; XM_006715090; NM_002460 |
| IRF5 | NM_001242452; XM_006715974; NM_001364314; NM_032643; XM_011516158; |
| | XM_011516160; NM_001347928; NM_001098629; XM_011516159; |
| | NM_001098627; NM_001098630 |
| KCNF1 | NM_002236 |
| KEL | NM_000420; XM_005249994; XM_005249993 |
| KIR2DL4 | NM_001080770; NM_001080772; NM_002255; NM_001258383 |
| KLRC2 | NM_002260 |
| LAG3 | NM_002286; XM_011520956 |
| LAMA3 | XM_011525981; XM_017025743; XR_001753199; NM_001127717; NM_000227; |
| | XM_011525978; XM_011525979; NM_198129; XM_011525980; XM_017025744; |
| | XM_011525982; NM_001302996; NR_130106; NM_001127718 |
| LAMB3 | XM_005273124; NM_001127641; XM_017001272; NM_000228; NM_001017402 |
| LAMC2 | NM_005562; NM_018891; XM_017001273 |
| LDHA | NR_028500; NM_001165414; NM_001165415; NM_001165416; NM_005566; |
| | NM_001135239 |
| LDHC | NM_002301; NM_017448; XM_017017721; XM_017017722 |
| LGALS7 | NM_002307 |
| LGALS9 | XM_011524796; NM_001330163; NR_024043; XM_006721893; XM_006721895; |
| | NM_002308; XM_006721892; XM_017024623; NM_009587 |
| LIPC | XM_017022176; XM_005254374; XM_006720502; XM_024449916; |
| | XM_005254372; NM_000236; XM_024449917 |
| LRP1 | NM_002332 |
| LTB | NM_002341; NM_009588 |
| CYP4F3 | XM_005259911; NM_000896; NM_001199209; NM_001199208; XM_024451515; |
| | NM_001369696; XM_011528014; XM_017026815 |
| MAFG | NM_002359; NM_032711 |
| MCM2 | NM_004526; XM_024453531; NR_073375 |
| MCM6 | NM_005915 |
| MDH1 | NM_001316374; NM_001199111; NM_001199112; NM_005917 |
| MDH2 | NM_005918; NM_001282403; NR_104165; NM_001282404 |
| ME1 | XM_011535836; NM_002395 |
| ME2 | NM_002396; NR_174094; XR_935223; NM_001168335 |
| KITLG | NM_003994; NM_000899 |
| CXCL9 | NM_002416 |
| MKI67 | NM_002417; NM_001145966; XM_006717864; XM_011539818 |
| MMP1 | NM_001145938; NM_002421; |
| MMP2 | NM_001302509; NM_001127891; NM_001302508; NM_001302510; NM_004530 |
| MMP3 | NM_002422 |
| MMP7 | NM_002423 |
| MMP9 | NM_004994; |
| MMP11 | NM_005940; NR_133013 |
| MMP12 | NM_002426; |
| MRC1 | NM_002438; NM_001009567 |
| MSR1 | NM_138716; NM_002445; XM_024447161; NM_138715; NM_001363744 |
| MUC1 | NM_001018017; NM_001044391; NM_001044393; NM_001204291; |
| | NM_001044390; NM_001204285; NM_182741; NM_001371720; NM_001204289; |
| | NM_001204290; NM_001204293; NM_001018016; NM_001044392; |
| | NM_001204286; NM_001204287; NM_001204288; NM_001204295; |
| | NM_001018021; NM_001204292; NM_001204294; NM_001204297; |
| | NM_001204296; NM_002456 |
| MYBL2 | NM_001278610; NM_002466 |
| MYO7B | XM_017004169; XR_001738748; NM_001393594; XM_011511218; NM_183371; |
| | NM_001393586; XR_001738749; NM_001080527 |
| NKG7 | XM_006723228; XM_005258955; NM_001363693; NM_005601 |
| NOS2 | NM_153292; NM_000625 |
| NOS3 | NM_001160110; NM_000603; NM_001160109; NM_001160111 |
| NPR1 | XM_017001374; XM_005245218; NM_000906 |
| NPR2 | XM_024447560; NM_003995; XM_024447556; XM_024447557; XM_024447559; |
| | NM_001378923; NM_000907; XM_024447558; XM_024447561 |
| OGDH | XM_024446783; NM_001003941; XM_005249759; NM_001363523; |
| | XM_011515408; NM_001165036; NM_002541 |
| OSM | NM_001319108; NM_020530 |
| PRDX1 | NM_002574; NM_001202431; NM_181697; NM_181696 |
| SERPINE1 | NM_000602; NM_001386463; NM_001386465; NM_001386457; NM_001386466; |
| | NM_001165413; NM_001386456; NM_001386460; NM_001386461; |
| | NM_001386462; NM_001386459; NM_001386464; NM_001386458 |
| PAX5 | NM_001280547; NM_001280553; NM_016734; NM_001280548; NR_103999; |
| | NM_001280551; NM_001280555; NM_001280554; NM_001280552; |
| | NM_001280556; NM_001280549; NM_001280550; NR_104000 |
| PC | XM_011545086; NM_022172; XM_017017869; XM_005274031; XM_017017868; |
| | XM_006718578; XM_017017871; XM_006718579; NM_000920; XM_005274032; |
| | XM_011545087; XM_017017872; XM_017017870; NM_001040716 |
| PCCA | XM_017020609; XM_017020613; XM_017020616; NM_001178004; NR_148030; |
| | XM_017020611; XR_001749567; XR_001749568; XR_001749569; |
| | NM_001352606; NM_001352610; NM_001352611; NM_001352605; NR_148028; |
| | XM_017020615; NM_001352607; NM_001352609; XM_017020607; |
| | XR_001749574; XR_931615; NR_148029; XM_011521093; XM_017020605; |
| | NM_001352608; NM_001352612; XM_017020606; XR_001749577; NR_148027; |
| | XM_017020612; XR_001749576; NM_000282; NM_001127692; NR_148031 |
| PCCB | NM_000532; XM_011512873; NM_001178014 |
| PCK1 | NM_002591; XM_024451888 |
| PCK2 | NM_001291556; NM_001018073; XM_006720158; NM_001308054; NM_004563 |
| PDCD1 | XM_017004293; NM_005018; XM_006712573 |
| PDE1A | NM_001258312; NM_005019; NM_001258314; NM_001395258; NM_001395263; |
| | XM_011511324; NM_001395260; NM_001395262; NM_001395266; |
| | XM_011511325; XM_017004300; XM_017004301; NM_001003683; |
| | NM_001395259; NM_001395264; NM_001395265; NM_001395268; |
| | NM_001395269; XR_002959304; NM_001363871; NM_001395267; |
| | XM_011511323; XM_011511326; XM_017004294; XR_001738769; |
| | NM_001258313; NM_001395261 |
| PDE1C | XR_001744804; XR_001744805; XR_001744806; NM_001191057; |
| | NM_001322059; XR_001744802; XR_001744803; NM_001191056; |
| | NM_001322058; NM_001322056; XM_017012264; XM_017012265; |
| | XM_017012266; XR_002956451; NM_001322057; NM_005020; XM_017012267; |
| | NM_001191058; NM_001191059; NM_001322055 |
| PDE2A | NM_001143839; NM_001243784; XM_005274040; NM_001146209; NM_002599 |
| PDE3A | NM_001378409; NM_001378407; NM_000921; XM_017019421; NM_001244683; |
| | NM_001378408; XM_017019420 |
| PDE3B | XR_001747903; NM_000922; NM_001363570; XM_017017912; XM_006718249; |
| | XM_017017911; NM_001363569 |
| PDE4A | NM_001111307; NM_001243121; XM_017026865; NM_001111309; |
| | XM_024451534; XM_011528055; NM_006202; XM_011528054; NM_001111308 |
| PDE4B | NM_001297441; NM_001037341; NM_001037339; NM_002600; XM_017001445; |
| | NM_001297440; NM_001297442; XM_005270924; XM_005270925; |
| | XM_006710680; NM_001037340 |
| PDE4C | NM_001330172; NM_001369701; XM_011528058; NM_001098819; |
| | XM_024451535; NR_040546; NM_000923; NM_001098818; NM_001395274 |
| PDE4D | NM_001364602; XM_017009565; XM_017009567; XM_024446110; |
| | NM_001104631; NM_001197218; NM_001197219; NM_001197221; |
| | NM_001197223; NM_001349241; XM_011543469; NM_001364601; |
| | NM_001364603; XM_011543470; XM_024446112; NM_001197220; |
| | NM_001349242; NM_001364604; XM_017009566; NM_001165899; |
| | NM_001197222; NM_001364599; XM_011543471; NM_006203; XM_011543473; |
| | NM_001349243; NM_001364600 |
| PDE6A | XM_011537653; XM_017009572; XM_011537651; XM_011537652; |
| | XM_011537654; NM_000440; XM_011537650 |
| PDE6C | NM_006204 |
| PDE6D | NR_110994; XM_011511342; NM_001291018; NM_002601 |
| PDE6G | NM_001365725; NR_026872; NM_001365724; NM_002602; XM_017024736; |
| | NR_158591 |
| PDE6H | NM_006205; XM_017019431; |
| PDE7A | NM_002603; NM_001242318; NM_002604; XM_011517540; XM_017013538 |
| PDE8A | XM_024449946; XR_001751313; NM_173454; XM_011521668; XM_024449947; |
| | XM_017022309; NM_001243137; NM_002605; NM_173455; NM_173457; |
| | XM_017022308; XM_017022311; NM_173456; XM_017022310 |
| PDE9A | XM_024452084; NM_001001571; NM_001001578; NM_001001580; |
| | NM_001001585; XM_011529600; NM_001001569; NM_001315533; NM_002606; |
| | XM_017028367; NM_001001574; NM_001001576; NM_001001567; |
| | NM_001001568; XM_011529598; NM_001001572; NM_001001582; |
| | NM_001001570; NM_001001573; NM_001001575; NM_001001579; |
| | NM_001001581; XM_017028366; NM_001001577; NM_001001583; |
| | NM_001001584 |
| PDE1B | NM_001288769; NM_001315534; XM_011538456; XM_017019433; |
| | NM_001165975; NM_001288768; XM_017019432; NM_001315535; NM_000924 |
| PDE6B | XM_011513474; NM_001145291; NM_001379246; NM_001379247; |
| | NM_001145292; NM_001350154; XM_011513473; XM_011513476; |
| | XM_011513478; XM_017008284; XM_011513475; NM_000283; NM_001350155 |
| PDHA1 | XM_017029574; NM_001173454; NM_001173455; NM_001173456; NM_000284 |
| PDHA2 | NM_005390 |
| PDHB | NM_001315536; NR_033384; NM_001173468; NM_000925 |
| PFKFB1 | NM_001271804; XM_017029578; XM_017029576; NM_002625; NR_073450; |
| | XM_024452389; XM_017029577; NM_001271805 |
| PFKFB2 | XM_024447654; XM_024447655; XM_024447657; NM_001018053; |
| | XM_005273162; XM_024447656; NM_006212 |
| PFKFB3 | NM_001282630; XM_005252464; XM_017016326; XM_017016329; NM_004566; |
| | NM_001363545; XM_011519493; NR_136554; XM_017016328; NM_001145443; |
| | NM_001323016; NM_001323017; XM_024448037; XM_017016327; |
| | NM_001314063 |
| PFKFB4 | XM_017006617; XM_024453595; NM_001317136; XM_017006614; |
| | XM_017006616; NM_001317137; XM_011533829; NM_001317134; |
| | XM_017006615; NM_001317135; NM_001317138; NM_004567 |
| PFKL | XM_006724011; XM_011529603; XM_017028368; NM_001002021; NM_002626; |
| | XM_017028369; XM_017028372; XM_024452085; XM_005261135; |
| | XM_005261137; XM_017028370 |
| PFKM | XM_011538487; XM_024449020; NM_000289; NM_001354739; NR_148954; |
| | NR_148959; NM_001354748; XM_017019469; XM_024449021; XM_005268976; |
| | NM_001166686; NM_001354741; NM_001354744; NR_148957; XM_005268974; |
| | XM_024449022; NM_001354743; NM_001363619; NM_001166687; |
| | NM_001166688; NM_001354736; NM_001354745; NR 148956; NR 148958; |
| | XM_005268979; NM_001354737; NM_001354738; NM_001354746; |
| | NM_001354735; NM_001354740; NM_001354742; NM_001354747; NR_148955 |
| PFKP | NM_001323073; XM_005252466; XM_006717449; NM_001323067; |
| | XM_005252465; NM_001323070; NM_001345944; NM_002627; XM_024448038; |
| | NM_001323071; NM_001323072; NM_001323069; NM_001242339; |
| | NM_001323068; NM_001323074 |
| PFN2 | NM_002628; NM_053024 |
| PGAM1 | NM_001317079; NM_002629 |
| PGAM2 | NM_000290 |
| PGD | NM_001304452; NM_002631; NM_001304451 |
| PGK1 | NM_000291 |
| PGK2 | NM_138733 |
| PGM1 | NM_001172819; NM_001172818; NM_002633 |
| PKLR | XM_017001493; NM_000298; XM_011509640; XM_006711386; NM_181871 |
| PKM2 | XM_006720570; NM_001206797; NM_001206798; XM_011521670; |
| | NM_001206799; NM_002654; XR_001751314; NM_001206796; NM_182470; |
| | NM_182471; XM_005254443; XM_005254445; XM_017022313; NM_001316318 |
| PLK1 | NM_005030 |
| PLOD2 | XM_017006625; XM_024453599; NM_000935; NM_182943; XR_001740176; |
| | XM_005247535; |
| POU2AF1 | XM_006718860; XM_017017932; XM_006718859; XM_005271593; |
| | XM_005271594; NM_006235 |
| PRF1 | NM_005041; NM_001083116 |
| PRTN3 | XM_011528136; NM_002777 |
| PTGS2 | NM_000963; |
| RIT1 | NM_001256820; NM_006912; NM_001256821 |
| CCL1 | NM_002981 |
| CCL2 | NM_002982 |
| CCL3 | NR_168496; NR_168495; NM_002983; NR_168494 |
| CCL5 | NM_001278736; NM_002985 |
| CCL7 | NM_006273 |
| CCL8 | NM_005623 |
| CCL11 | NM_002986 |
| CCL15 | NM_004167; NM_032965; NM_032964 |
| CCL17 | XM_011523256; NM_002987; XM_017023530 |
| CCL22 | NM_002990 |
| CXCL11 | NM_001302123; NM_005409 |
| CXCL5 | NM_002994 |
| XCL1 | XM_011509865; NM_002995 |
| CX3CL1 | NM_001304392; NM_002996 |
| CXCL12 | NM_000609; XR_001747171; XR_001747172; NM_001277990; NM_199168; |
| | XR_001747174; NM_001178134; XR_001747173; NM_001033886 |
| SDHA | XM_024446143; XM_011514072; NM_001330758; XM_011514073; |
| | XM_017009685; XR_002956167; NM_001294332; NM_004168 |
| SDHB | NM_003000 |
| SDHC | NM_001035513; NM_003001; NM_001035511; NM_001035512; NM_001278172; |
| | NR_103459 |
| SDHD | NM_001276504; NM_003002; NM_001276503; NR_077060; NM_001276506 |
| PMEL | NM_001200054; NM_001200053; NM_001320121; NM_001384361; |
| | NM_001320122; NM_006928 |
| SLAMF1 | NR_104400; XM_005245456; XM_017002130; NR_104401; NM_003037; |
| | XM_011509905; NM_001330754; NR_104399; XM_017002131 |
| SLC2A1 | NM_006516 |
| SLC2A2 | XM_024453720; XM_011513087; NM_000340; NM_001278659; NM_001278658 |
| SLC2A3 | NM_006931 |
| SLC2A4 | NM_001042 |
| SLC5A1 | NM_000343; XM_011530331; NM_001256314 |
| SLC5A2 | XM_006721072; NM_003041; NR_130783; XM_024450402 |
| SLC10A2 | NM_000452 |
| SLC16A1 | NM_003051; NM_001166496 |
| SLC25A1 | NM_001287387; NR_046298; NM_001256534; NM_005984 |
| SLC22A4 | XM_006714675; NM_003059; XM_011543589; XM_017009776 |
| SLC22A5 | XM_011543590; XM_017009778; XR_948291; NM_001308122; XR_001742215; |
| | XR_427718; XR_948290; XR_001742216; NM_003060 |
| SNAI2 | NM_003068 |
| SIGLEC1 | NM_001367089; NM_023068 |
| SNAI1 | NM_005985 |
| SNCB | NM_001318034; XM_006714916; NM_001001502; NM_001363140; |
| | XM_006714915; XM_011534640; NM_001318037; NM_003085; NM_001318035; |
| | XM_006714914; NM_001318036 |
| STAT4 | XM_011511705; NM_003151; XM_006712719; XM_017004784; NM_001243835 |
| XCL2 | NM_003175 |
| TALDO1 | NM_006755 |
| TAP1 | NM_000593; NM_001292022; |
| TAP2 | NM_001290043; NM_000544; NM_018833 |
| ZEB1 | NM_001174096; NM_001323650; NM_001323655; NM_001323660; |
| | NM_001323677; XM_006717498; NM_001174095; NM_001323638; |
| | NM_001323645; NM_001323647; NM_001323663; NM_001323666; |
| | NM_001323672; NM_030751; XM_011519643; NM_001323662; XM_017016597; |
| | NM_001128128; NM_001174094; NM_001323641; NM_001323644; |
| | NM_001323659; NM_001323674; NM_001323675; NM_001323676; |
| | NM_001323642; NM_001323648; NM_001323649; NM_001323652; |
| | NM_001323654; NM_001323671; NM_001323643; NM_001323658; |
| | NM_001323665; NM_001323651; NM_001323656; NM_001323661; |
| | NM_001323664; NM_001323673; NM_001323678; NM_001174093; |
| | NM_001323646; NM_001323653; NM_001323657 |
| TGFA | NM_001308159; NM_001308158; NM_001099691; NM_003236 |
| TGFB1 | NM_000660; XM_011527242 |
| TGFB2 | NM_003238; NR_138149; NR_138148; NM_001135599 |
| TGFB3 | NM_001329938; NM_003239; NM_001329939 |
| TGM2 | NM_004613; NM_001323316; XM_011529028; NM_001323318; NM_001323317; |
| | NM_198951 |
| TCHH | NM_007113 |
| TP73 | NM_001126242; NM_001204191; NM_001126240; NM_001204185; |
| | NM_001204187; NM_001204184; NM_001204186; NM_001204192; |
| | NM_001126241; NM_001204188; NM_001204189; NM_001204190; NM_005427 |
| TPI1 | NM_001159287; NM_000365; XR_002957378; NM_001258026 |
| TWIST1 | NR_149001; NM_000474 |
| TNFSF4 | XR_002957545; XM_017002229; XM_017002230; NM_003326; XR_001737393; |
| | XR_002957543; XR_001737394; NM_001297562; XM_017002228; |
| | XR_001737395; XM_011509964 |
| TNFRSF4 | XM_011542074; NM_003327; XM_017002232; XM_011542077; XM_011542075; |
| | XM_011542076; XM_017002231 |
| TXN | NM_001244938; NM_003329 |
| TXNRD1 | NM_001261446; NM_182742; NM_182743; NM_003330; NM_182729; |
| | NM_001093771; NM_001261445 |
| UCHL1 | NM_004181 |
| UGDH | NM_001184701; NM_001184700; NM_003359; XM_005262667 |
| VCAM1 | NM_080682; NM_001078; NM_001199834 |
| WNT10B | XM_011538724; XM_011538722; XM_017019919; XM_024449179; NM_003394 |
| ZAP70 | XM_017004868; XR_001738927; NM_001378594; NM_207519; XM_017004869; |
| | NM_001079; XR_001738926; XM_017004867; XM_017004870; XR_001738925 |
| LRP8 | NM_033300; NM_001018054; NM_004631; NM_017522 |
| CXCR4 | NM_001348059; NM_001348060; NM_001348056; NM_003467; NM_001008540 |
| ALDH5A1 | NM_170740; NM_001080; NM_001368954 |
| PDHX | NM_001166158; NM_001135024; XM_011520390; NM_003477 |
| MFAP5 | NM_001297709; NR_123733; NR_123734; NM_001297711; NM_003480; |
| | NM_001297710; NM_001297712 |
| SLC25A11 | NM_001165418; NM_003562; NM_001165417; XM_024450994 |
| SEMA7A | NM_001146030; NM_003612; NM_001146029 |
| CST7 | NM_003650 |
| PIR | NM_003662; NM_001018109 |
| TNFSF11 | XM_017020803; NM_033012; XM_011535280; XM_017020802; NM_003701 |
| RGS20 | NM_001286673; NM_001286675; NM_170587; NM_001286674; NM_003702; |
| | NR_104578; NR_104579 |
| PDE8B | XM_011543700; NM_001349751; NM_001376069; NM_001376072; NM_003719; |
| | NM_001349750; NM_001376063; XM_006714726; NM_001349753; |
| | NM_001376075; XM_011543699; NM_001029853; NM_001029854; |
| | NM_001376066; NM_001029851; NM_001349752; NM_001376073; |
| | XM_011543704; NM_001029852; NM_001349749; NM_001376067; |
| | NM_001349748; NM_001376062; NM_001376068; NM_001376070; |
| | NM_001376074; NM_001376064; NM_001376065; NM_001376071 |
| AKR1C3 | NM_003739; NM_016253; NM_001253909; NM_001253908 |
| PDE5A | XM_017008791; NM_033437; NM_001083; NM_033430 |
| ALDH4A1 | NM_001161504; NM_003748; NM_001319218; NM_170726 |
| TNFSF10 | NR_033994; NM_001190943; NM_003810; NM_001190942 |
| TNFSF9 | NM_003811 |
| TNFRSF18 | NM_148901; NM_004195; XM_017002722; NM_148902 |
| FBP2 | NM_003837 |
| SUCLG2 | XR_001740348; XM_017007420; NM_001177599; NM_003848; XR_001740350; |
| SUCLG1 | NM_003849 |
| SUCLA2 | NM_003850 |
| SLC5A6 | NM_021095; XR_001739023; XM_024453207; XR_002959356; XR_001739024; |
| | XM_006712129; XM_024453206; XR_001739025; XR_002959358; NR_028323; |
| | XM_017005216; XR_001739022; XM_006712128; XM_006712130; |
| | XM_011533146; XR_002959357 |
| P4HA2 | NM_004199; NM_001017974; NM_001365679; NM_001365681; NM_001142599; |
| | NM_001142598; NM_001365677; NM_001365678; NM_001365680 |
| PGLYRP1 | NM_005091 |
| SOCS3 | NM_001378933; NM_003955; NM_001378932 |
| SLC13A2 | NM_001145975; NM_001346683; NM_003984; NM_001145976; XM_006722165; |
| | XM_011525450; XM_011525453; XM_011525454; NM_001346684; |
| | XM_011525452; XM_011525451 |
| SLC16A3 | NM_001206952; NM_001206951; NM_004207; NM_001206950; XM_024451023; |
| | NM_001042423; NM_001042422 |
| KCNQ4 | NM_172163; XM_017002792; NM_004700 |
| SLC16A7 | XM_011538990; XM_011538992; NM_004731; NM_001270622; XM_017020225; |
| | XM_017020227; NR_073055; XM_011538989; NM_001270623; XM_024449276; |
| | XM_011538991; XM_011538993; NR_073056; XM_005269231; XM_011538995; |
| | XM_017020226; XM_017020224 |
| SLC33A1 | XM_017007464; XR_001740361; NM_001363883; XR_002959605; |
| | NM_001190992; XM_011513311; XR_001740362; NM_004733; XM_017007463 |
| CD83 | NM_001040280; NM_001251901; NM_004233 |
| CD163 | XR_002957389; XM_024449278; NM_203416; NM_001370145; NM_001370146; |
| | NM_004244; NR_163255 |
| NCR1 | XM_011527528; NM_004829; XM_011527530; NM_001145457; NM_001242357; |
| | XM_011527529; XR_001753801; NM_001242356; NM_001145458 |
| ADAMTS4 | XR_001737548; NM_001320336; NM_005099; XR_001737549 |
| GREB1 | XM_024453255; NM_014668; NM_033090; XM_024453254; XM_024453256; |
| | NM_148903; XM_005246196; XM_024453251; XR_922686; XM_024453250; |
| | XM_024453252; XM_011510418; XM_011510423; XM_011510422; |
| | XM_024453253; XM_005246192; XM_011510419; XR_001739081 |
| ZEB2 | NM_001171653; NM_014795; NR_033258 |
| KIAA0319 | XM_011515026; NM_001350407; NM_001350410; XM_017011551; |
| | NM_001168375; NM_001168377; XM_017011546; XM_017011547; |
| | XR_001743779; NM_001168376; NM_001350403; XM_017011542; |
| | XM_017011550; NM_001350404; XM_017011544; NM_001168374; |
| | XM_017011541; XR_001743780; NM_001252328; NM_001350405; |
| | NM_001350406; NM_001350408; NM_001350409; NM_014809 |
| SV2B | XM_017022762; NM_001323038; NM_001323040; NM_001323032; NM_014848; |
| | XM_005254998; NM_001323036; NM_001167580; NM_001323033; |
| | XM_017022761; NM_001323031; NM_001323034; NM_001323037; |
| | NM_001323039 |
| HCN4 | XM_011521148; NM_005477 |
| CCL26 | NM_006072; NM_001371936; NM_001371938 |
| TUBB4A | NM_001289129; NM_001289131; NM_006087; NM_001289123; NM_001289127; |
| | NM_001289130 |
| FST | XM_005248403; XM_011543099; XM_005248400; XM_017008955; NM_013409; |
| | XM_005248401; XM_005248402; XM_017008954; XM_024454326; NM_006350 |
| HOXB13 | NM_006361 |
| SEMA4B | NM_001324030; NM_001324032; NM_001324034; NM_001324031; NM_020210; |
| | NM_001393916; NM_001324029; NR_172049; NM_198925 |
| TRIM16 | NM_001348124; NM_001348120; NM_001348121; NM_001348126; |
| | NM_001348122; NM_006470; NM_001348119; NM_001348125 |
| CD226 | XM_017025527; NM_006566; NM_001303619; XM_017025525; XM_006722374; |
| | XM_017025526; XM_005266642; NM_001303618; XM_005266643 |
| PDE10A | XM_011535388; XR_001743121; NM_001385079; XM_017010195; |
| | XM_011535393; XM_011535387; XM_017010194; XM_017010196; |
| | XM_024446312; NM_001130690; NM_006661; NR_045597; XM_006715321; |
| | XM_017010197; XM_024446311 |
| ME3 | XM_017017138; XR_001747744; XR_949761; XR_949766; XR_001747738; |
| | XR_001747740; NR_147829; NR_172889; XR_001747739; NM_001351934; |
| | XM_017017137; XR_001747743; NR_147831; XR_001747746; NM_001161586; |
| | NR_147830; XR_001747737; XR_001747741; XR_001747745; XR_949760; |
| | NM_001014811; NM_001395868; NR_147828; XR_001747742; XR_001747747; |
| | XR_001747736; XR_001747749; NM_006680; NR_172888 |
| SLC27A5 | NM_012254; XM_011526364; XM_017026214; NM_001321196 |
| SLC27A4 | XM_017014222; NM_005094; XM_024447391 |
| SLC27A3 | NR_145826; NM_024330; NM_001317929 |
| SLC27A2 | NM_001159629; NM_003645 |
| ADAMTS5 | XM_024452053; XM_024452054; NM_007038 |
| CD160 | NM_007053; XM_005272929; XM_011509104; NR_103845 |
| SLC2A6 | XR_001746173; XM_011518189; XM_017014238; NM_001145099; |
| | XM_017014237; XR_001746175; XR_001746172; XM_017014236; XR_001746174; |
| | NM_017585 |
| IKZF2 | XM_011510804; XM_011510815; NM_001371277; XM_011510803; |
| | XM_011510808; XM_011510816; NM_001371275; XM_005246386; |
| | XM_011510807; XM_011510810; XM_011510811; NM_001371276; |
| | XM_005246385; XM_011510809; NM_001079526; NM_001371274; |
| | NM_001387220; NM_016260; XM_005246384; XM_011510817; XM_011510818; |
| | XM_017003591; XM_011510805; XM_011510812; XM_011510819; |
| | XM_017003592; XM_011510802 |
| MMRN1 | XM_005262856; NM_001371403; NM_007351 |
| PTGR1 | XM_017014485; XR_001746250; NM_012212; XM_011518394; XM_011518395; |
| | XR_929738; NM_001146108; NM_001146109 |
| MLYCD | NM_012213 |
| TNFRSF13B | NM_012452 |
| SLC16A8 | NM_001394131; XM_017028685; NM_013356 |
| NTSR2 | NM_012344; XM_005246156; XM_006711877; XM_006711876; XM_017003738 |
| SLC7A11 | XR_001741190; XM_011531802; XR_001741191; NM_014331 |
| CFAP61 | NM_001167816; NM_015585 |
| STAP1 | NM_001317769; NM_012108; XM_017008018 |
| CABYR | XM_024451161; NM_001308231; NM_153770; XM_024451163; NM_153768; |
| | XM_005258247; XM_024451162; NM_138644; XM_024451160; XM_024451159; |
| | NM_012189; NM_138643; NM_153769 |
| PLA2G2D | NM_001271814; NM_012400 |
| GAPDHS | NM_014364 |
| PDE7B | NM_018945 |
| NMRK2 | NM_001289117; NM_001375468; NM_001375469; NM_170678; NM_014446; |
| | NM_001375467; XM_006722725; NR_110316 |
| CD274 | NM_001314029; NM_001267706; NR_052005; NM_014143 |
| OSGIN1 | NM_013370; NM_182980; NM_182981 |
| SLC2A8 | XM_017014653; XR_002956777; XM_011518603; NM_014580; NR_073416; |
| | NM_001271712; XM_011518604; XM_024447526; XM_011518602; |
| | NM_001271711; XM_006717084; XR_929783 |
| TBX21 | NM_013351 |
| IL22 | NM_020525 |
| ARHGEF4 | NM_001375900; NM_001375902; NM_001375901; NM_001375904; |
| | NM_001367493; NM_001375903; NM_015320; NM_001395416; NM_032995 |
| TRAT1 | NM_016388; NM_001317747 |
| PDE11A | NM_001077196; NM_001077197; NM_016953; NM_001077358 |
| FOXP3 | XM_006724533; NM_001114377; NM_014009; XM_017029567 |
| SLC45A2 | NM_001297417; NM_016180; NM_001012509 |
| MZB1 | NM_016459 |
| KLRF1 | XM_017019415; NM_001291822; XR_931301; NM_001366534; NR_120305; |
| | NM_001291823; NM_016523; NR_159359; NR_159360; NR_159361 |
| IL23A | XM_011538477; NM_016584 |
| ACSL5 | NM_001387037; NM_016234; NM_203379; NM_203380 |
| CD244 | NM_001166663; XM_011509622; XR_001737229; NM_016382; NM_001166664; |
| | XM_011509623; XM_011509621 |
| NECAB2 | NM_001329748; XM_017023317; NM_001329749; NM_019065 |
| CROT | NM_021151; XM_017012370; NM_001143935; XM_011516337; NM_001243745 |
| LAX1 | NM_001136190; NM_001282878; NM_017773; XM_006711397 |
| PGM2 | XM_011513711; NM_018290 |
| UEVLD | NM_001261384; NM_001261386; NM_001297771; NM_001040697; NM_018314; |
| | NM_001040698; NM_001261385; NM_001261382; NM_001261383 |
| ADCY10 | XM_006711449; XM_011509760; XM_011509763; XM_005245330; |
| | XM_011509766; NM_001167749; NM_018417; XR_921889; NM_001297772; |
| | XM_011509762; XM_017001778 |
| ALLC | XM_017004495; XM_017004498; NM_018436; XM_011510369; XM_017004496; |
| | XM_011510370; XM_017004497; NM_199232 |
| ACSS2 | NM_018677; XM_011528908; XM_011528912; NM_001242393; NM_139274; |
| | NM_001076552; XM_005260455; XM_005260456; XM_011528907; |
| | XM_011528911; XM_011528906; XM_011528905; XM_006723826; |
| | XM_011528909 |
| CCL28 | NM_001301874; NM_001301873; NM_019846; XR_925633; NM_148672; |
| | XR_427660; NM_001301875; XR_241706 |
| SLC2A9 | XM_011513857; XM_011513858; XM_011513859; XM_011513866; |
| | XM_011513860; XM_011513865; XM_017008460; NM_020041; XM_011513861; |
| | XM_011513862; XM_024454151; NM_001001290; XM_011513864; |
| | XM_011513867; XM_011513868; XM_017008457; XM_024454153; |
| | XM_011513856; XR_925341; XM_006713968; XM_006713969; XR_001741291; |
| | XM_017008458; XM_024454152; XM_017008459; XM_024454150; |
| | XR_001741290 |
| PANX2 | NM_001160300; NR_027691; NM_052839 |
| AKR1B10 | XR_927491; XM_011516416; XM_011516417; NM_020299 |
| CD248 | NM_020404 |
| CD177 | XM_017027021; XM_017027022; NM_020406 |
| CASKIN1 | XM_024450361; NM_020764 |
| KIAA1549 | NM_001164665; XM_011516442; NM_020910 |
| BRINP2 | XM_005245379; NM_021165; XM_011509826; XM_024448722 |
| G6PC2 | XM_011511565; NM_021176; NM_001081686; XM_011511564 |
| SLAMF7 | XM_011509828; XM_011509829; NM_001282589; NM_001282590; |
| | NM_001282596; XM_024448757; NM_001282591; NM_001282593; |
| | NM_001282588; NM_001282595; NM_001282592; NM_001282594; NM_021181 |
| CATSPERG | NM_021185; NM_001330496 |
| CYP4F11 | NM_001128932; NM_021187 |
| IL21 | NM_021803; NM_001207006 |
| ABHD4 | XR_001750499; NR_171624; NM_022060; NR_171625; NM_001392009; |
| | NR_171626 |
| IKZF4 | XM_017019810; XM_017019813; XM_017019814; XM_017019809; |
| | NM_001351089; NM_022465; XM_017019807; XM_017019815; XM_017019816; |
| | XM_024449130; NM_001351090; NM_001351091; XM_011538664; |
| | XM_011538669; XM_017019806; XM_017019808; XM_017019811; |
| | XM_005269086; XM_005269089; XM_024449128; XM_024449129; |
| | XM_024449131; NM_001351092; XM_017019812 |
| CPEB1 | NM_001288819; NM_001365242; NM_001365243; NM_001365244; |
| | NM_001365245; NM_001387068; NM_001387076; NM_001365248; |
| | NM_001079534; NM_001365250; NM_001387065; NM_001387075; |
| | NM_001079535; NM_001288820; NM_001365249; NM_001387061; |
| | NM_001387066; NM_001387070; NM_001387062; NM_001387071; |
| | NM_001387078; NM_001365246; NM_001365247; NM_001387069; |
| | NM_001387077; NM_001079533; NM_001365240; NM_001365241; |
| | NM_001387072; NM_001387074; NM_001387063; NM_001387064; |
| | NM_001387067; NM_001387073; NM_030594 |
| CCDC136 | XM_011516484; XM_024446872; XM_024446875; NM_001363423; NM_022742; |
| | XM_024446873; NM_001367763; NM_001367764; NM_001367765; |
| | NM_001367766; XM_011516489; XM_011516483; XM_011516486; |
| | NM_001363424; XM_011516485; XM_017012532; XM_017012533; |
| | NM_001201372; XM_011516487; XM_011516488; XM_011516490; |
| | XM_024446874; NM_001367761; NM_001367762 |
| SLC13A3 | NM_001011554; NM_001193339; NM_022829; NM_001193342; NM_001193340 |
| NDRG4 | NM_001242833; NM_001378335; NM_001242836; NM_001378334; |
| | NM_001378338; NM_001378341; NR_040072; XM_006721253; XM_011523290; |
| | XM_024450398; XM_024450400; NM_001378343; NM_001378347; NM_020465; |
| | XM_006721256; XM_017023582; NM_001130487; NM_001242834; |
| | NM_001242835; NM_001378332; NM_001378333; NM_001378339; |
| | NM_001378345; NM_001378346; XM_011523294; NM_022910; XM_011523293; |
| | NM_001378342; NM_001378344; XM_017023583; XM_011523291; |
| | NM_001363869; NM_001378336; NM_001378337; NM_001378340 |
| CYP4F12 | XM_006722850; XM_011528204; XM_011528208; NM_023944; XM_011528207; |
| | XM_011528202; XM_017027172; XM_011528203; XM_011528205; NR_117085 |
| SLC2A11 | NM_001282864; NM_001024939; NM_030807; NM_001024938; NR_104248; |
| | NR_104247 |
| BIRC7 | NM_022161; NM_139317 |
| MMRN2 | XM_005270153; NM_024756 |
| SNX22 | NM_024798; XM_005254677; XM_017022581; NR_073534 |
| HKDC1 | XR_001747209; NM_025130; XM_011540195 |
| SCUBE1 | NR_147916; NM_173050; NM_152514 |
| PDCD1LG2 | XM_005251600; NM_025239 |
| SLC2A10 | XR_936641; XM_011529062; XM_011529060; XM_011529065; NM_030777; |
| | XM_011529064; XM_011529061; XM_011529063; XM_017028087 |
| SPACA1 | NM_030960; XM_017011335; XM_011536160; XR_241854 |
| FAHD1 | NM_001142398; NM_001018104; NM_031208 |
| FCRL5 | XM_011510032; XM_011510030; XM_011510031; XM_011510033; NM_031281; |
| | NM_001195388 |
| INHBE | NM_031479 |
| FGFBP2 | NM_031950 |
| NLRC5 | XM_005256196; XM_011523373; XR_001751997; NM_001330552; |
| | NM_001384961; NM_001384969; NM_001384972; NM_001384973; NR_169518; |
| | XM_005256201; XR_001751999; XR_001752000; NM_001384951; |
| | NM_001384959; NM_032206; NR_169513; XM_006721300; XM_017023770; |
| | NM_001384950; NM_001384958; NM_001384964; NR_169520; XM_017023771; |
| | XR_001751998; NM_001384954; NM_001384966; NR_169514; NR_169517; |
| | NM_001384967; NM_001384971; XM_006721298; XR_429734; NM_001384965; |
| | NM_001384970; NR_169512; NR_169519; XM_005256194; XM_011523376; |
| | NM_001384953; NM_001384956; NM_001384957; NM_001384962; |
| | NM_001384963; NR_169515; NR_169516; NM_001384952; NM_001384955; |
| | NM_001384960; NM_001384968 |
| SYT3 | NM_001160328; NM_001160329; NM_032298 |
| ACSS1 | XM_011529388; NM_001252676; NM_001252677; XR_937170; XM_006723659; |
| | NM_001252675; NM_032501 |
| TRIM63 | NM_032588 |
| CBX2 | XM_011525383; NM_032647; NM_005189; XM_011525382 |
| HAVCR2 | NM_032782 |
| AIFM2 | NM_001198696; NM_032797 |
| HSH2D | NM_001369808; NM_001382417; NR_111904; NM_001352266; NR_111903; |
| | NR_163156; NM_001352265; NR_163153; NR_163155; NR_163150; |
| | NM_001369809; NM_032855; NR_163151; NR_163154; NR_163152 |
| EPT1 | NM_033505; NR_137633 |
| IGFN1 | XM_006711618; XM_011510120; NM_001367841; XM_011510121; |
| | XM_017002787; NM_001164586; XM_005245580; NM_178275; XR_921994 |
| LDHAL6B | NM_033195 |
| G6PC3 | NM_001319945; NM_001384165; NM_001384166; NM_001384167; |
| | NM_001384168; XM_011525474; NM_138387 |
| PKD1L2 | NM_001278425; NM_052892; NM_001278423; NM_001076780; NR_126532; |
| | NM_182740 |
| SLC5A11 | XM_011545725; NM_001258412; NM_001352236; NM_001352242; NR_147939; |
| | NM_001258413; NM_001352250; NM_001394079; NM_001394080; NR_147934; |
| | NR_147938; NM_001352248; NM_001394077; NR_147935; NM_001352237; |
| | NM_001394078; XM_024450145; NM_001258411; NM_001352235; |
| | NM_001352238; NR_172075; NR_172077; NM_001352245; NM_001352259; |
| | NM_001394076; NR_147933; NR_147936; XM_011545728; XM_017022903; |
| | XM_024450146; NM_001258414; NM_001352246; NM_001352249; |
| | NM_001352247; NR_147940; NR_172076 |
| TNFRSF13C | NM_052945 |
| SH2D1B | NM_053282 |
| IP6K3 | XM_005248843; XM_024446323; NM_001142883; XM_024446326; NM_054111; |
| | XM_024446327; XM_011514295; XM_024446324; XM_024446325; |
| | XM_005248842 |
| TWIST2 | NM_057179; NM_001271893 |
| SLC51B | XM_005254159; NM_178859 |
| JSRP1 | XM_017026286; XM_017026285; NM_144616; XM_017026287; |
| PLK5 | NM_001243079; NR_026557; NM_001011716 |
| C1orf131 | NM_001300830; NM_152379 |
| MDH1B | XR_922861; XR_001738631; NM_001282940; NR_104261; NR_138467; |
| | XM_005246316; NM_001330226; NR_138468; NM_206892; NM_001039845; |
| | NM_001330223; NM_001330224; NM_001330225 |
| TRIM71 | NM_001039111 |
| SRXN1 | NM_080725 |
| SLC2A14 | XM_011520562; XM_011520563; NM_001286235; XM_017018846; |
| | XM_005253315; XM_017018841; XM_017018847; NM_001286234; |
| | NM_001286237; XM_011520565; NM_153449; XM_005253317; XM_011520564; |
| | XM_017018844; XM_017018845; XM_024448848; XM_024448849; |
| | NM_001286233; NM_001286236 |
| NRG4 | XM_017021938; XM_017021941; XM_017021944; XM_024449847; |
| | XM_017021945; XM_017021947; XM_017021940; XM_017021943; |
| | XM_017021939; XM_017021942; NM_138573; XM_017021937; XM_017021946; |
| | XM_017021948; XM_024449848 |
| TRIM16L | NM_001353219; NM_001353224; NR_172634; NR_172635; NR_172639; |
| | NR_172641; NM_001353221; NR_172638; NM_001353223; NR_172640; |
| | NM_001353220; NM_001353225; NR_172633; NM_001037330; NM_001353222; |
| | NM_001353226; NR_172636; NR_172637 |
| UBE2U | XM_017000381; XM_017000384; NM_001366233; XM_006710379; |
| | XR_002959500; NM_001366234; XM_017000380; XM_017000382; |
| | XM_017000385; XM_017000379; NM_001366235; NM_152489; NR_158769; |
| | NM_001366232; NR_158768; XM_011540764; XM_017000383 |
| SLC2A12 | XM_006715349; XM_017010311; NM_145176 |
| SLC2A7 | XM_011540825; NM_207420; XM_011540824 |
| ESCO2 | NM_001017420; XR_949378; XM_011544422; XM_011544421 |
| SLC5A12 | XM_006718156; XR_930845; XM_011519920; XM_011519921; NM_178498; |
| | XM_017017244; XM_006718155 |
| LDHAL6A | XM_005252805; XM_011519923; NM_001144071; XM_011519924; |
| | XM_011519925; XM_011519922; NM_144972 |
| SLC5A8 | XR_944503; XM_017018910; NM_145913 |
| CLEC14A | NM_175060 |
| C1QL2 | NM_182528 |
| CLYBL | NM_001393357; NM_001393355; NM_001393360; NM_001393361; |
| | NM_001393358; NM_001393359; NM_001393356; NM_206808; NM_001393362; |
| | NR_104592 |
| ANTXRL | XM_011539433; XM_011539440; XM_006717684; XM_011539434; |
| | XM_011539443; XM_017015818; XM_011539438; XM_011539430; XR_945614; |
| | XM_011539431; XM_017015819; XR_945612; NM_001278688; XM_011539437; |
| | XM_011539439; XM_011539441; NM_001354208; XM_011539442; XR_945615; |
| | NR_003601 |
| ADCY4 | NM_001198592; NM_001198568; NM_139247 |
| LDHD | XM_024450179; XM_024450175; XM_024450176; XM_024450177; |
| | XM_024450178; NM_153486; XM_024450174; NM_194436 |
| SLC5A9 | XR_946573; XM_011540927; XM_011540924; XM_011540925; XM_011540926; |
| | XM_011540928; XM_017000558; NM_001011547; XM_011540929; |
| | NM_001135181 |
| PLD5 | NM_152666; NM_001195811; XM_011544115; XM_011544116; XM_011544122; |
| | XM_017000569; NM_001320272; NM_001195812; XM_017000568; |
| | NM_001372062; XM_011544119; XM_011544120; XM_011544121; |
| | XM_024453867; XM_017000567; XM_017000570 |
| LGI3 | NM_139278 |
| FAM124A | XM_011534978; NM_001330522; XM_017020419; NM_001242312; NM_145019 |
| ZNF683 | XM_011541198; XM_005245830; XM_017000956; NM_001114759; NM_173574; |
| | XM_017000959; XM_005245832; XM_005245828; XM_006710555; |
| | XM_017000954; XM_017000955; XM_017000957; XM_017000958; |
| | NM_001307925 |
| NCR3 | NM_001145467; XM_011514459; XM_006715049; NM_001145466; NM_147130 |
| IL4I1 | NM_001385639; NM_172374; NM_152899; NR_047577; NM_001258018; |
| | NM_001258017 |
| JAKMIP3 | XM_005252676; XM_017016098; NM_001323087; NM_001392040; |
| | NM_001392052; NM_001392053; NM_001392064; NM_001392066; |
| | XM_011539676; XM_017016099; NM_001105521; NM_001323086; |
| | NM_001392048; NM_001392054; NM_001392059; NM_001392061; |
| | NM_001392062; NM_001392063; NM_001392039; NM_001392043; |
| | NM_001392046; NM_001392065; NM_001392042; NM_001392044; |
| | NM_001392045; NM_001323088; NM_001323089; NM_001392056; |
| | NM_001392057; NM_001392058; NM_001392067; NM_001323090; |
| | NM_001392041; NM_001392049; NM_001392055; XM_005252674; |
| | XM_017016095; NM_001104947; XM_011539682; NM_194303; NM_001392047; |
| | NM_001392050; NM_001392051; NM_001392060 |
| SLC13A5 | NM_177550; XM_011523795; NM_001284510; NM_001143838; NM_001284509; |
| LUZP2 | NM_001252008; XM_017017648; XR_930864; NM_001252010; XM_011520056; |
| | XM_017017649; NM_001009909 |
| ABCB5 | NM_001163941; NM_001163993; NM_178559; NM_001163942; XM_011515367 |
| IGFBPL1 | XM_017014699; NM_001007563 |
| SLC27A1 | XM_011528003; NM_198580; XM_011528000; XR_001753680; XM_017026781; |
| | XM_011528001; XM_011528002; XR_001753681 |
| CCL4 | NR_111969; NM_207007 |
| C1QL3 | NM_001010908 |
| TRIM67 | XM_011544192; NM_001300889; XM_017001323; NM_001004342 |
| AKR1B15 | NM_001367821; NM_001367820; NR_160301; NR_160302; XM_017012224; |
| | NM_001080538; NM_001367822 |
| CCR2 | NM_001123041; XM_011534069; NM_001123396 |
| PLIN4 | XM_017027192; NM_001393889; XM_011528233; NM_001393888; |
| | XM_017027193; NM_001080400; NM_001393890; XM_017027194; |
| | NM_001367868; NM_001393891 |
| ERVV-2 | NM_001191055 |
| KLRK1 | NM_001199805 |
| AURKB | NM_001313950; NM_001313953; XM_017025311; XM_017025307; |
| | XM_017025308; XM_017025309; NM_001313952; NM_004217; NM_001313954; |
| | NR_132730; NR_132731; XM_017025310; NM_001284526; XM_011524072; |
| | NM_001256834; NM_001313951; NM_001313955 |
| BLK | NM_001330465; XM_011543829; XM_011543824; XM_011543827; |
| | XM_011543828; NM_001715; XM_011543825 |
| BTLA | NM_001085357; NM_181780; XM_011512447; XM_017005748 |
| CIITA | XR_932842; NM_001379332; XM_006720880; XM_011522491; XR_932846; |
| | NM_001379334; XM_011522487; XR_932847; NM_001379333; XM_011522486; |
| | XM_011522494; XM_024450280; XR_932841; NM_000246; NM_001286402; |
| | XM_011522489; XM_024450281; XR_001751904; NM_001286403; |
| | NM_001379331; XM_011522485; NR_104444; XM_011522484; XM_011522490; |
| | NM_001379330 |
| CTSG | NM_001911; XM_011536499 |
| CYBB | NM_000397 |
| ELANE | XM_011527776; XM_011527775; NM_001972 |
| EOMES | NM_001278182; XM_005265510; NM_005442; NM_001278183 |
| FAP | XM_011510797; NM_004460; XM_011510796; XR_001738668; XM_017003585; |
| | XR_922891; NM_001291807 |
| FASLG | NM_001302746; NM_000639 |
| GNLY | XM_005264085; NM_001302758; XM_005264087; NM_006433; XM_005264084; |
| | NM_012483 |
| GZMA | NM_006144 |
| GZMH | NM_001270781; XM_011536683; NM_033423; NM_001270780 |
| GZMK | NM_002104 |
| HLA-A | XM_041680767; XR_005976896; NM_001242758; XM_041680768; NM_002116 |
| HLA-B | NM_005514 |
| HLA-DMA | NM_002118 |
| HLA-DMB | NM_002118.5 |
| HLA-DRA | NM_019111 |
| ICOS | NM_012092 |
| ICOSLG | NM_001395918; NM_001283050; XM_011529514; NM_001283051; |
| | NM_001283052; NM_015259; XM_024452060; NM_001365759; XM_011529516 |
| IFNG | NM_000619 |
| ITK | NM_005546; XM_017009443 |
| KDR | NM_002253 |
| LDHB | NM_001315537; NM_002300; XM_006719074; NM_001174097 |
| LOX | NM_001317073; NM_002317; NM_001178102 |
| LUM | NM_002345 |
| MPO | NM_000250 |
| PDGFC | XM_011532124; XM_017008456; XM_017008455; NM_016205; NR_036641 |
| PDGFRA | NM_001347828; NM_001347829; XM_005265743; XM_017008281; |
| | NM_001347827; NM_001347830; NM_006206; XM_006714041 |
| TAPBP | XM_017011227; XM_011514828; NM_003190; NM_172208; NM_172209 |
| TEK | NM_001375475; NM_000459; NM_001290077; NM_001290078; NM_001375476 |
| TIGIT | XR_002959502; NM_173799; XM_024453388 |
| TNC | XM_005251975; XM_006717096; XM_011518628; XM_017014681; |
| | XM_011518626; XM_005251973; XM_006717098; XM_005251972; |
| | XM_006717097; XM_011518629; XM_017014680; XM_011518625; |
| | XM_017014679; XM_005251974; XM_006717101; XM_017014678; |
| | XM_024447530; NM_002160 |
| TNF | NM_000594 |
| VEGFA | NM_001171625; NM_003376; NM_001033756; NM_001171624; NM_001171626; |
| | NM_001171630; NM_001025366; NM_001317010; NM_001025368; |
| | NM_001025370; NM_001171623; NM_001171622; NM_001171628; |
| | NM_001171629; NM_001204385; NM_001025367; NM_001025369; |
| | NM_001171627; NM_001204384; NM_001287044 |
| VEGFB | NM_003377; NM_001243733 |
| VEGFC | NM_005429 |
| VSIR | NM_022153 |
| VTN | NM_000638 |
| VWF | NM_000552 |
| AURKA | NM_001323304; NM_001323303; NM_198435; NM_198437; XM_024451974; |
| | NM_198433; NM_198434; NM_198436; XM_017028034; XM_017028035; |
| | NM_001323305; NM_003600 |
| HLA-C | NM_002117; NM_001243042 |
| MIF | NM_002415 |
| PDGFRB | NM_001355016; NM_002609; NM_001355017; NR_149150 |
| PGF | NM_002632; NM_001293643; NM_001207012 |
| TRAC | X02592.1 |
| FTHL3 | NR_002201.1 |
| GUCY1B2 | NR_003923.2 |
| SLC5A3 | NM_006933.7 |
| TRBC1 | ENST00000633705.1 |
| TRBC2 | ENST00000466254.1 |
| GZMB | NR_144343.2; NM_004131.6; NM_001346011.2 |

TABLE 4
Representative NCBI Accession Numbers for genes listed in Table 2

| gene name | mRNA Accession |
|---|---|
| CASQ1 | NM_001231.5 |
| TNNI1 | NM_003281.4 |
| MB | NM_001382813.1; NM_203377.1; NM_001382809.1; NM_001382812.1; |
| | NM_001362846.2; NM_001382810.1; NM_001382811.1; NM_203378.1; NM_005368.3 |
| MYLPF | NM_001324459.2; NM_001324458.2; NM_013292.5 |
| MYH7 | XM_017021340.1; NM_000257.4 |
| CKM | NM_001824.5 |
| MYL2 | NM_000432.4 |
| MYL1 | NM_079420.3; NM_079422.3 |
| CSRP3 | NM_001369404.1; NM_003476.5 |
| ACTA1 | NM_001100.4 |
| MYOZ1 | NM_021245.4 |
| TNNT3 | XM_017018206.1; XM_024448669.1; XM_017018207.1; XM_017018209.1; |
| | XM_017018211.1; XM_006718294.3; XM_017018205.1; XM_006718300.3; |
| | XM_017018216.1; XM_017018217.1; XM_017018218.1; XM_017018219.1; |
| | XM_006718299.2; NM_001297646.2; NM_001367846.1; NM_001363561.2; |
| | NM_001042781.3; NM_001367848.1; NM_006757.4; NM_001042780.3; |
| | NM_001042782.3; NM_001367845.1; NM_001367850.1; NM_001367851.1; |
| | XM_011520343.2; NM_001367847.1; NM_001367843.1; NM_001367842.1; |
| | NM_001367844.1; NM_001367849.1; NM_001367852.1; XM_017018208.1; |
| | XM_017018214.1; XM_017018210.1; XM_024448671.1; XM_024448670.1; |
| | XM_017018212.1; XM_017018213.1; XM_017018215.1; XM_024448672.1; |
| | XM_006718288.3; |
| TNNC2 | XM_011529031.2; NM_003279.3 |
| TNNC1 | NM_003280.3 |

1. A method for determining a renal cancer (RC) tumor microenvironment (TME) type for a subject having, suspected of having, or at risk of having renal cancer, the method comprising:
using at least one computer hardware processor to perform:
(a) obtaining RNA expression data for the subject, the RNA expression data indicating RNA expression levels for at least some genes in each group of at least some of a plurality of gene groups listed in Table 1;
(b) generating an RC TME signature for the subject using the RNA expression data, the RC TME signature comprising gene group scores for respective gene groups in the at least some of the plurality of gene groups, the generating comprising:
determining the gene group scores using the RNA expression levels; and
(c) identifying, using the RC TME signature and from among a plurality of RC TME types, an RC TME type for the subject.

2-14. (canceled)

15. The method of claim 1, wherein determining the gene group scores comprises:
determining a respective gene group score for each of at least two of the following gene groups, using, for a particular gene group, RNA expression levels for at least three genes in the particular gene group to determine the gene group score for the particular group, the gene groups including:
(a) Effector cells group: PRF1, GZMB, TBX21, CD8B, ZAP70, IFNG, GZMK, EOMES, FASLG, CD8A, GZMA, GNLY;
(b) NK cells group: GZMB, NKG7, CD160, GZMH, CD244, EOMES, KLRK1, NCR1, GNLY, KLRF1, FGFBP2, SH2D1B, KIR2DL4, IFNG, NCR3, KLRC2, CD226;
(c) T cells group: TRAC, TRBC2, TBX21, CD3E, CD3D, ITK, TRBC1, CD3G, CD28, TRAT1, CD5;
(d) B cells group: CR2, MS4A1, CD79A, FCRL5, STAP1, TNFRSF17, TNFRSF13B, CD19, BLK, CD79B, TNFRSF13C, CD22, PAX5;
(e) Antitumor cytokines group: IFNA2, CCL3, TNF, TNFSF10, IL21, IFNB1;
(f) Checkpoint inhibition group: CTLA4, HAVCR2, CD274, LAG3, BTLA, VSIR, PDCD1LG2, TIGIT, PDCD1;
(g) Treg group: TNFRSF18, IKZF2, IL10, IKZF4, CTLA4, FOXP3, CCR8;
(h) Neutrophil signature group: FCGR3B, CD177, CTSG, PGLYRP1, FFAR2, CXCR2, PRTN3, ELANE, MPO, CXCR1;
(i) Granulocyte traffic group: CXCL8, CCR3, CXCR2, CXCL2, CCL11, KITLG, CXCL1, CXCL5, CXCR1;
(j) MDSC group: ARG1, IL4I1, IL10, CYBB, IL6, PTGS2, IDO1;
(k) Macrophages group: MRC1, CD163, MSR1, SIGLEC1, IL4I1, CD68, IL10, CSF1R;
(l) Cancer-associated fibroblasts (CAF) group: PDGFRB, COL6A3, FBLN1, CXCL12, COL6A2, COL6A1, LUM, CD248, COL5A1, MMP2, COL1A1, MFAP5, PDGFRA, LRP1, FGF2, MMP3, FAP, COL1A2, ACTA2;
(m) Matrix group: COL11A1, LAMB3, FN1, COL1A1, COL4A1, ELN, LGALS9, LGALS7, LAMC2, TNC, LAMA3, COL3A1, COL5A1, VTN, COL1A2;
(n) Angiogenesis group: PGF, CXCL8, FLT1, ANGPT1, ANGPT2, VEGFC, VEGFB, CXCR2, VEGFA, VWF, CDH5, CXCL5, PDGFC, KDR, TEK;
(o) Endothelium group: NOS3, MMRN1, FLT1, CLEC14A, MMRN2, VCAM1, ENG, VWF, CDH5, KDR;
(p) Proliferation rate group: AURKA, MCM2, CCNB1, MYBL2, MCM6, CDK2, E2F1, CCNE1, ESCO2, CCND1, AURKB, BUB1, MKI67, PLK1, CETN3;
(q) EMT signature group: SNAI2, TWIST1, ZEB2, SNAI1, ZEB1, TWIST2, CDH2;
(r) Citric Acid Cycle group: ACLY, FAH, PC, MDH1B, SLC16A7, IREB2, PCK1, MDH1, SLC33A1, ALDH1B1, IDH3B, DLST, PDHB, MDH2, ACO1, IDH1, SLC5A6, HICDH, SLC16A8, GOT1, ME3, ME1, CS, OGDH, SDHA, ALDH5A1, CLYBL, SDHD, IDH3A, SLC25A1, ACSS2, SDHC, ACSS1, SUCLA2, SLC13A5, PDHX, SDHB, ALDH4A1, PCK2, DLD, ACO2, PDHA1, SLC13A2, FAHD1, IDH2, GOT2, ME2, ADSL, SUCLG2, SLC13A3, SUCLG1, SLC25A10, FH, IDH3G, SLC16A1, SLC25A11, PDHA2, DLAT;
(s) Glycolysis and Gluconeogenesis group: SLC2A9, PFKL, GCK, PFKFB4, SLC16A7, PCK1, PGAM2, GAPDH, BPGM, G6PC2, FBP2, LDHD, SLC2A3, GPI, ENO1, SLC25A11, PFKFB3, PFKM, LDHAL6B, SLC2A2, G6PC3, SLC2A6, GAPDHS, SLC2A11, PCK2, PFKP, PGK1, ALDOC, SLC2A10, ACYP2, SLC2A4, PKLR, HKDC1, PGK2, SLC2A8, PGAM1, SLC5A1, SLC5A12, SLC16A1, ALDOB, HK3, HK1, SLC5A9, GPD2, PFKFB1, SLC2A7, SLC5A11, SLC5A3, ACYP1, SLC16A8, PFKFB2, ALDOA, SLC5A2, HK2, ENO3, SLC2A12, FBP1, LDHA, LDHB, LDHC, G6PC, SLC2A14, SLC5A8, TPI1, SLC16A3, PKM2, ENO2, PGM1, UEVLD, LDHAL6A, SLC2A1, PGM2; and
(t) Fatty Acid Metabolism group: MLYCD, ALDH3A2, SLC27A5, SLC27A3, LIPC, SLC27A2, ACSL4, ACSL1, PCCB, SLC25A20, AADAC, SLC22A4, SLC22A5, ECH1, PCCA, SLC27A1, SLC27A4, CROT, ACSL5, ACSL3, CYP4F12.

16. (canceled)

17. The method of claim 15, wherein determining the gene group scores further comprises:
determining a respective gene group score for each of at least two of the following gene groups, using, for a particular gene group, RNA expression levels for at least three genes in the particular gene group to determine the gene group score for the particular group, the gene groups including:
(a) ECM associated group: ADAM8, ADAMTS4, C1QL3, CST7, CTSW, CXCL8, FASLG, LTB, MUC1, OSM, P4HA2, SCUBE1, SEMA4B, SEMA7A, SERPINE1, TCHH, TGFA, TGM2, TNFSF11, TNFSF9, WNT10B;
(b) TLS kidney group: ZNF683, POU2AF1, LAX1, CD79A, CXCL9, XCL2, JCHAIN, SLAMF7, CD38, SLAMF1, TNFRSF17, IRF4, HSH2D, PLA2G2D, MZB1;
(c) NRF2 signature group: TRIM16L, UGDH, KIAA1549, PANX2, FECH, LRP8, AKR1C2, FTH1, AKR1C3, CBR1, PFN2, CBX2, TXN, CYP4F11, CYP4F3, AKR1C1, AKR1B15, G6PD, PRDX1, TALDO1, EPT1, SRXN1, JAKMIP3, FTHL3, UCHL1, TXNRD1, Clorf131, CASKIN1, PGD, GPX2, OSGIN1, KIAA0319, CABYR, AIFM2, TRIM16, AKR1B10, GCLC, ABCC2, ETFB, IDH1, MAFG, NECAB2, ME1, PTGR1, PIR, GSR, RIT1, GCLM, ALDH3A1, NQO1, PKD1L2, NRG4, ABHD4, HRG, SLC7A11; and,
(d) tRCC signature group: FST, TRIM63, SLC10A2, ANTXRL, ERW-2, SNX22, INHBE, SV2B, FAM124A, EPHA5, LUZP2, CPEB1, HOXB13, ALLC, KCNF1, NDRG4, GREB1, ASTN1, JSRP1, UBE2U, KCNQ4, MYO7B, BRINP2, C1QL2, CCDC136, SLC51B, CATSPERG, PMEL, BIRC7, PLK5, ADARB2, CFAP61, TUBB4A, PLIN4, ABCB5, SYT3, HCN4, CTSK, SPACA1, TRIM67, NMRK2, LGI3, ARHGEF4, NTSR2, KEL, SNCB, PLD5, ADGRB1, CYP17A1, IGFBPL1, TRIM71, SLC45A2, TP73, IP6K3, HABP2, RGS20, IGFN1, CDH17.

18-23. (canceled)

24. The method of claim 1, wherein generating the RC TME signature further comprises normalizing the gene group scores, wherein the normalizing comprises applying median scaling to the gene group scores.

25-30. (canceled)

31. The method of claim 1, wherein the plurality of RC TME types comprises: RC TME type A, RC TME type B, RC TME type C, RC TME type D, and RC TME type E.

32. The method of claim 1, further comprising:
identifying at least one therapeutic agent for administration to the subject using the RC TME type of the subject.

33-39. (canceled)

40. A method for determining a renal cancer (RC) myogenesis signature for a subject having, suspected of having, or at risk of having renal cancer, the method comprising:
using at least one computer hardware processor to perform:
(a) obtaining RNA expression data for the subject, the RNA expression data indicating RNA expression levels for at least some of the genes in the gene group listed in Table 2; and
(b) generating a myogenesis signature for the subject using the RNA expression data, the myogenesis signature consisting of a gene group score for the gene group listed in Table 2,
the gene group score determined using the RNA expression levels.

41-47. (canceled)

48. The method of claim 40, wherein the RNA expression levels comprise RNA expression levels for at least three of the following genes: CASQ1, TNNI1, MB, MYLPF, MYH7, CKM, MYL2, MYL1, CSRP3, ACTA1, MYOZ1, TNNT3, TNNC2, and TNNC1.

49-55. (canceled)

56. A method for predicting the likelihood of a subject responding to an immuno-oncology (IO) agent, the subject having, suspected of having, or at risk of having renal cancer, the method comprising:
using at least one computer hardware processor to perform:
(a) generating, using RNA expression data that has been obtained from a subject, a set of input features, the set of input features comprising at least two of the following features:
(i) an RC TME type for the subject;
(ii) RNA expression levels for one or more of the following genes: PD1, PD-L1, and PD-L2;
(iii) an ECM associated signature for the subject;
(iv) an Angiogenesis signature for the subject;
(v) a Proliferation rate signature for the subject; and
(vi) a similarity score indicative of a similarity of an RC TME signature for the subject to RC TME signatures associated with RC TME type B and/or RC TME Type C samples;
(b) providing the set of input features as input to a machine learning model to obtain a corresponding output indicating a responder score, the responder score indicative of a likelihood that the subject responds to the immuno-oncology (IO) agent;
(c) identifying the subject as likely to have an increased likelihood of responding to the IO agent when the responder score is greater than a specified threshold.

57. The method of claim 56, wherein generating the set of input features comprises:
determining the RC TME type for the subject.

58. (canceled)

59. The method of claim 56, wherein generating the set of input features comprises:
determining the ECM associated signature for the subject using the RNA expression data by performing ssGSEA on the RNA expression data for at least three of the “ECM associated signature” genes listed in Table 1.

60-67. (canceled)

68. The method of claim 56, wherein generating the set of input features comprises:
determining the similarity score by comparing the gene group scores of the RC TME signature of the subject to an average of gene group scores of a plurality of RC TME signatures from RC TME type B samples and/or an average of gene group scores of a plurality of RC TME signatures from RC TME type C samples.

69. (canceled)

70. The method of claim 56, further comprising identifying the subject as being:
(i) “IO-low” when the responder score is ≤0.05;
(ii) “IO-medium” when the responder score is ≥0.05 and <0.5; or
(iii) “IO-high” when the responder score is ≥0.5.

71. (canceled)

72. The method of claim 56, wherein the specified threshold is 0.5, and the method further comprises identifying an IO agent for administration to the subject when the responder score of the subject is above the specified threshold.

73-75. (canceled)

76. The method of claim 56, further comprising determining whether the subject comprises one or more of the following biomarkers prior to performing step (b):
(i) Ploidy >4;
(ii) a value of a RC myogenesis signature for the subject is greater than 4;
(iii) one or more mTOR activating mutations; and/or
(iv) one or more mutations in a gene or genes associated with antigen presentation.

77. The method of claim 76 further comprising identifying the subject as having a responder score of 0 when the subject comprises one or more of the biomarkers.

78. A method for predicting the likelihood of a subject responding to tyrosine kinase inhibitor (TKI), the subject having, suspected of having, or at risk of having renal cancer, the method comprising:
using at least one computer hardware processor to perform:
(d) generating, using RNA expression data that has been obtained from a subject, a set of input features, the set of input features comprising at least two of the following features:
(vii) a Macrophage signature for the subject;
(viii) an Angiogenesis signature for the subject;
(ix) a Proliferation rate signature for the subject; and
(x) a similarity score indicative of a similarity of an RC TME signature for the subject to RC TME signatures associated with RC TME type B samples;
(e) providing the set of input features as input to a machine learning model to obtain a corresponding output indicating a responder score, the responder score indicative of a likelihood that the subject responds to the TKI;
(f) identifying the subject as likely to have an increased likelihood of responding to the TKI when the responder score is greater than a specified threshold.

79. The method of claim 78, wherein generating the set of input features comprises:
determining the RC TME type for the subject.

80-88. (canceled)

89. The method of claim 78, wherein generating the set of input features comprises:
determining the similarity score by comparing the gene group scores of the RC TME signature of the subject to an average of gene group scores of a plurality of RC TME signatures from RC TME type B samples.

90. (canceled)

91. The method of claim 78, further comprising identifying the subject as being:
(i) “TKI-low” when the responder score is ≤0.75;
(ii) “TKI-medium” when the responder score is ≥0.75 and <0.95; or
(iii) “TKI-high” when the responder score is ≥0.95.

92. The method of claim 78, wherein the specified threshold is 0.95.

93. The method of claim 78, further comprising identifying a TKI for administration to the subject when the responder score of the subject is above the specified threshold or wherein the subject is identified as being “TKI-medium” or “TKI-high”.

94-110. (canceled)
